SEQUENCE LISTING (19 sequences)

SEQ ID NO: 1 - DNA, 25 bp, Mustela putorius furo
tgatgattatgggagagttggagcc

SEQ ID NO: 2 - DNA, 22 bp, Mustela putorius furo
gcatgctttgatgacactcctg

SEQ ID NO: 3 - DNA, 30 bp, Artificial Sequence
  Primer
ccatcgatggcacccctgtgttatctttct

SEQ ID NO: 4 - DNA, 35 bp, Artificial Sequence
  Primer
ccggtacctatcagatccaggaaaactgagagcag

SEQ ID NO: 5 - DNA, 44 bp, Artificial Sequence
  Primer
ccatcgatgcggccgcgagctcgcctggcaccatcaaagaaaac

SEQ ID NO: 6 - DNA, 36 bp, Artificial Sequence
  Primer
ggactagtggatccgatggcctttcctttggatgga

SEQ ID NO: 7 - DNA, 21 bp, Artificial Sequence
  Primer
tggtttcaagggaatggggtc

SEQ ID NO: 8 - DNA, 22 bp, Artificial Sequence
  Primer
aagcgaaggagcaaagctgcta

SEQ ID NO: 9 - DNA, 25 bp, Artificial Sequence
  Primer
ggtgcaggaggtgttttgtcataga

SEQ ID NO: 10 - DNA, 22 bp, Artificial Sequence
  Primer
gctaaagcgcatgctccagact

SEQ ID NO: 11 - DNA, 21 bp, Artificial Sequence
  Primer
cggaccgctatcaggacatag

SEQ ID NO: 12 - DNA, 20 bp, Artificial Sequence
  Primer
tacgaaatgcagcaagcgcc

SEQ ID NO: 13 - DNA, 21 bp, Artificial Sequence
  Primer
aggtgtcattctattctgggg

SEQ ID NO: 14 - DNA, 19 bp, Artificial Sequence
  Primer
cccaggcatccctgaaact

SEQ ID NO: 15 - DNA, 25 bp, Artificial Sequence
  Primer
tgatgattatgggagagttggagcc

SEQ ID NO: 16 - DNA, 22 bp, Artificial Sequence
  Primer
ttgatggtgccaggcatgatcc

SEQ ID NO: 17 - DNA, 21 bp, Artificial Sequence
  Primer
tggcggccgttactagtggat

SEQ ID NO: 18 - DNA, 123 bp, Artificial Sequence
  cDNA
agtactcagagcctcgagtacccttacgacgttcctgattacgctgctagctacccttacgacgttcctgattacgcttacccttacgacgttcctgattacgctgcttctataaatagcagt

SEQ ID NO: 19 - protein, 41 aa, Artificial Sequence
  A synthetic oligopeptide
STQSLEYPYDVPDYAASYPYDVPDYAYPYDVPDYAASINSS

(19) United States
(12) Patent Application Publication
(10) Pub. No.: US 2011/0054247 A1
(43) Pub. Date: Mar. 3, 2011

(54) METHODS FOR CLONING FERRETS AND TRANSGENIC FERRET MODELS FOR DISEASES

(75) Inventors: XINGSHEN SUN, CORALVILLE, IA (US); YALING YI, CORALVILLE, IA (US); GREGORY H. LENO, CORALVILLE, IA (US); JOHN F. ENGELHARDT, IOWA CITY, IA (US); ZIYING YAN, IOWA CITY, IA (US); MICHAEL J. WELSH, RIVERSIDE, IA (US); CHRIS ROGERS, NORTH LIBERTY, IA (US); Xiaoming Liu, Iowa City, IA (US)

(73) Assignee: UNIVERSITY OF IOWA RESEARCH FOUNDATION, IOWA CITY, IA (US)

(21) Appl. No.: 12/835,102

(22) Filed: Jul. 13, 2010

Related U.S. Application Data
(63) Continuation-in-part of application No. 12/397,583, filed on Mar. 4, 2009.

(51) Int. Cl. A61D 19/04; A01K 67/00; C12N 5/10; A01N 1/02

(52) U.S. Cl. 600/34; 800/9; 435/325; 435/1.3

(57) ABSTRACT

The invention provides a transgenic Mustelidae in which a gene associated with a human disease or condition comprises a targeted genetic modification, and uses thereof. Also provided is a method to cryopreserve Mustelidae embryos or cells, and to enhance the number of live offspring from cryopreserved Mustelidae embryos.

### RELATED APPLICATION

This application is continuation in part of U.S. application Ser. No. 12/397,583, filed on Mar. 4, 2009, which claims priority under 35 U.S.C. §119(e) from U.S. Provisional Application Ser. No. 60/033,700, filed Mar. 4, 2008, the disclosures of which are incorporated herein by reference in their entirety.

### STATEMENT OF GOVERNMENT RIGHTS

The invention was made with a grant from the Government of the United States of America (grants No. HL61234 and HL 099516 from The National Institutes of Health). The Government has certain rights in the invention.

### BACKGROUND

Many human diseases and conditions are caused by gene mutations. Substantial effort has been directed towards the creation of transgenic animal models of such diseases and conditions, to facilitate the testing of approaches to treatment, as well as to gain a better understanding of disease pathology. Early transgenic animal technology focused on the use of embryonic stem cells and pronuclear injection in the mouse, while more recent efforts, which have been bolstered by the development of somatic cell nuclear transfer (SCNT), have included larger animals, including pigs, cows, and goats. Pronuclear injection results in random not targeted integration in contrast to gene targeting methods which are employed with SCNT. SCNT and gene targeting has resulted in the production of, for example, pigs in which the gene encoding α-1,3-galactosyltransferase has been knocked out, which may generate organs that can be used in xenotransplantation (see, e.g., Lai et al., 2002). Additional applications of this technology include the production of large quantities of human proteins (e.g., therapeutic antibodies; see, e.g., Grosse-Hovest et al., 2004). Substantial benefits may be obtained by the use of somatic cell nuclear transfer technology in the production of large animal models of human disease.

An example of a disease caused by gene mutations is cystic fibrosis (CF), which is an inherited disease that affects many organs of the body, including the lungs, pancreas, sweat glands, liver, and organs of the reproductive tract. The disease is characterized by abnormalities in fluid secretion, which can lead to diverse physiological problems. For example, in the lungs of CF patients, secreted mucus is unusually heavy and sticky, and thus tends to clog small air passages, making it difficult for patients to breath and leading to bacterial infection and inflammation. Repeated lung infections and blockages in CF patients can cause severe, permanent lung damage. Other features of CF arise from the clogging of ducts leading from the pancreas to the small intestine, which blocks the transport of critical digestive enzymes such as amylase, protease, and lipase. This can lead to problems including incomplete digestion, diarrhea, bowel blockage, and weight loss. Digestive complications of CF can also be caused by blockage of liver bile ducts. Due to these and other features of the disease, CF causes progressive disability in patients and ultimately leads to early death.

CF is caused by the presence of a mutation in the gene encoding the cystic fibrosis transmembrane conductance regulator (CFTR) protein, which is a chloride channel found in the membranes of epithelial cells lining passageways of the lungs, liver, pancreas, digestive tract, and in the skin. The disease is autosomal recessive, and thus CF patients have mutations in both CFTR alleles, while asymptomatic CF carriers have mutations in only one allele. There are more than 1,200 different known mutations of the CFTR gene that can lead to cystic fibrosis in humans, with some mutations causing milder symptoms than others. However, about 70% of people with CF have the disease due to a particular gene mutation, a deletion of three nucleotides, leading to the loss of a phenylalanine that is normally present at position 508 of the CFTR protein. The loss of phenylalanine at this position results in improper CFTR protein folding, which targets it for degradation before it even reaches the cell membrane. This form of the disease, often referred to as ΔF508, is thus both the most common and the most severe form of the disease.

There is no cure for CF. Current approaches to treatment include the use of mucous thinning drugs, digestive enzyme supplementation, bronchodilators, respiratory therapy, antibiotics, and lung transplantation. Even given the availability of these approaches to treatment, as the disease progresses, patients typically suffer from an increasingly poor quality of life. New approaches to treating diseases such as CF, which may be identified, for example, by the use of large animal models, are therefore needed for this and other devastating diseases.

### SUMMARY OF THE INVENTION

Genetically-defined nonhuman animal species for biomedical research are the foundation for several multibillion dollar businesses that specialize in mice, rats, and rabbits. However, those species may not be useful as models for a particular disorder because they do not display the human disease phenotype. Ferrets may be a superior species for modeling many human diseases as they share numerous cellular and physiological features with humans that mice, rats and rabbits fail to adequately replicate. Further, ferrets are the only small carnivore in which nuclear transfer has been used to produce cloned animals with specific genetic modifications, allowing for the modeling of human genetic disorders. Also, ferrets have a reproductive time equivalent to rabbits and have similar costs for housing, and are the model of choice for studying pandemic viruses such as avian flu, swine flu and SARs.

The present invention provides methods for cloning ferrets and other members of the Mustelidae family from cells such as fibroblasts, including the use of senescent fibroblasts as donor cells for nuclear transfer. Cloning from senescent fibroblasts has been difficult for multiple species and has hindered progress in the generation of genetically defined cloned species. That is because primary somatic cells have a relatively short lifespan and extended cell culture is needed for selection of properly targeted cell colonies. Extended culture often leads to senescence before analyses is complete, which in turn results in a low number of cells for SCNT.

In one embodiment, the invention provides for the production of cloned domestic gene targeted ferrets such as those having a disrupted CFTR gene. The cloned CFTR gene disrupted ferrets were prepared using recombinant adeno-associated virus (rAAV) gene targeting in fetal ferret fibroblasts and a serial nuclear transfer cloning technique to rejuvenate senescent gene-targeted cells prior to cloning of full-term ferrets. The invention is not limited to the use fetal cells or fibroblasts, e.g., 19 day embryos have been cloned from adult cells (see FIG. 28), and cell types other than fibroblasts, e.g., endothelial cells such as lung cells, kidney cells, liver cells, or intestinal cells, or cells in the brain, may be employed. Using this serial cloning technique, healthy CFTR gene-disrupted male ferrets were obtained from recipient Jills, thus demonstrating the feasibility of AAV-mediated genetic manipulation in the ferret using somatic cell nuclear transfer (SCNT). Neonatal CF ferrets demonstrated many of the characteristics of human CF disease, including defective airway chloride transport and submucosal-gland fluid secretion, variably penetrant meconium ileus (MI), pancreatic, liver and vas deferens disease, and a predisposition to lung infection in the early post-natal period. Severe malabsorption by the gastrointestinal tract was the primary cause of death in CF kits that escaped MI. Elevated liver function tests in CF kits were treatable by oral administration of ursodeoxycholic acid, and the addition of an oral proton-pump inhibitor improved weight gain and survival. To address the intestinal phenotype, four gut-corrected transgenic CFTR-knockout kits were cloned that expressed fCFTR specifically in the intestine. One clone passed feces normally and demonstrated no detectable fCFTR expression in the lung or liver. Animal modeling of CF has been challenging, with species- and strain-specific differences in organ biology and CFTR function influencing the emergence of disease pathology. Thus, the CF ferret models described herein may be useful tools for dissecting disease pathogenesis and developing treatments. Moreover, the SCNT approach may also be of utility in genetic manipulation and disease modeling for other genes and in other species.

The cloning methods of the invention may be employed with transgenic of non-transgenic cells. IFor example, cloning method is for animals with specific phenotypes, such as CF knockout animals without meconium ileus, with or without CF related diabetes, or CF animals with severe or mild lung disease. Propagation of these phenotypes by SCNT can produce larger cohorts to study treatments of genetically identical animals. Additionally, breeding of cloned animals to other clones can quickly allow for the identification of modifier genes for the unique phenotypes based on small nucleotide polymorphism maps of the genome. In one embodiment, animals that are resistant to certain pathogens, e.g., SARS or H1N1/H5N1 infection, or highly susceptible to transmission of pathogen infection, are cloned, e.g., making drug screening easier. Such animals could also be used to genetically track modifier genes that control infection by these pandemic viruses. Basically, cloning animals with specific genetic-based sub-phenotypes can be very helpful in evaluating the pathophysiology of a disease, developing drugs, and also for tracking modifier genes.

The invention thus provides a transgenic Mustelidae model of a human disease or condition, in which a gene associated with the disease or condition comprises a targeted genetic modification, e.g., a targeted insertion, and optionally including a transgene for expression of a gene of interest, e.g., one directly or indirectly (e.g., one associated with the severity or penetrance of the disease or condition) associated with the disease or condition. In one embodiment, the transgene has a cDNA encoding a gene product of interest. In one embodiment, the transgene has genomic sequences encoding a gene product of interest. In one embodiment, the gene of interest is expressed from a tissue-specific transcription regulatory element, e.g., a tissue-specific promoter. In one embodiment, the gene of interest is expressed from a constitutive promoter. In one embodiment, a targeted genetic modification results in gene disruption (a gene “knock-out”). In one embodiment, the modification is an insertion of a nucleic acid molecule having a disease-associated gene, such as a mutant gene, e.g., one encoding a variant protein. In one embodiment, the modification is an insertion of a heterologous nucleic acid molecule, e.g., a selectable marker gene or one encoding an antibody heavy or light chain or a single chain Fv. In one embodiment, the modification is insertion of a heterologous nucleic acid molecule that allows for indexing of gene expression from an endogenous promoter, e.g., insertion of a reporter gene. In one embodiment, the insertion of a selectable marker gene or reporter gene results in a transcriptional reporter (e.g., insertion of genes encoding antibiotic resistance, chloramphenicol acetyltransferase, fluorescent proteins, such as GFP, or enzymes, e.g., luciferase, 3-galactosidase and the like) which produces a chimeric RNA transcript with an in-frame fusion of the selectable marker or reporter coding region. In one embodiment, the modification includes a coding region of the gene with a stop codon or frame shift, resulting in a RNA transcript with a truncated coding region. In one embodiment, the modification is insertion of a vector for overexpression of a wild-type gene, e.g., under the regulation of a cell-specific endogenous promoter. The transgenic nonhuman animal may be heterozygous or homozygous for the modification. Moreover, heterozygous cloned animals may be cross bred, e.g., F1 siblings (clone×clone matings), yielding outbred but genetically similar progeny, which may be useful in modifier genetic studies (see FIG. 8). Heterozygotes may also be bred to cloned “knock out” transgenics, e.g., those that are a genetic disease model, yielding progeny which may also be useful in modifier genetic studies (see FIG. 8). For example, if both male and female fibroblasts are gene targeted for the same disease mutation, multiple genetically identical male and female cloned animals can be bred to give colonies of genetically similar (e.g., genetic siblings) offspring with disease. In addition, cloned genetic disease nonhuman animals useful in modifier gene research may be obtained from a nonhuman animal BAC genomic library generated from a single female nonhuman animal fetus and fetal fibroblasts that were grown from this fetus and then cryopreserved. These cells can be expanded indefinitely by SCNT cloning of new fetuses using methods described herein. Gene targeting in these cells to create a disease model, followed by somatic cell nuclear cloning, gives rise to animals with a single genetic modification on the background of a known genome sequence. This allows for the identification of modifier genes of disease through the breeding of cloned ferrets. In one embodiment, both alleles of a gene may have an insertion, which can be identical to one another or can be different from one another.

Thus, the non-human animal models of the invention can include the modification(s) in one or both alleles of a gene in the genome of the transgenic animal, and the modification(s) can result in full or partial inactivation of the gene(s), e.g., via reduction or elimination of transcription or translation, or both, or production of a nonfunctional gene product, e.g., a truncated polypeptide, in cells that normally express the wild-type gene product. In one embodiment, the modification includes a heterologous (non-Mustelidae sequences) polynucleotide (e.g., a positive selection marker, such as an antibiotic resistance gene, a promoter, polyadenylation sequences or site-specific recombination sequences, or any combination thereof). In one embodiment, site-specific recombination sequences flank the positive selection marker, such as loxP sites, so that the positive selection marker may be removed by a recombinase (e.g., Cre recombinase).

In one embodiment, the transgenic Mustelidae of the invention is employed to identify an agent for inhibiting or treating at least one symptom of a human disease or condition. The method includes administering an agent to the transgenic Mustelidae and detecting or determining whether the administration results in an alteration of one or more symptoms of the disease or condition. In one embodiment, the disease or condition is cystic fibrosis.

The invention also provides isolated cells of the transgenic Mustelidae of the invention. The animal can be, for example, a ferret, badger, ermine or otter. In one example, the disease or condition is cystic fibrosis. In one embodiment, the gene includes one or more mutations in a cystic fibrosis transmembrane conductance regulator gene. The isolated cells of the invention can include the modification(s) in one or both alleles of the genes in the genomes of the cells, and the modification(s) can result in full or partial inactivation of the gene(s), or expression of an introduced mutant gene or overexpression of a wild-type gene. In one embodiment, the modification substantially eliminates expression of a functional gene product of the targeted gene in cells in which such expression normally takes place, absent the insertion. In one embodiment, the modification provides a dominant defect in the targeted gene and a gain of function that results in disease. In one embodiment, the cells are fetal cells, such as fetal fibroblasts, although other cell types are envisioned.

The invention further provides methods of making transgenic nonhuman animal models of diseases or conditions. The methods include providing a donor cell which is a fibroblast cell comprising an expression vector comprising a gene associated with a disease or condition or comprising a targeted disruption vector in a gene, which disruption results in a disease or condition. The nucleus of the donor cell or the donor cell is introduced into an enucleated oocyte to generate an embryo and the reconstructed embryo is transferred into a surrogate female. In one embodiment, the donor cell is a fetal fibroblast. In one embodiment, the donor cell is introduced to an enucleated oocyte to generate the embryo. In one embodiment, the donor cell includes one or more modifications in one allele of a gene, and the method is carried out to introduce one or more modifications into the other allele of the same gene. In one embodiment, the donor cell includes one or more modifications in one allele of a gene, and the method is carried out to introduce one or more modifications in one or both alleles of a different gene. The methods further involve breeding an animal that is born from the surrogate female to obtain a homozygous mutant for one or more genes.

In one embodiment, the invention provides a method of making a transgenic Mustelidae model of a disease or a condition, e.g., cystic fibrosis. In one embodiment, the Mustelidae is a ferret. In one embodiment, the donor cell employed in the method is a fetal fibroblast. In one embodiment, the donor cell is from a transgenic Mustelidae, e.g., one having a knock out in the CFTR gene. In one embodiment, to generate an embryo for use in the method, a donor cell and oocyte are subjected to an electrical pulse. In one embodiment, the targeted disruption in a donor cell results in a donor cell that does not express the gene. In one embodiment, the donor cell has an expression vector that encodes a variant protein associated with the disease or condition. In one embodiment, the overexpression of the gene in the expression vector in the donor cell is associated with the disease or condition. In one embodiment, the gene in the donor cell is a cystic fibrosis transmembrane conductance regulator gene, e.g., a human cystic fibrosis transmembrane conductance regulator gene. In one embodiment, the donor cell has a knock out in the CFTR gene and an expression vector with a transgene that encodes CFTR, e.g., a wild-type CFTR or a variant CFTR with substantially the same activity as a wild-type CFTR. For example, the variant CFTR may include sequences useful for detecting or isolating the variant CFTR, for instance, His tag sequences. In one embodiment, the expression vector includes a tissue- or cell-specific promoter operably linked to the open reading frame for CFTR. In one embodiment, the vector is introduced as an isolated linearized nucleic acid molecule. In one embodiment, the vector is introduced using rAAV. The method may further include obtaining transgenic progeny from the surrogate female or breeding a transgenic progeny of the surrogate female to obtain a progeny that is homozygous for the vector or the targeted disruption.

The invention also includes methods of identifying agents that can be used in the treatment of diseases or conditions (e.g., cystic fibrosis). These methods involve administering one or more agents to a transgenic animal, and monitoring the animal for one or more symptoms of the disease or condition. Detection of improvement in a symptom of the disease or condition indicates the identification of a compound that can be used in the treatment of the disease or condition.

The invention also provides highly efficient methods for archiving embryos such as ferret embryos from stocks that employs a method of cryopreservation (vitrification) using a pipette chamber technique (PCT). As described hereinbelow, ferret embryos at the morula (M), compact morula (CM) and early blastocyst (EB) stages were vitrified using an Eppendorf microloader pipette tip as the housing vessel. The rate of in vitro development was significantly higher among embryos vitrified at the CM and EB stages, relative to those vitrified at the morula stages. No developmental differences were observed when comparing CM and EB vitrified embryos with non-vitrified control embryos. In addition, no differences in the actin cytoskeleton were noted between control embryos and embryos vitrified at any developmental stage. Thirty-four thawed vitrified embryos were cultured for shorter periods in vitro and subsequently transferred into five recipient females. All five recipients became pregnant and 74% of transferred embryos gave rise to live births at 42 days. However, vitrified embryos cultured in vitro for longer periods before ET did not produce live pups. These data suggest that the pipette-tip vitrification technique described herein, that reduces vessel size and loading volume, and the identification of the optimal stage of Mustelidae embryo vitrification with respect to the degree of survival and the developmental potential following vitrification, thawing and embryo transfer, improves the developmental efficiency of thawed ferret embryos following ET and may also be the most efficient method for the preservation and conservation of other mustelid species.

The invention also provides a method to cryopreserve embryos of nonhuman carnivores. The method includes providing one or more morula embryos, compact morulas or early blastocyst embryos in cryopreservation medium, introduce one or more of the embryos to a vessel with an inner diameter of about 0.2 mm to about 0.3 mm and a wall thickness of about 0.01 mm to about 0.05 mm, and subjecting the vessel with the one or more embryos to cryopreservation. The method does not include removal of cytoplasmic lipids prior to cryopreservation. The embryos may be from transgenic or nontransgenic animals. Embyros cryopreserved by the method of the invention may be thawed and transferred to pseudopregnant females, e.g., within less than 48 hours of thawing. As described hereinbelow, the use of the method resulted in an increased number of live birth ferrets from cryopreserved ferret embryos. Thus, the invention provides one or more cryopreserved embryos prepared by the method.

Also provided is a method to prepare pseudoembyros for cryopreservation.

### BRIEF DESCRIPTION OF THE FIGURES

FIG. 1. CFTR gene targeting in ferret fibroblasts. (A) Genomic fragment containing exon-10 of the ferret CFTR gene (top) and the rAAV CFTR targeting vector (bottom). Arrows mark nested primers used for PCR screening of targeting events. Restriction sites and probes used for Southern blot confirmation of targeting events are also shown. Diagram is not to scale. (B) Southern blot analysis of fibroblasts derived from the CL-B96 CFTR gene targeted 21 day nuclear transfer (NT) cloned ferret embryo using restriction sites and probes marked in panel A. The originating fibroblast line used for targeting is shown in lanes 1 and 4, while the CFTR gene targeted fibroblasts are shown in lanes 2 and 3. Arrowheads to the right of blots depict the non-targeted (solid) and gene-targeted (open) CFTR alleles.

FIG. 2. Cloning of CFTR targeted ferrets. (A) Cloned CFTR-targeted ferrets. Left top panel shows the first clone (sable coat color) at 5 weeks of age with its albino coat color non-cloned foster sibling. All other panels show sable clones with indicated ages. (B) Southern blot analysis of ear fibroblast DNA from the eight CFTR-targeted cloned ferrets (1-8) using AflII digested genomic DNA and the indicated CFTR and neomycin probes. NT=non-targeted unrelated ferret DNA. Arrowheads depict the non-targeted (solid) and gene-targeted (open) CFTR alleles. (C) Preweaning growth rate of CFTR-targeted ferret clones as compared to unrelated non-cloned ferrets. Results depict the mean (+/−SEM) for the indicated N in each group.

FIG. 3. The pipet chamber technique (PCT) for ferret embryo vitrification. (A) Eppendorf-tip used for embryo vitrification; (B) Schematic transverse section of the tip illustrating the inner diameter (ID) and the thickness of the outer wall; (C) Longitudinal view of tip with about 0.05 μL holding medium and two CM ferret embryos shown to illustrate the relative sizes of tip diameter and embryos.

FIG. 4. Ferret embryos before vitrification (pre-vitrification) and after thawing (post-vitrification thawing). Blastocysts obtained after in vitro culture of vitrified and thawed embryos (in vitro culture of thawed embryos). (A) Pre-vitrification morula embryos; (B) Thawed morula embryos; (C) Blastocyst derived from thawed morula embryo after 72 hours of in vitro culture; (D) Pre-vitrification compact morulae; (E) Thawed compact morula; (F) Blastocyst derived from thawed compact morula after 48 hours of in vitro culture; (G) Pre-vitrification early blastocysts; (H) Thawed early blastocysts; (J) Blastocyst derived from thawed early blastocyst after 48 hours of in vitro culture.

FIG. 5. Photomicrographs of vitrified, thawed, and in vitro cultured ferret blastocysts stained with Alexa Fluor 488-phalloidin (green) to label actin filaments. (A) Grade I blastocyst showing a precise, sharp restriction of actin staining to the cell borders in the embryo; (B) Grade II blastocyst showing a poorly-defined actin staining pattern within the embryo. Small clumps of actin staining were also observed within the cytoplasm.

FIG. 6. Classification of actin filament staining in vitrified and non-vitrified blastocysts. Proportion of Grade I (gray) and Grade II (black) blastocysts derived from vitrified (V) and non-vitrified control (C) morula (M), compact morula (CM), and early blastocyst (EB) ferret embryos following 24 hours of in vitro culture.

FIG. 7. Live ferret pups born from vitrified, thawed, and in vitro-cultured Sable embryos transferred to recipient albino Jills. (A, C) Five day-old pups with or without their foster mother; (B, D) Pups after 5 weeks with or without their foster mother.

FIG. 8. Schematic of the use of cloned ferrets for modifier genetics.

FIG. 9. Schematic of the use of cloned ferrets for environmental modifiers of disease using cloned ferrets.

FIG. 10. Summary of cloning methods for ferrets.

FIG. 11. Photomicrographs of A) gene targeted somatic cells, B) injection pipette with gene targeted somatic cells, C) injection of gene targeted somatic cells into an empty oocyte, and D) a pseudoembryo.

FIG. 12. Exemplary Mustelidae for cloning and embryo cyropreservation.

FIG. 13. A subset of CFTR^{-/-} kits are born with meconium ileus (MI). (A) The top panel is a schematic diagram of the targeted neomycin gene insertion into CFTR exon-10 that was used to generate the model, and indicates the locations of primers and probes used for genotyping. The second and third panels depict the results of PCR and Southern blot genotyping for a litter of six kits, with the genotypes indicated. The bottom panel depicts the detection of CFTR protein from the intestine by CFTR immunoprecipitation followed by in vitro phosphorylation in the presence of [γ-³²P]ATP and protein kinase A. (B) Four kits at birth, with genotype indicated. (C) Intestines from kits at 36 to 48 hours after birth, with genotype indicated, demonstrating the variability in occurrence of MI and microcolon. Arrows mark perforations in the intestine caused by MI. (D) Histological analysis of the distal ileum/colon in hematoxylin and eosin (H&E) or periodic acid-Schiff (PAS) stained sections, for a CFTR^{+/+} kit and CFTR^{-/-} kit suffering from MI. All kits with MI demonstrated similar histopathology with enhanced mucous production (i.e., enhanced PAS staining in purple). Those CFTR^{-/-} kits without MI demonstrated histology similar to that in the control animals (data not shown).

FIG. 14. Mean birth weight of ferret kits. Depicted is the distribution of birth weights from CFTR^{+/+}, CFTR^{+/-}, and CFTR^{-/-} kits. The number of kits in each group is below the genotype. The mean+/−SEM birth weight in grams is given in parentheses. There was no significant difference in birth weight among genotypes.

FIG. 15. Primary organ pathologies observed in newborn CFTR^{-/-} kits. (A) H&E-stained sections of the pancreas. Eosinophilic zymogen material filled exocrine acini (asterisks) of the CFTR^{-/-} pancreas. (B) H&E-stained sections of the liver and gall bladder (GB). Arrowheads mark the bile-filled canaliculi infrequently seen in all genotypes. (C) H&E-stained section of the lung demonstrating the type of lung lesions observed in CFTR^{-/-} newborn kits. The CFTR^{-/-} examples are from the same animal that passed stool in FIG. 1C and died at 48 hrs. Boxed regions are enlarged in the lower panels and demonstrate evidence of fibrin deposition, necrosis, bacteria, and/or inflammation. (D) Blood chemistries for alanine aminotransferase (ALT), total bilirubin (Tbil), and cholesterol (CHOL) in animals of the indicated genotypes (WT=CFTR^{+/-;+/-}; CF=CFTR^{-/-}). CF kits were divided into two groups with and without meconium ileus (MI). BL=below limits of detection. Blood was drawn at the time of euthanasia. Values depict the mean+/−SEM for N=5-9 animals in each group.

FIG. 16. Additional blood chemistry data on CF and WT kits. (A) Analysis of direct and indirect bilirubin levels in the plasma from WT (CFTR^{+/+;+/-}) and CF (CFTR^{-/-}) kits at 1-2 days old. The data demonstrate on average that indirect bilirubin predominates in the plasma of CFTR^{-/-} kits, however, both direct and indirect bilirubin levels were elevated in comparison to age-matched WT controls. Individual data points are given for each animal with the mean+/−SEM for each genotype. (B) Blood chemistries for alkaline phosphatase (AP), gamma glutamyl transferase (GGT), bile acids (BA), and blood urea nitrogen (BUN) in animals for the given genotypes (WT=CFTR^{+/-;+/-}; CF=CFTR^{-/-}). CF kits were divided into two groups with and without meconium ileus (MI). Blood was drawn at the time of euthanasia. Values depict the mean+/−SEM for N=5-9 animals in each group. No significant differences in ALP, BA, and BUN were seen between WT and CF kits. AP and GGT were selectively elevated in CFTR^{-/-} kits that suffered from MI.

FIG. 17. CFTR-knockout kits have a degenerate or absent vas deferens at birth. Panels depict H&E-stained sections of the vas deferens from two CFTR^{+/+} and two CFTR^{-/-} newborn kits. H&E-stained sections of both vasa deferentia from each animal are shown in the top two rows. The bottom row show enlarged photomicrographs for the boxed regions in the top panels. Arrows in the top two rows mark the vas deferens in wild type animals or the location of the absent or degenerate vas deferens in CF animals. The vas deferens of CFTR^{-/-} animal-1 was degenerate on side-1 and absent on side-2, while CFTR^{-/-} animal-2 demonstrated complete bilateral absence of the vas. Arrowheads mark remnant vas deferens epithelial cells in the enlarged image for CFTR^{-/-} animal-1. Micron bars are indicated for each row of photomicrographs.

FIG. 18. Segmental absence of the vas deferens in CFTR^{-/-} kits was observed to sometimes have altered adjacent segments of remnant vas deferens. H&E-stained sections of both vasa deferentia are shown from two CFTR^{-/-} kits (different animals then those shown in FIG. 17). Arrows mark serpentine (i.e. tortuous), degenerate, or absent vas deferens. Two different sections are shown for each vas deferens from CFTR^{-/-} animal-4 and demonstrate bilateral segmental absence of the vas deferens with serpentine morphologic changes in the remnant segment of vas deferens on each side. Ductule serpentine behavior has been associated with obstruction and/or duct proliferation in different tissues. Micron bars in each panel equal to 50 μm.

FIG. 19. Ursodeoxycholic acid (UDCA) treatment lowers plasma ALT levels and total bilirubin but does not enhance weight gain. Treatment with UDCA and omeprazole improves weight gain. (A) Weight gain of CFTR^{+/+;+/-} kits (N=4) and littermate CFTR^{-/-} kits that passed stool and did not have MI (N=4). One of the CFTR^{-/-} kits survived to 9.5 days but failed to gain weight after the time points shown. Data depict the mean+/−SEM. (B) H&E-stained sections of the kidney and perirenal adipose tissue (upper and middle panels) and lung from 5-9 day old kits. Adipocytes (middle row) demonstrate depletion of fat stores (fewer white spaces) in both CFTR^{-/-} kits. Three of the four CFTR^{-/-} kits treated with UDCA demonstrated lung lesions similar to that shown in the right panel of the bottom row. Scale bars are the same for all panels in each row. (C) Blood chemistries for UDCA-treated kits. (WT=CFTR^{+/-;+/-}; CF=CFTR^{-/-}). BL=below limits of detection. Blood was drawn at the time of euthanasia. Values represent the mean+/−SEM for N=3-4 animals in each group. (D) CFTR^{-/-} and CFTR^{+/+} kits treated with UDCA and omeprazole. Left panel demonstrates size at 45 days of age, and right panel depicts weight gain. The timing of oral pancreatic enzyme replacement is shown by the arrows, with the lipase units administered with each feeding marked above each arrow.

FIG. 20. Lung and liver histopathology observed in two 5 day old CFTR^{-/-} kits treated with UDCA. At birth, CFTR^{+/+} and CFTR^{-/-} littermates were gavaged with Golytely® to facilitate the rapid passage of meconium, and then orally with 150-200 μl Elecare containing UDCA (5 mg/kg) every 6 hours. Kits were also giving subcutaneous injection of metronidazole (5 mg/kg) four times per day, and were allowed to nurse normally. By 48 hours, the CFTR^{-/-} kits failed to gain weight in comparison to the CFTR^{+/+} control. The two CFTR^{-/-} kits represented in this figure were euthanized at 5 days due to respiratory distress and the CFTR^{+/+} control kit was also sacrificed at this time. (A) Lung histology for the indicated genotypes is shown. Histopathology in the two CFTR^{-/-} kits was similar, demonstrating multifocal bronchopneumonia with intralesional neutrophils, macrophages, hemorrhage, fibrin and bacteria that were sometimes observed in large clumps (see arrows). (B) Modified Gram stain of lung section presented in (A) demonstrated Gram negative bacterial colonies (see arrows). This modified Gram stain histochemically stains gram positive bacteria blue and gram negative bacteria red. The boxed region is enlarged in the inset and demonstrates inflammatory cells and surrounding Gram negative bacteria. (C) Liver histology for the indicated genotypes is shown. CFTR^{-/-} kits demonstrated a significant reduction in glycogen/lipid vacuolization (arrows), consistent with malabsorption and lack of weight gain by the animals. Inset photomicrograph in bottom row is enlarged and from the same sample. No evidence for cholestasis or focal biliary cirrhosis was noted in the CFTR^{-/-} kits. Extramedullary hematopoiesis (EMC) of varying degrees was seen but did not correlate with genotype. Micron bars for each row are marked in the first photomicrograph on the left.

FIG. 21. Airway defects observed in CFTR^{-/-} kits. (A, B) Transepithelial potential difference (TEPD) measurements in tracheal xenografts generated from CFTR^{+/+}, CFTR^{+/-}, and CFTR^{-/-} kits. Representative tracings of TEPD (A) following sequential addition of drugs to the lumen of the xenografts airway: 100 μM amiloride, Cr-free buffer, 10 μM forskolin/200 μM 8-ctp-cAMP, and 100 μM GlyH-101. The cumulative data for ΔVt responses to the various buffer changes is shown (B) for the indicated genotypes. Results depict the mean+/−SEM ΔVt responses for N measurements in 4 independent xenografts for each genotype (TEPD was evaluated 3-4 times for each xenograft on different days). (C, D) Analysis of submucosal gland secretion in tracheal xenografts from CFTR^{+/+}, CFTR^{+/-}, and CFTR^{-/-} kits. Representative en face photomicrographs (C) of glandular secretory droplets are shown (several marked by arrows): at baseline (unstimulated), in response to a 30 minute stimulation with 3 μM forskolin, and in response to a 30 minute stimulation with 1 μM carbachol. (D) Averaged data for glandular secretory rates in response to 3 μM forskolin or 1 μM carbachol for the indicated genotypes. Results depict the mean+/−SEM for N=10 CFTR^{+/+;+/-} xenografts and N=13 CFTR^{-/-} xenografts. †,*, mark comparisons that were significantly different using the student t-test (p<0.05). Typically 10-15 glands were measured for each xenograft sample. The average rate of secretion for all glands in a given sample was used to calculate the mean+/−SEM. The total number of glands analyzed was 110 for CFTR^{+/+;+/-} and 151 for CFTR^{-/-} xenografts.

FIG. 22. Generation of a gut-corrected transgenic CFTR^{-/-} ferret by somatic cell nuclear transfer. (A) Schematic diagram of the FAPBi-HA-fCFTR-PGK-Zeocin cassette used to generate transgenic ferrets expressing HA-tagged fCFTR under the direction of the FABPi promoter. (B) Primary fibroblasts were transfected with the linear transgenic fragment shown in (A), and selected pools were used for SCNT. Four cloned kits were born and the gross morphology of the intestine is shown. Clone-1 passed stool normally within 24 hours of birth, while clones-2, -3 and -4 suffered from MI and failed to pass stool. (C) PCR genotyping of the four transgenic FABP-HA-fCFTR/CFTR^{-/-} cloned kits. Genomic DNA from a CFTR^{+/+} kit served as a negative control, while plasmid DNA (pfCFTR) harboring the transgene cassette was used as a positive control. The PCR reactions were designed to specifically detect a segment of the HA-tag and fCFTR cDNA or the rat FABPi promoter as shown. (D) Detection of CFTR protein levels in intestinal lysates from the four FABP-HA-fCFTR/CFTR^{-/-} clones and a CFTR^{-/-} kit by CFTR immunoprecipitation, followed by in vitro phosphorylation in the presence of [γ-³²P]ATP and protein kinase A. (E) Comparison of CFTR protein levels using in vitro phosphorylation of immunoprecipitated CFTR from the intestine, lung and liver of FABP-HA-fCFTR/CFTR^{-/-} clone-1. Lanes show results for CFTR^{+/+}, CFTR^{-/-}, and FABP-HA-fCFTR/CFTR^{-/-} clone-1 kits. The fully glycosylated Band-C and partially processed Band-B forms of CFTR are shown (note that migration of transgenic fCFTR is slightly slower than that of endogenous CFTR, due to the presence of the HA-tag).

FIG. 23. Lung histopathology in CFTR^{-/-} kits treated with UDCA and omeprazole. (A-D) H&E stained sections of lungs from CFTR^{-/-} and CFTR^{+/+} kits reared on 150-200 μL Elecare containing UDCA (5 mg/kg) and omeprazole (5 mg/kg) every 6 hours. Kits were also giving subcutaneous injection of metronidazole (5 mg/kg) four times per day and were allowed to nurse normally. Panel A demonstrates lobar atelectasis in a CFTR^{-/-} kit (marked by an arrow). Panels C and D are enlargements of the boxed regions in panels A and B, respectively. Panel C demonstrates airway obstruction of the atelectatic lobe of the CFTR^{-/-} kit. Focal regions of atelectasis were also observed in other CFTR^{-/-} kits. (E-H) PAS stained section of the lung (E, F) and distal trachea (G, H) for each of the genotypes. In the CFTR^{-/-} kit shown, there was variably increased mucous change (enhanced PAS staining) in the surface airway epithelium and submucosal glands. This was not consistently observed in all CFTR^{-/-} kits. Micron bars for each row are marked in the left photomicrograph.

FIG. 24. Progressive pancreatic histopathology in CFTR^{-/-} kits treated with UDCA and omeprazole. H&E and Masson's trichome (MT) stained sections of pancreas from 19 day old CFTR^{-/-} and CFTR^{+/+} kits reared on 150-200 μL Elecare containing UDCA (5 mg/kg) and omeprazole (5 mg/kg) every 6 hours. Kits were also giving subcutaneous injection of metronidazole (5 mg/kg) four times per day and were allowed to nurse normally. The inset in the CFTR^{-/-} H&E panel is an enlargement of the region marked by an arrow and demonstrates inflammatory cells found in the dilated exocrine ducts/acini. Masson's trichome was used to detect fibrosis (marked by blue staining in interstitium, see arrows). Micron bars for each row are marked in the left photomicrograph.

FIG. 25. Bacteriology of bronchioalveolar lavage (BAL) fluid samples from CFTR^{-/-} kits treated with UDCA and omeprazole. CFTR^{-/-} and CFTR^{+/+} kits were reared on 150-200 μL Elecare containing UDCA (5 mg/kg) and omeprazole (5 mg/kg) every 6 hours. Kits were also giving subcutaneous injection of metronidazole (5 mg/kg) four times per day and were allowed to nurse normally. At the time of sacrificing animals a BAL was performed. Photographs of the aerobic plates are shown for four animals described in Table III. The labeling corresponds to animal numbers in Table 8 and the ranking of growth is given in brackets as defined in the Methods for Example 4 and Table 8 legend.

FIG. 26. Submucosal gland secretions in response to forskolin are reduced in CFTR^{-/-} xenografts. (A, B) Raw data set from a study comparing submucosal gland secretions between a CFTR^{+/+} (A) and CFTR^{-/-} (B). Large graph in each panel depicts glandular droplet volume for the indicated number of glands. Forskolin (3 μM) was added at 20 minutes. Inset graph depicts the mean+/−SEM for each dataset. Bottom panels are photomicrographs of glandular droplets taken during basal and stimulated measurements at the indicated times.

FIG. 27. Gavage tube used to feed newborn kits. (A) Gavage tubes were generated by modifying a flexible Eppendorf microloader tip. (B) The end of the gavage tube was fire polished as shown, to prevent perforation of the esophagus and stomach. (C) Items used to piece together the gavage tube included the polished microloader tip, a 16-gauge needle, and a 1 cc syringe. (D) The completed apparatus loaded with milk is shown.

FIG. 28. Improved cloning efficiency with rejuvenated cells from adult ferret fibroblast cells by somatic cell nuclear transfer (SCNT) (I) Skin fibroblasts are first collected from an adult ferret. (II) Cell are karyotyped as normal and then used for the first round of somatic cell nuclear transfer to rejuvenated the adult fibroblast cells. (III) 19 day cloned fetuses are collected and fibroblast generated. This image represents and embryo obtained from adult ferret fibroblasts demonstrating the feasibility of the SCNT. (IV) Karyotyped normal cells are used for a second round SCNT to produce cloned animals.

### DETAILED DESCRIPTION OF THE INVENTION

### Definitions

By “genetic modification,” “mutation,” or “disruption” of a gene is meant one or more alterations in gene sequences (including coding sequences and non-coding sequences, such as intron, promoter sequences, and 5′ and 3′-untranslated sequences) that alter the expression or activity of this gene by, for example, insertion (of, e.g., heterologous sequences, such as selectable markers and reporters, and/or termination signals), deletion, replacement, frame shift mutation, nonsense mutation, missense mutation, point mutation, or combinations thereof. In one example, the amino acid sequence encoded by the nucleic acid sequence has at least one amino acid altered as compared to a naturally-occurring sequence. For example, modifications of CFTR sequences are those that lead to one or more features of CF in transgenic animals including a disruption of both CFTR alleles. The modifications in the two CFTR alleles of such animals can be identical or different. Further, the modifications can result in a complete lack of functional CFTR production (as in the human 4F508 mutation), or can result in diminished functional CFTR production, as may be characteristic of less severe forms of the disease.

By “homozygous knock-out non-human mammal” is meant a mammal other than a human in which the two alleles of an endogenous gene (such as the CFTR gene) have been genetically targeted, resulting in a marked reduction or elimination of expression of a functional gene product, which is achieved by gene deletion or disruption. The genetic targeting event at both alleles may or may not be the same. Thus, a non-human mammal, in which the two alleles of an endogenous gene (such as a CFTR gene) have been genetically targeted by two different targeting vectors resulting in the null expression of the gene, would be considered as being a homozygous knock-out non-human mammal.

An example of a “knock-in mutation” is one resulting in the insertion of a mutation into an endogenous gene, for example, introducing the 4F508 or another CF mutation into a CFTR gene.

By “donor cell” is meant a cell from which a nucleus or chromatin material is derived, for use in nuclear transfer. Nuclear transfer can involve transfer of a nucleus or chromatin only, as isolated from a donor cell, or transfer of an entire donor cell including such a nucleus or chromatin material.

By “recipient cell” is meant a cell into which a donor cell, a donor cell nucleus, or donor cell chromatin is introduced. In one embodiment, recipient cells are enucleated prior to nuclear transfer. Examples of recipient cells include oocytes, fertilized zygotes, and two-cell embryos.

A “gene deletion” or “null” nonhuman animal means nonhuman animals having an insertion of a stop codon, an insertion resulting in a frame-shift, or a deletion which makes a target gene dysfunctional.

A “gene missense mutation” nonhuman animal means nonhuman animals having an insertion of a specific mutation found in human disease. The mutation can be dominant or recessive.

A “reporter gene” nonhuman animal has an insertion of a transcriptional reporter (fluorescent protein, luciferase, beta-galactosidase, and the like) downstream of a gene promoter to allow for indexing of gene expression.

### Methods and Transgenic Nonhuman Animals of the Invention

Since the birth of Dolly (Wilmut et al., 1997), the concept of combining SCNT with gene targeting in somatic cells has held tremendous potential for the development of new animal models of human disease such as CF. However, to date, this approach has failed to deliver on such potential due to technical challenges. A few laboratories have successfully applied SCNT with gene targeting technology in livestock for agricultural and biomedical (organ transplantation and protein production) applications (McCreath et al., 2000; Lai et al., 2002; Dai et al., 2002; Kuroiwa et al., 2004). However, no report has yet to apply this application to generate better non-rodent disease models. The development of robust gene targeting technologies that are compatible with SCNT have also been rate limiting, slowing progress in this area. Additionally, efficient nuclear transfer (NT) cloning procedures for potentially useful smaller species have lagged larger species such as sheep (8%-10%) (Wilmut et al., 1997; Schnieke et al., 1997), cattle (10-20%) (Wells et al., 2003; Yang et al., 2007), and pigs (5.5%) (Walker et al., 2002). These cloning efficiencies of 5-20% have been sufficient to generate gene-targeted sheep, cattle, and pigs (McCreath et al., 2000; Lai et al., 2002; Dai et al., 2002; Kuroiwa et al., 2004). While ferret cloning from highly reprogrammable somatic cumulous cells has been reported (Li et al., 2006), SCNT methods with fibroblasts (the best cell type for gene-targeting) have yet to be developed. Thus, further optimization of fibroblast-based SCNT cloning procedures are needed to facilitate the development of genetic models in smaller animals such as the ferret.

To improve oocyte quality for NT, a two step oocyte maturation process was employed, which improved oocyte quality and controlled oocyte age. The method described below employs ferret cells as an example but the methods may be employed with other nonhuman carnivores. In the first step, the oocytes were matured in vivo for 24 hours. For example, female sable coat-color ferrets (virgin, 6-7 months of age) and albino coat-color ferrets (primipara, 9-12 months of age) in estrus were used. Vasectomized male ferrets (Sable, 12 months of age) were used for mating to induce oocyte maturation in vivo in oocyte donor females. Immature oocytes were obtained from sable coat-color female ferrets mated with vasectomized male ferrets 24 hours prior to collection. To retrieve partly matured oocytes (mostly at MI stage), ferrets were euthanized by administration of pentobarbital sodium injection (50-100 mg/kg, i.p.). Only the preovulated follicles were punctured with fine forceps in DPBS (Gibco 14287) with 0.4% BSA to release the cumulus-oocyte complexes (COCs). Only oocytes with expanded cumulus were selected for in vitro maturation for 15 to 16 hours in medium-199 (Gibco 11150)+10% FBS+10 IU/mL eCG (equine chorionic gonadotrophin)+5 IU/mL of hCG (human chorionic gonadotrophin). This two step maturation protocol resulted in greatly improved oocyte developmental competence, concisely controlled oocyte age and avoided a fragmentation of matured oocytes once they were transferred in vivo.

Enucleation may be accomplished using a pizeo-driven micromanipulator system. For instance, using Nomarski optics, the first polar body and chromosome spindle were aspirated into the pipette with a minimal volume of oocyte cytoplasm with a 20 μm (inside diameter, ID) PeizoDrill glass pipette (Humagen™, Charlottesville, Va., USA). This improved the efficiency of enucleation by avoiding deformation of the oocyte during manipulation.

Somatic cells for donating nuclear genetic material may be from any source. Examples of cells that can be used as donor cells in making the transgenic animals of the invention may include fetal cells, placental cells, or adult cells. Specific examples of such cells for gene targeting include differentiated cells such as fibroblasts, epithelial cells, neural cells, epidermal cells, keratinocytes, hematopietic cells, melanocytes, chondrocytes, B-lymphocytes, T-lymphocytes, macrophages, monocytes, placental, and muscle cells. If a cell to be genetically altered is derived from an embryo or a fetus, the cell (e.g., a fetal cell or placental cell) can be isolated at any time during the gestation period until the birth of the animal, which may or may not be itself genetically altered.

For instance, while primary fibroblasts have a limited proliferative lifespan and usually rapidly senesce during expansion following transgenic manipulation which compromises the ability of these cells to be reprogrammed during the process of nuclear transfer, as described below, a procedure was developed to clone from a limited number of senescent cells. The cells were cultured in 96-well plate and around 100 cells were culture in each well. Prior to SCNT, the cells were trypsinized in a single well of a 96-well plate and then trypsin was neutralized by the addition of 20% FCS. Without centrifugation, the cells were directly transferred into the manipulation chamber for cell transfer. Due to the large size of senescent cells, a large, beveled pipette was used for transferring senescent cells into the perivitelline space (PVS) of enucleated oocytes. The diameter of the large, beveled pipette depends on size of the senescent cells. In one embodiment, the diameter was 35 to 40 μm. Normally for cloning, the same pipette is used for both enucleating of oocyte and inserting of somatic cell into PVS of enucleated oocytes.

To improve the fusion rate, the osmolarity of the fusion medium and manipulation may be altered. For instance, for ferret fibroblast enucleated oocyte fusion, the fusion medium contained 0.26 M mannitol, 0.1 mM MgCl₂, 0.1 mM CaCl₂, 0.5 mM Hepes, 0.01% (w/v) BSA. One somatic cell-oocyte couplet was transferred into fusion medium between a narrow gap (1 mm) of two electrodes. Then the couplet was aligned manually with a pipette under the microscope to make the contact surface between the oocyte and the donor cell parallel to the electrodes. Within 10 minutes, 15 to 20 couplets can be manipulated. Three hours after fusion, the fused embryos were then activated, e.g., with 5 mM ionomycin for 5 minutes in DPBS at room temperature, then with 2 mM 6-dimethylaminopurine for 3 hours in culture medium at 38.5° C. The activated oocytes were cultured, e.g., for 24 hours. The reconstructed embryos were transferred into pseudopregnant female and later, for instance, after 21 days gestation, the fetuses were collected and cells were established. Once the rejuvenating cell lines were established, these cells can be used for recloning. Efficiency of SCNT with the rejuvenated cell is similar to the efficiency of SCNT with normal fetal fibroblasts. This is different than in pigs for which SCNT cloning from cloned cells is very inefficient (i.e., sequential cloning).

Reconstructed embryos are introduced into surrogate females. For instance, primipara albino coat-color ferrets (9-12 months of age) in estrus were selected for the induction of pseudopregnancy using vasectomized males (albino, 12 months of age). For unknown reasons, this was important to establishing successful nuclear transfers into surrogate female ferrets. After 42 days gestation, the recipients were treated with prostaglandin (Lutalyse, 0.5 mg to 1 mg i.m.) to induce labor. If no kits were delivered within 3 hours, 0.3 mL of oxytocin was given to stimulate labor. If medical treatment was unsuccessful within 8 hours, a cesarean section was performed. If a recipient Jill failed to produce enough milk to feed the kits, the latter were fostered by another available Jill. This was very common in the cloning embryo transfer experiment. Since some Jills do not produce milk when fewer than five kits are born, procedures for cross-fostering cloned pups were developed to improve the survival rates.

The transgenic animals of the invention can be made using the following general strategy. Briefly, the genome of a cell (e.g., a fetal fibroblast) from an animal of interest, such as a ferret, is genetically modified by, for example, gene targeting by homologous recombination, to create a “donor cell.” Fetal fibroblasts are particularly useful for preparing NT reconstructed embryos such as those with defined mutations, which in turn are useful for preparing non-human animal models of disease. In one embodiment, the genetic modification results in at least partial inactivation of a gene associated with a particular disease or condition. The nucleus of such a genetically modified donor cell (or the entire donor cell, including the nucleus) is then transferred into a so-called “recipient cell,” such as an enucleated oocyte. After activation and, typically, a brief period of in vitro culture, the resulting embryo is implanted into a surrogate female in which development of the embryo proceeds. Typically, the donor cell, oocyte, and surrogate female are of the same species, but can be from different species.

Gene targeting carried out to make the cells and animals of the invention can result in gene inactivation by disruption, removal, modification, or replacement of target gene sequences. For example, inactivation can take place by the insertion of a heterologous sequence and/or a stop codon into a target gene. As is known in the art, inserted sequences can replace previously existing sequences in a gene or can be added to such sequences, depending on the design of the targeting construct. Also as is known in the art, the design of targeting constructs can be altered, depending upon whether it is desired to completely knock out the function of a gene or to maintain some level of reduced function. In the case of CFTR, for example, complete knock out of function is consistent with the most common form of CF (4F508), but other, less dramatic changes may be desirable for the generation of models of disease maintaining some CFTR function. Such changes may be achieved by, for example, replacement with sequences that are identical to wild-type sequences, except for the presence of specific mutations giving rise to features of the target disease. In other approaches, coding sequences are not altered or are minimally altered and, rather, sequences impacting expression of a target gene, such as promoter sequences, are targeted. In any case, selectable marker insertion is often desirable to facilitate identification of cells in which targeting has occurred. If desired, such markers or other inserted sequences can later be removed by, e.g., Cre-lox or similar systems.

Targeted gene modification requires the use of nucleic acid molecule constructs having regions of homology with a targeted gene (or flanking regions), such that integration of the construct into the genome alters expression of the gene, either by changing the sequence of the gene and/or the levels of expression of the gene. Thus, to alter a gene, a targeting construct is generally designed to contain three main regions: (i) a first region that is homologous to the locus to be targeted, (ii) a second region that is a heterologous polynucleotide sequence (e.g., encoding a selectable marker, such as an antibiotic resistance protein) that is to specifically replace a portion of the targeted locus or is inserted into the targeted locus, and (iii) a third region that, like the first region, is homologous to the targeted locus, but typically is not contiguous with the first region of the genome, Homologous recombination between the targeting construct and the targeted wild-type locus results in deletion of any locus sequences between the two regions of homology represented in the targeting vector and replacement of that sequence with, or insertion into that sequence of, a heterologous sequence that, for example, encodes a selectable marker. In the case of targeting transcriptionally inactive genes, such as, for example, the CFTR gene in fibroblasts, or a gene having only very low levels of transcription, the constructs of the invention can include a promoter, such as a PGK promoter, which drives expression of the selectable marker (e.g., Neo). Use of such promoters may not be required in cases in which transcriptionally active genes are targeted, if the design of the construct results in the marker being transcribed as directed by an endogenous promoter. Exemplary constructs and vectors for carrying out such targeted modification are described herein. However, other vectors that can be used in such approaches are known in the art and can readily be adapted for use in the invention.

In order to facilitate homologous recombination, the first and third regions of the targeting vectors (see above) include sequences that exhibit substantial identity to the genes to be targeted (or flanking regions). By “substantially identical” is meant having a sequence that is at least 80%, 90%, 95%, 98%, or 100% identical to that of another sequence. Sequence identity is typically measured using BLAST® (Basic Local Alignment Search Tool) or BLAST® 2 with the default parameters specified therein (see, Altschu et al., 1990); Tatiana et al., 1999)). These software programs match similar sequences by assigning degrees of homology to various substitutions, deletions, and other modifications. Thus, sequences having at least 80%, 90%, 98%, 99%, or even 100% sequence identity with the targeted gene loci can be used in the invention to facilitate homologous recombination.

The total size of the two regions of homology (i.e., the first and third regions noted above) can be, for example, approximately 2-25 kilobases (e.g., 4-20, 5-15, or 6-10 kilobases), and the size of the second region that replaces a portion of the targeted locus can be, for example, approximately 0.5-5 kilobases (e.g., 1-4 or 3-4 kilobases).

The targeting constructs can be included within any appropriate vectors, such as plasmid or viral vectors (e.g., adenovirus or adeno-associated virus vectors), which can be introduced into cells using standard methods including, for example, viral transduction, electroporation, or microinjection. One preferred example of the invention, which is described in detail in the experimental examples, below, employs an adeno-associated viral vector (AAV) (e.g., rAAV2, which can be made by standard methods from the wild-type AAV2 genome cloned in a plasmid, pAV2 (ATCC 37216)).

Other types of vectors, or more specifically other types of targeting construct delivery methods, are available. Cell transfection methods, including electroporation, nuclear injection, calcium phosphate and lipofection, can be used to deliver the targeting construct, though the disadvantages of inefficient transfection efficiency, cell toxicity, requirement of a pure (clean) targeting construct DNA sample, and poor ratio of homologous recombination to non-homologous recombination may outweigh the benefit of ease. If the gene is transcriptionally active in the cell type being used a promoterless selectable marker strategy may be employed so that antibiotic resistance will only be found in cell that have had a recombination event within a transcribed unit.

The use of AAV to deliver the targeting construct offers many benefits. First, AAV1 (and other AAV serotypes) infects ferret fetal fibroblasts with efficiency. Second, AAV infection of ferret fetal fibroblasts results in little or no cell toxicity. Third, AAV infection results in the delivery of a single-stranded gene targeting construct directly to the nucleus. Single-stranded gene targeting vectors are thought to yield more efficient gene targeting and result in a more favorable homologous recombination to non-homologous recombination ratio (Hendrie and Russell, 2005).

Genetically targeted cells are typically identified using a selectable marker, such as neomycin. If a cell already contains a selectable marker, however, a new targeting construct containing a different selectable maker can be used. For example, as described hereinbelow, cells that were neomycin resistant were subsequently transfected with a marker conferring zeocin resistance. Alternatively, if the same selectable marker is employed, cells can be selected in the second targeting round by raising the drug concentration (for example, by doubling the drug concentration), as is known in the art. As is noted above, targeting constructs can include selectable markers flanked by sites facilitating excision of the marker sequences. In one example, constructs can include loxP sites to facilitate the efficient deletion of the marker using the Cre/lox system. Use of such systems is well known in the art, and a specific example of use of this system is provided below, in the experimental examples.

Upon obtaining cells in which a target gene has been targeted (one or both alleles, as described above), nuclear transfer can be carried out. Optionally, the genetically modified nuclear donor cells can be frozen prior to nuclear transfer. Recipient cells that can be used in the invention are typically oocytes, fertilized zygotes, or two-cell embryos, all of which may or may not have been enucleated. Typically, the donor and the recipient cells are derived from the same species. However, it is possible to obtain development from embryos reconstructed using donor and recipient cells from different species.

Recipient oocytes can be obtained using methods that are known in the art. As is known in the art, the donor nucleus or the donor cell itself can be injected into the recipient cell or injected into the perivitelline space, adjacent to the oocyte membrane. The nuclear transfer complex formed in this manner can be activated by standard methods, which may involve electrical fusion/activation or electrical fusion/chemical activation, as is described further below.

The invention provides animal models of human disease, which can be used in the identification and characterization of approaches for treating the diseases and conditions. In one embodiment, the animal models of the invention have been genetically modified to include one or more mutations in a gene associated with a particular disease or condition. The genetic modifications can result in the animals having one or more symptoms characteristic of the disease or condition. Animals exhibiting such symptoms are particularly advantageous in the development of therapeutic approaches, as candidate drugs and other approaches to treatment can be evaluated for effects on the symptoms in such animals, Thus, in addition to the animal models themselves, the invention also provides methods of using the animals for identifying and characterizing treatments. Further, the invention includes methods of making transgenic animal models and cells that can be used in these methods. The animal models systems, methods, and cells of the invention are described further, below.

The invention is described below in reference to animal models of CF, which are generated by mutation of the CFTR gene. However, the methods of the invention are also applicable to the development of animal models of additional diseases and conditions.

Cystic fibrosis (CF) is the most common autosomal recessive condition affecting Caucasians. Although the cystic fibrosis transmembrane conductance regulator (CFTR) gene—which encodes an epithelial chloride channel defective in CF—was cloned nearly two decades ago, progress toward treatments has been hindered by the lack of an animal model that reproduces the life-threatening lung infections observed in CF patients. While certain CF mouse models demonstrate phenotypic alterations in response to bacterial agarose bead challenge in the lung (Heeckeren et al., 1997), they fail to develop the natural progression of spontaneous disease seen in humans. This is most likely due to species-specific expression of alternative chloride channels in their airways (Grubb et al., 1994; Liu et al. 2004). The domestic ferret (Mustela putorius furo) has been considered as an alternative species for modeling CF, given its high level of conservation in lung biology with humans and known utility as a model for other types of lung infection such as SARS virus (Martina et al., 2003) and influenza virus (Subbaro et al., 2007).

The domestic ferret is an attractive alternative species to model CF for several reasons. First, ferrets and humans share a remarkably similar airway cytoarchitecture (Mercer et al., 1994; Leigh et al., 1986; Wang et al., 2001), a feature not shared between humans and mice. Second, the expression pattern of the CFTR gene is extremely similar in ferret and human airways (Sehgal et al., 1996 and Englehardt et al. 1992) with the highest levels in submucosal glands. Bioelectric and pharmacologic properties of the CFTR chloride channel in ferret airway epithelia is also similar to that seen in human airway epithelia (Liu et al., 2007). In contrast to mice, ferret and human tracheobronchial airways contain abundant submucosal glands that express high levels of CFTR (Sehgal et al., 1996 and Englehardt et al. 1992; Choi et al., 2000); these glands have been shown to be critical for airway innate immunity in the ferret (Dajani et al., 2005), as predicted for humans (Verkman et al., 2003; Wine et al., 2004). Gene transfer to ferret airway epithelia with several adeno-associated virus (AAV) serotypes has also been shown to be very closely conserved to that seen in human airway epithelia (Liu et al., 2007); again, a conservation not shared with mice (Liu et al., 2006). In addition, the ferret has a 42-day gestation time and reaches sexual maturity in 5 to 6 months (Fox et al., 1998), making it one of the more rapidly reproducing species for animal modeling by somatic cell nuclear transfer (SCNT). Together, these studies point to the potential of the ferret to be a good species on which to model CF lung disease in humans.

The invention includes animals in which only one allele of a targeted gene is disrupted, with the other allele remaining unaffected. These animals, which are referred to herein as “heterozygous” or “hemizygous” animals, can be used, for example, in breeding approaches to generate homozygous mutants, if desired, for example, in the case of diseases caused by homozygous recessive mutations. These animals can be used as animal models themselves, in the case of diseases caused by autosomal dominant mutations.

Also included in the invention are homozygous mutant animals, in which both alleles of a target gene are disrupted, by the same or different mutations. In addition to being obtainable by breeding approaches involving hemizygous animals, homozygous mutant animals can also be obtained using an approach in which a cell (e.g., a fetal fibroblast) including a mutation in one allele, such as a cell obtained from an animal produced using the method summarized above, is subjected to gene targeting by homologous recombination to achieve modification of the remaining allele of the same gene. The resulting donor cell can then be used as a source of a modified nucleus for nuclear transfer into a recipient cell, such as an enucleated oocyte, leading to the formation of a homozygous mutant embryo which, when implanted into a surrogate female, develops into a homozygous mutant animal.

The animal model also makes it possible to assess electrolyte transport by airway epithelia in vitro and in vivo, the volume of airway surface liquid in vitro and in vivo, the ion composition of airway surface liquid in vitro and in vivo, the airway surface liquid pH in the airway, and electrolyte transport in the small airways. It is also possible to measure respiratory mucociliary transport in vitro and in vivo. For assessing inflammation, several tests and assays can be carried out, including (but not limited to) assays of key markers of inflammation in amniotic fluid, fetal lung liquid, and bronchoalveolar lavage by using lung tissue histochemistry, large-scale gene expression profiling of pulmonary tissues, cytokine and cell assays, and proteomics. It is also possible to raise CF and non-CF animals such as ferrets in isolators under completely germ free conditions and to test for the development of pulmonary inflammation, and then selectively expose the ferrets to inflammatory stimuli including bacteria and viruses. In addition, the effect of the loss of CFTR function in airway epithelia on NFKB signaling, the function of secreted epithelial antimicrobials/host defense proteins, and in macrophages or neutrophils can be evaluated. The availability of the CF model allows tests of the early manifestations of CF disease, an important question that remains unanswered. The natural history of pulmonary infections in CF ferrets can also be monitored, leading to a determination of whether the airway epithelia of CF ferrets can be colonized by CF or ferret pathogens and/or non-pathogenic opportunistic organisms.

In one embodiment, the CFTR mutation in the transgenic nonhuman animals of the invention is one that results in defective protein production. Those mutations include nonsense mutations, frameshift mutations and splice site mutations, e.g., G542X, 3905 insT, and 621+G→T, respectively, that lead to protein instability and degradation. Those mutations may be in a nucleotide binding domain or a membrane spanning domain of CFTR. In one embodiment, the CFTR mutation in the transgenic nonhuman animals of the invention is one that results in defective protein processing. For instance, the protein may be missing or present in reduced amounts in the apical membrane, or mislocalized, e.g., CFTRΔF508 is mislocalized but if present in the cell membrane retains some or all of its function. Exemplary mutations that result in defective protein processing include ΔI507, ΔF508, S549I, S549R, A559T, or N1303K. In one embodiment, the CFTR mutation in the transgenic nonhuman animals of the invention is one that results in defective regulation. Because intracellular ATP regulates the opening of CFTR Cl⁻ channels through direct interactions with the nucleotide binding domains, mutations in nucleotide binding domains may result in a nucleotide binding domain with very little function, or when ATP is present a nucleotide binding domain that is less potent at stimulating activity. The resulting decrease in net Cl⁻ channel activity is likely responsible for the defective epithelial Cl⁻ permeability in patients bearing these mutations. Exemplary mutations that result in defective regulation include G551D, G551S, G1244E, S1255P, or G1349D. In one embodiment, the CFTR mutation in the transgenic nonhuman animals of the invention is one that results in defective conduction, e.g., the amount of current is reduced due to a reduction in the rate of ion flow through an open channel. Exemplary mutations that result in defective conduction include R117H, R334W or R357P. In one embodiment, the transgenic nonhuman animals of the invention have a combination of CFTR mutations, e.g., a mutation that results in defective processing and one that results in defective conduction.

The transgenic animals of the invention can be used in the identification and characterization of drug and other treatment methods for the disease or condition associated with mutation of the gene targeted according to the invention. In these methods, for example, a candidate therapeutic agent can be administered to an animal and the impact of the agent on a feature of the disease exhibited by the animal can be monitored. In one embodiment, the transgenic animals administered a test agent are monitored for an improvement in pancreatic activity, e.g., for serum trypsin-like immunoreactivity, fecal protease, fecal elastase, or serum isoamylase. Optionally, the methods can also involve exposure of the animals to environmental or other conditions known to contribute to or exacerbate the disease or condition. For example, in the case of CF animal models having impaired respiratory function, the effect of the drug on such function can be assessed by measurement of standard respiratory parameters. In another example, in the case of animals exhibiting impaired digestion, due to blockage of pancreatic and/or liver ducts, the effect of a treatment on digestion can be determined.

Although lung disease is the current main cause of mortality, patients suffer from CF disease in many other organs. Availability of a CF model allows new investigations and tests of therapeutics in the pancreas, intestine, sweat gland, liver, vas deferens, kidney, and other organs affected primarily or secondarily by CF. The screening methods of the invention can be carried out to test the efficacy of new compounds, combinations of new and old compounds, non-pharmaceutical treatments, and combinations of pharmaceutical and non-pharmaceutical treatments.

The invention has been described above in reference to mutation of the CFTR gene to generate non-human animal models of cystic fibrosis. As is stated above, the invention can also be used in the generation of transgenic, non-human animal models of other diseases and conditions associated with gene mutations. There are innumerable examples of such diseases and conditions known in the art, which can be included in this invention. Some specific examples include but are not limited to hypercholesterolemia and atherosclerosis (e.g., LDLR and APOE genes), cancer (e.g., p53, and BRCAI and BRCA2 genes), Huntington's disease (huntington gene), Duchene muscular dystrophy (dystrophin gene), amyotrophic lateral sclerosis (SOD1 and alsin genes), polycystic kidney disease (PKDI and PKD2 genes), sickle-cell disease (alpha/beta-globin), Hemophilia A (Factor VIII gene), Ataxia-telangiectasia (ATM gene), and Retinoblastoma (RB1 gene).

Possible mutations to these disease genes include knock-outs (by, e.g., insertion of a selection cassette), knock-ins (e.g., by point mutations that correspond to human disease mutations or a cDNA downstream of an endogenous promoter), and, in the case of Huntington's disease (and any other trinucleotide repeat expansion disorder family members), an expansion of the trinucleotide repeat to pathogenic sizes.

### Methods for Maintenance of Ferret Embryos

The development of an efficient cryopreservation technique for early embryos is critical for the long-term maintenance of valuable genetic traits and for the development of effective assisted reproductive technologies. Key examples include transgenic animals that model human diseases and endangered species (Tesson et al., 2005; Li et al., 2005). The domestic ferret (Mustela putorius furo) is a member of the Mustela family and has been used extensively as an animal model in biomedical research involving virology, reproductive physiology, and endocrinology. The ferret has marked similarities to humans in its airway structure and lung cell biology, and therefore, has the potential to become a model of choice for the study of genetic lung diseases including cystic fibrosis (CF) (Li et al., 2003). This species is also considered an excellent model for the recovery and conservation of related endangered species such as the black-footed ferret and the European mink (Li et al., 2006).

Embryo cryopreservation was first accomplished in the mouse (Whittingham et al., 1972) and since that time, a variety of methods have been used successfully with cattle, goats, and sheep. The development of cryopreservation methods for mustelid embryos, on the other hand, has lagged behind that of these other species. Initial efforts toward cryopreservation of Mustelidae began with the stoat (Mustela erminea) in 1993 (Ya et al., 1993), and it was a full ten years later before embryos from the domestic ferret were successfully cryopreserved (Lindeberg et al., 2003). In the latter case, embryos grown to the expanded blastocyst stage were preserved using a slow-rate freezing technique. In total, 93 frozen and thawed embryos were transferred into 9 recipient females, with 10% producing live pups (Lindeberg et al., 2003). The same group later reported a 16% live birth rate resulting from the use of embryos vitrified by an open pulled straw (OPS) method (Piltti et al., 2004). Thus, the OPS-based vitrification improved embryo development relative to slow rate freezing, yet the efficiency of the approach with respect to embryo transfer was still quite low.

These difficulties in the performance of OPS vitrification methods for the ferret may be due to the fact that embryos from carnivors are lipid droplet-rich relative to embryos from other species (Kizilova et al., 1998); the presence of large numbers of lipid droplets may make the ferret embryo more prone to damage during freezing (Dobrinsky et al., 2002; Leibo et al., 2002). This, coupled with the fact that the plastic straw used for vitrification in the OPS procedure is not reliably at the desired diameter (i.e., <0.8 mm), and also the fact that it floats in liquid nitrogen rather than remaining submerged, may contribute to the technique's lack of success in this species (Kong et al., 2000; Cho et al., 2002). Indeed, a larger, floating straw decreases the cooling rate during vitrification and may thus compromise embryo viability. An alternative approach to overcoming these problems involved the use of a glass micropipette (GMP) as a vessel for vitrification. The disadvantage of this method is that the GMPs are quite fragile, resulting in embryo loss (Cho et al., 2002). However, this change improved survival rates of vitrified bovine blastocysts, a finding that may be attributed to increasing the rate of freezing due to the vessel's small size and loading volume (Cho et al., 2002).

A significant problem when cloning from senescent gene-targeted ferret fibroblast is the limited numbers of cells to work with and the inability to cryopreserve the cells for later use in somatic cell nuclear transfer. After selection, senescent gene-targeted ferret fibroblast colonies often contain only several thousand cells. It is currently not feasible to cryopreserve such a small number of cells in a fashion that retains cell viability. Such a method, however, would be useful as it would allow for cells to be archived for later SCNT experiments. To this end, a method was developed to cryopreserve small numbers of gene-targeted cells in an empty oocyte. As described herein below, empty ferret oocytes were generated from oocytes that were not of sufficient quality for nuclear transfer experiments.

The invention will be further described by the following nonlimiting examples.

### Example 1

Somatic cell gene targeting combined with nuclear transfer cloning presents tremendous potential for the creation of new larger animal models of human diseases, as mouse disease models often fail to reproduce the human phenotype. This is the case in mice deficient for the cystic fibrosis transmembrane conductance regulator (CFTR), which have unsuccessfully modeled many aspects of human cystic fibrosis (CF) lung disease. As described below, a CFTR gene deficient model in the domestic ferret was produced using recombinant adeno-associated virus (AAV)-mediated gene targeting in fibroblasts followed by nuclear transfer cloning. The success of this approach included the development of a somatic cell rejuvenation protocol using serial nuclear transfer to produce live CFTR-deficient clones from senescent gene-targeted fibroblasts. From 472 reconstructed embryos transferred into 11 recipient Jills, 8 healthy male ferret clones heterozygous for an exon 10 disruption in the CFTR gene were obtained. This approach, which was used to generate the first genetically engineered ferrets, may be of significant utility in modeling not only cystic fibrosis but also other genetic diseases.

### Methods

Animals. Ferrets were purchased from Marshall Farms (North Rose, N.Y., USA). Female sable coat-color Jills (virgin, 6-7 months of age) and albino coat-color Jills (primipara, 9-12 months of age) were in estrus when delivered. Vasectomized male ferrets (albino, 12 months of age) were used for mating to induce follicular oocyte maturation in oocyte donor Jills and to induce pseudopregnancy in NT embryo surrogate Jill recipients. All ferrets were housed in separate cages under controlled temperature (20-22° C.) and long day light cycle (16 hours light: 8 hours dark). Ferret chow was obtained from Marshall Farms. The use of animals in this study was carried out according to a protocol approved by the University of Iowa Institutional Animal Care and Use Committee and conformed to or exceeded National Institutes of Health standards.

Collection of fetal ferret fibroblasts. Fetal ferret fibroblasts were obtained from 28 day post copulation (dpc) fetuses derived from a Sable (female)×Sable (male) mating (Marshall Farms) as described in Li et al. (2005). Each fetus was treated individually. Karyotype analysis was performed on each embryo line and only male fibroblasts with a normal chromosomal profile were used for SCNT and CFTR gene targeting with rAAV.

Cloning ferret CFTR genomic DNA. Ferret genomic DNA was extracted from internal organs collected from an E28 female fetus and used to construct a ferret genomic BAC library through the BACPAC Resource Center (www.bacpac.chori.orq) at Children's Hospital Oakland Research Institute. The average length of the ferret genomic DNA inserts in this library is about 150 kbp. BAC clones encompassing ferret CFTR exon 10 were isolated from this library after screening with the CFTR exon 10 probe, synthesized according to the partial ferret CFTR cDNA sequence (gb:S82688) (Sehgal et al., 1996). BAC DNA from the CFTR positive clone was prepared with the ΨCLONE BAC DNA isolation kit (Princeton Separations). Sequencing of the CFTR exon 10 and adjacent introns was initiated with two primers located inside the CFTR exon 10 region (primers: e10F: TGATGATTATGGGAGAGTTGGAGCC; SEQ ID NO:1, and e10R: GCATGCTTTGATGACACTCCTG; SEQ ID NO:2). Primer walking was used to sequence approximately 2 kb on each side of exon 10 to generate a contig for cloning of the targeting vector. Based on the obtained CFTR genomic sequence, primers for subcloning were designed and two 2.0 kb CFTR fragments containing exon 10 and flanking intronic sequence were retrieved from BAC DNA by PCR with AccuPrime Pfx supermix (Invitrogen). The PCR products were cloned into the pBlunt4PCR vector with Topo cloning kit (Invitrogen) and confirmed by sequencing. The resultant plasmids were designated as pTopo-Left and pTopo-right encompassing exon 10 and left arm or right arm introns, respectively.

Generation of CFTR-targeting proviral vector. To construct the AAV targeting vector centered on CFTR exon 10, a 0.97 kb left homologous arm and a 1.23 kb right homologous arm were retrieved by PCR from pTopo-Left and pTopo-Right, respectively. The primer set for the left arm was: 5′-ccatcgatGGCACCCCTGTGTTATCTTTCT-3′ (forward; SEQ ID NO:3) and 5′-ccggtacctatcaGATCCAGGAAAACTGAGAGCAG-3′ (reverse; SEQ ID NO:4). The right arm set was: 5′-ccatcgatgcggccgcgagctcGCCTGGCACCATCAAAGAAAAC-3′ (forward; SEQ ID NO:5) and 5′-ggactagtggatccGATGGCCTTTCCTTTGGATGGA-3′ (reverse; SEQ ID NO:6) (lower case letters indicate restriction enzyme sequences introduced for cloning). The reverse primer of the left arm and forward primer of the right arm are located at the center of exon 10. The two PCR products together with a 1.7 kb PGK promoter driven neomycin resistance expression cassette were assembled and finally cloned into a AAV2 proviral plasmid giving rise to vector harboring 2.3 kb ferret CFTR genomic DNA with a neomycin cassette inserted at the center of exon 10. The rAAV-2 targeting virus (AV.CFtarg) was produced using a triple plasmid transfection procedure in 293 cells, and was purified over an iodixanol cushion followed by ion exchange HPLC (Yan et al., 2006).

Screening for CFTR-targeted fibroblast clones. Targeting was initiated by infecting ferret primary fibroblasts derived from a male E28 fetus with AV.CFtarg at a multiplicity of infection of 100,000 particles per cells. On day 1 post-infection, fibroblasts were subsequently serially diluted into twenty 96-well plates at 200 to 500 cells per well. These seeding densities allowed about 15 to 30% of wells to give rise to G418-resistant clones. Selection was initiated on day 2 following replating by the addition of 300 μg/mL G418 to the media and cells were cultured for an additional 15 days. Typically, this screening gave rise to about 500 G418-resistant clones, which were subsequently expanded into three replica 96-well plates. Once these replica plates reached confluence, a single plate was used for PCR screening of flanking genomic sequences outside each targeting arm of the vector and anchored sequences within the vector.

Nested PCR screening was then performed for the predicted left side homologous recombination event. Cells in the 96 well plates were directly lysed with 10 μL per well of lysis buffer (50 mM KCL, 1.5 mM MgCl₂, 10 mM Tris, pH 8.5, 0.5% NP40, 0.5% Tween 20) and 1/10 of the cell lysate (1 μL) was used for PCR with the 1^{st} round PCR primer set: F1 (5′-TGGTTTCAAGGGAATGGGGTC-3′; SEQ ID NO:7) and intR1 (5′-AAGCGAAGGAGCAAAGCTGCTA-3′; SEQ ID NO:8). 1/50 of the 1^{st} round PCR products was then used as template for the 2^{nd} round PCR with primers: F2 (5′-GGTGCAGGAGGTGTTTTGTCATAGA-3′; SEQ ID NO:9) and intR2 (5′-GCTAAAGCGCATGCTCCAGACT-3′; SEQ ID NO:10). The positive clones were further confirmed by another nested PCR reaction against the right arm of the integration site using the 1^{st} round primer set: intF1 (5′-CGGACCGCTATCAGGACATAG-3′; SEQ ID NO:11) and R1 (5′-TACGAAATGCAGCAAGCGCC-3′; SEQ ID NO:12); and the 2^{nd} round nested primer set: intF2 (5′-AGGTGTCATTCTATTCTGGGG-3′; SEQ ID NO:13) and R2 (5′-CCCAGGCATCCCTGAAACT-3′; SEQ ID NO:14).

The clones that were PCR positive for both the left and right arms of the predicted integration event were expanded to 24 well plates and when possible, 60 mm plates prior to NT rederival of 21 day embryo fibroblasts. Fibroblasts derived from cloned 21 day embryos were used for isolation of genomic DNA and Southern blot confirmation of the integration event. Genomic DNAs were digested with either MI (which has a unique site in the targeting vector) or BamHI (which does not cut within the targeting vector). Southern blotting was visualized with a P³²-labeled CFTR probe against the 5′ upstream intronic CFTR sequence of the left homologous arm, or a Neo probe against the neomycin cDNA.

Rejuvenating fibroblast lines using SCNT. Rederival of highly proliferative fetal fibroblasts from PCR positive CFTR-gene targeted senescent cells was accomplished using SCNT. Immature oocytes were obtained from sable coat-color Jills mated with vasectomized male ferrets 24 hours prior to collection. To retrieve the oocytes, Jills were euthanized by administration of pentobarbital sodium injection (50-100 mg/kg, i.p). Preovulated follicles were punctured with fine forceps in mPBS (Dulbecco PBS supplemented with 0.1% (w/v) D-glucose, 36 mg/L of pyruvate, and 0.4% (w/v) BSA) to release the cumulus-oocyte complexes (COCs). COCs were cultured in TCM-199+10% FBS+10 IU/mL eCG (equine chorionic gonadotrophin; Sigma G4527)+5 IU/mL of hCG (human chorionic gonadotrophin; Sigma C8554). For enucleation, oocytes were transferred to mPBS medium containing 7.5 μg/mL of cytochalasin B (CB, Sigma C6762) in the micromanipulation chamber. Using Nomarski optics, the first polar body and chromosome spindle were aspirated with a minimal volume of oocyte cytoplasm with a 20 μm (inside diameter, ID) PeizoDrill glass pipette (Humagen™, Charlottesville, Va., USA). A senescent fibroblast (diameter≧40 μm) was inserted into the perivitelline space (PVS) of enucleated oocytes using another pipette (35 μm ID). This larger diameter pipette was specifically needed to accommodate the larger size of senescent fibroblasts. The NT reconstructed embryos were transferred into fusion medium (0.3-0.26 M Mannitol, 0.1 mM MgCl₂, 0.1 mM CaCl₂, 0.5 mM Hepes, 0.01% (w/v) BSA), placed between two parallel electrodes and subjected to an electrical pulse of 1 DC of 180 V/mm for 30 gseconds from an ECM 2001 (BTX, San Diego, Calif.). The fused embryos were then activated with 5 mM ionomycin for 4 minutes, then with 2 mM 6-dimethylaminopurine for 3 hours. Activated embryos were cultured for 24 hours and then were transferred into pseudopregnant albino recipient Jills. A pseudopregnant state was achieved in surrogate albino virgin Jills through mating with a vasectomized albino male 24 hours prior to embryo transfer (ET). At E21 the fetuses were collected and fetal fibroblasts were established as described above except 300 μg/mL G418 was added to the media. Once expanded, these cells were then used for Southern blot screening of the CFTR gene targeting event.

Cloning of CFTR-targeted ferrets by SCNT. The procedure described for rejuvenating fibroblast lines by SCNT was also used for cloning live birth ferrets from Southern blot-confirmed E21 CFTR gene-targeted fibroblasts (CL-B96 clone) with minimal modification. Briefly, the same pipette was used for both enucleating of the oocyte and insertion of fibroblasts into the PVS of enucleated oocytes. All other NT procedures were identical to those described above. The reconstructed embryos were transferred into albino primipara pseudopregnant Jills. If natural birth did not occur at 42 days gestation, the recipients were treated with prostaglandin (Lutalyse, 0.5 mg to 1 mg i.m.) to induce labor. If no kits were delivered within 3 hours, 0.3 mL of oxytocin was subsequently administered to the Jill. If these treatments were unsuccessful to induce labor within 8 hours, a cesarean section was performed. If the recipients failed to produce enough milk to feed kits, the young were fostered onto another Jill when available.

### Results and Discussion

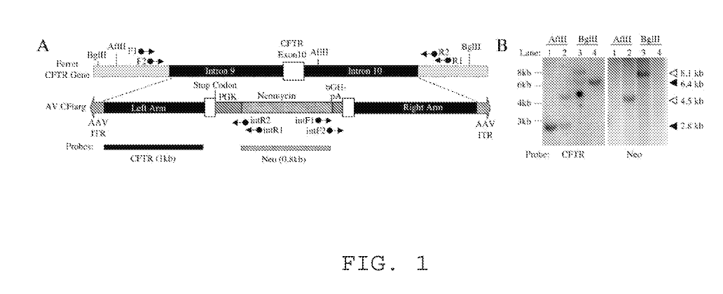

Recombinant AAV-mediated targeting of the CFTR gene in ferret fetal fibroblasts. rAAV has been previously shown to facilitate homologous recombination between its viral DNA and cellular genomic DNA of infected fibroblasts (Russell et al., 1998). Therefore, this virus was chosen to target the CFTR gene in ferret fibroblasts. A ferret BAC library (CHORI-237) was constructed by the BACPAC Resource Center, Children's Hospital Oakland Research Institute, and used to isolate about a 150 kb genomic fragment containing exon 10 of the CFTR gene. Genomic sequences from this BAC clone were used to generate a rAAV targeting vector harboring a PGK promoter driven neomycin resistance gene cassette flanked by sequences encompassing CFTR exon 10 and the adjacent introns (FIG. 1). Male fetal fibroblasts derived from 28 day fetuses were used for CFTR gene targeting with rAAV serotype-2. Fibroblasts were infected with rAAV2 virus at a multiplicity of infection of 100,000 particles per cell and subsequently serially diluted into 96-well plates and placed under G418 selection. During optimization of this procedure, the number of cells seeded into each well and the timing of G418 section post-plating were found to be the most important variables to the subcloning process that targeted 15 to 30% of wells with surviving clones by 15 days. Typically, seeding 200 to 500 cells per well and initiation of selection (300 μg/mL G418) at day 2 following passaging into 96-well plates was optimal.

Following replica plating of each primary 96-well plate of selected fibroblast clones, a single plate was used for PCR screening of flanking sequences outside each targeting arm of the vector (FIG. 1A). In total about 500 clones were typically screened in a single experiment. Although the efficiency of gene targeting ranged from 0.5 to 2% depending on the experiment, the largest hurdle was found to be the rapid senescence of PCR positive targeted clones once they were expanded to 24-well plates. Initially, this was an obstacle that prevented direct confirmation of gene targeting by Southern blotting prior to SCNT.

Rejuvenation of PCR positive CFTR-targeted senescent fibroblasts by SCNT. CFTR-targeted ferret fibroblast clones senesce rapidly during expansion following the initial round of PCR screening; therefore, it was necessary to develop methods for rejuvenating and expanding these candidate CFTR-targeted clones for Southern blot confirmation of the CFTR-targeting events. To this end, several rounds of SCNT were performed on three independent PCR positive CFTR-targeted clones, resulting in successfully cloning of six 21 day ferret fetuses by nuclear transfer (NT) (Table 1). Each of these NT fetuses was dissociated with trypsin and primary fetal fibroblasts were generated under selection with G418. DNA was then derived from each of these primary fibroblast lines and used for Southern blotting. From these cultures, one of the three original senescent fibroblast lines (CL-B96) gave rise to secondary NT-derived fetal fibroblasts with a “clean” CFTR gene-targeting event and no other integrations as shown by Southern blotting (FIG. 1B). The remaining two senescent fibroblast lines gave rise to NT-derived fetal fibroblasts that were neomycin-resistant, but had an insertion of the rAAV vector at a non-homologous site in the genome. NT-derived fetal fibroblasts from the CL-B96 rejuvenated fibroblast clone were expanded to passage 5 for SCNT cloning of live birth ferrets.

TABLE 1
Rejuvenation of senescent CFTR gene-targeted ferret fibroblasts by SCNT

| Senescent | | | | | |
|---|---|---|---|---|---|
| PCR | | | | | |
| positive | | | | No. of | Southern |
| CFTR- | | | | rejuvenated | blot |
| targeted | No. of NT | | No. of 21- | secondary | positive |
| fibroblast | embryos | No. of recipients pregnant | day fetuses | cell lines | for CFTR- |
| clones | transferred | (%) | (%) | (Neo^{R}) | targeting |
| CL-58 | 230 | ⅙ (16.7) | 2 (0.9) | 2 | 0 |
| CL-B96 | 130 | ⅓ (33.3) | 1 (0.8) | 1 | 1 |
| CL-124/590 | 143 | ⅓ (33.3) | 3 (2.1) | 3 | 0 |

SCNT cloning of CFTR-targeted ferrets from fetal fibroblasts. Methods of SCNT cloning of ferrets using cumulous cells have been reported (Li et al., 2006). However, in initial pilot studies, these previous methods were not compatible with efficient cloning from fetal fibroblasts. Thus, ferret SCNT protocols were optimized to resolve the major deficiencies preventing successful ferret cloning with fetal fibroblasts. Major variables included the age of recipient oocytes, alterations to media designed to reduce the stress during embryo manipulation, and optimization of the timing for oocyte activation to more efficiently promote nuclear remodeling. Using this optimized method for ferret fibroblasts, a doubling in oocyte implantation as compared to the previous methods developed for cumulous cells was observed (Table 2). This enhanced level of implantation also led to normal fetal development in 2.2% of implanted reconstructed embryos, whereas fetal development failed to occur using the previous methods used with cumulous cells (Table 2).

TABLE 2
Optimization of SCNT procedures with ferret fibroblasts

| | No. of | No. of | No. of | No. of | No. of | |
|---|---|---|---|---|---|---|
| | oocytes | oocytes | embryos | recipients | implant- | No. of |
| | fused | cleaved | trans- | pregnant | ations | fetuses |
| Procedure | (%) | (%) | ferred | (%) | (%) | (%) |
| Original* | 100 | 81 | 110 | 0/3 (0) | 4 (3.6) | 0 (0) |
| | (90.9) | (73.6) | | | | |
| Optimized^{†} | 262 | 224 | 276 | 4/6 (66.7%) | 18 (6.5) | 6 (2.2) |
| | (94.9) | (81.2) | | | | |

*Methods used for cumulous cell-based SCNT as previously reported (Li et al., 2006).
^{†}Methods described herein for fibroblast-based SCNT.

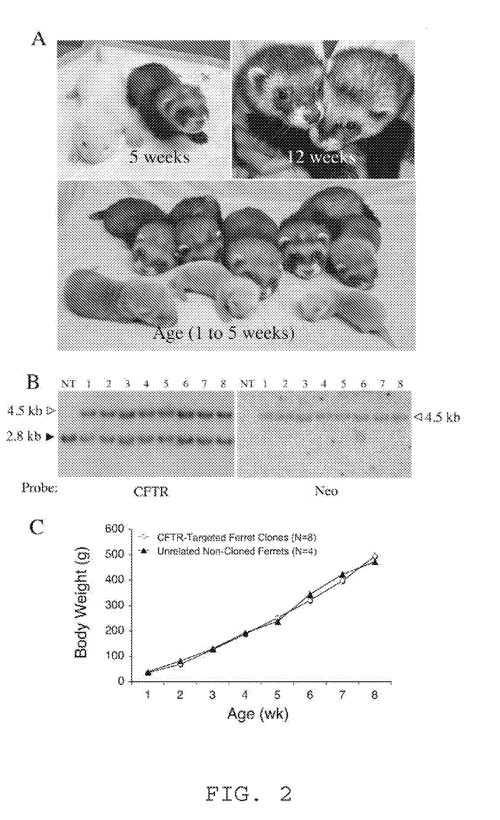

Using this improved method of SCNT with ferret fibroblasts, live birth ferrets were cloned from the NT-rejuvenated, CFTR-targeted fibroblasts (CL-B96). In total, 11 recipient Jills were each adoptively transferred with 35 to 60 reconstructed NT embryos derived from the CL-B96 line and allowed to develop to term (Table 3). Fourteen live pups were born by natural birth or c-section from these 11 Jills. Twelve of these 14 pups appeared to be healthy at birth. In three cases, however, the Jills only nursed one pup in the litter or failed to nurse at all. Two abandoned pups were rescued by transfer to foster Jills. However, it was not possible to do this in all cases of parental neglect since surrogate Jills were not always available. Two of the 14 pups born appeared weak and died; however, it was unclear if the Jills were lactating and surrogates were not available at the time. Southern blotting confirmed that all 8 of the surviving NT cloned ferrets (FIG. 2A) were heterozygous for a single targeted allele of the CFTR gene (FIG. 2B). Furthermore, these ferrets all remain healthy and had preweaning growth rates similar to non-cloned pups (FIG. 2C).

TABLE 3
Cloning of CFTR-targeted ferrets by SCNT.

| | Reconstructed | | | |
|---|---|---|---|---|
| | Oocytes | | Pups | Percent |
| Recipient Jill | Transferred | Live Births | Surviving | Efficiency^{†} |
| 1 | 60 | 1\hat{ } | 1 | 1.7% |
| 2 | 45 | 0 | 0 | 0 |
| 3 | 38 | 2* | 1 | 5.3% |
| 4 | 41 | 2 | 2 | 4.9% |
| 5 | 45 | 1 | 1 | 2.2% |
| 6 | 38 | 1 | 0 | 2.6% |
| 7 | 46 | 3* | 1 | 6.5% |
| 8 | 35 | 1 | 0 | 2.9% |
| 9 | 49 | 2\hat{ } | 2 | 4.1% |
| 10 | 35 | 0 | 0 | 0 |
| 11 | 40 | 1* | 0 | 2.5% |
| Total (N = 11) | 472 | 14 | 8 | 3.0 ± 0.6% |

^{†}Percent efficiency of transferred oocytes that give rise to live births
*Despite the fact all pups were born healthy, the Jill only nursed one pup and neglected others, or failed to nurse all pups in the litter.
\hat{ }One abandoned pup was transferred to a foster Jill.

Conclusions. Combining rAAV-mediated gene targeting with SCNT cloning lays the foundation for numerous other applications in disease modeling with ferrets and other species. Previous reports of generating gene-targeted animals in pig (Lai et al., 2002; Dai et al., 2002), cow (Kuroiwa et al., 2004), and sheep (McCreath et al., 2000) have utilized linear fragments to facilitate gene targeting with non-viral gene transfer methods. Important differences between these previous approaches and the current strategy are worth noting. First, gene targeting in pig and sheep fibroblasts have utilized expression from the endogenous gene target to facilitate positive selection by inserting an internal ribosome entry site upstream to the resistance marker gene. This was not feasible for targeting the CFTR locus since this gene is not expressed in fibroblasts. A second strategy used to target the bovine gene encoding immunoglobulin-μ required an alternative approach since this gene is not expressed in fibroblasts (Kuroiwa et al., 2004). In this context, a diphtheria toxin A gene was used as a negative marker to select against random integration events and led to a targeting efficiency of about 0.5%. This efficiency of targeting appears to be similar to that obtained in the present experiments to target the ferret CFTR allele using rAAV.

Selection of gene-targeted fibroblasts can lead to rapid senescence. This was indeed the case in the ferret where CFTR-targeted fibroblast clones rarely expanded beyond 1×10⁶ cells. Factors affecting senescence appear to be linked to neomycin selection and stress induced by this process, as serial dilution cloning of non-selected fibroblast could easily be expanded to >5×10⁷ cells. This phenomenon may be one reason why there are few reports of gene-targeted animals produced by SCNT. Reversal of this phenotype in fibroblasts by NT, as described herein, indicates that the causes of selection-based senescence are not permanent. Cell rejuvination provides an alternative strategy to more efficiently produce gene-targeted animal models. Interestingly, senescence of CFTR-targeted fibroblasts was not a major obstical in the cloning of CF pig models, suggesting that species-specific factors likely influence the biology of selection-induced senescence.

The creation of both CF ferret and pig models provides new opportunities for dissecting the pathophysiology of CF and testing of new therapies not previously approachable in mouse models of this disease. Furthermore, the fact that AAV-mediated gene targeting was successfully used to genetically engineer both ferret and pig models suggests that this technology may be generally applicable to modeling in any species. Under the proper light cycle, ferrets can reach sexual maturity in approximately 5-6 months.

Hence, with a gestation time of 42 days, CFTR-deficient ferrets could be available in approximately 1-1.5 years. Since ferrets are a preferred model for other devastating infectious human lung diseases, such as H₅N₁ influenza (Subbaro et al., 2007) and SARS (Martha et al., 2003) virus, the ability to generate genetically engineered ferrets may also be of significant utility to pandemic viral disease research.

### Example 2

Development of an efficient cryopreservation technique for the domestic ferret is important for the long-term maintenance of valuable genetic specimens of this species and for the conservation of related endangered species. Unfortunately, current cryopreservation procedures, such as slow-rate freezing and vitrification with open pulled straws, are inefficient. A pipette tip-based vitrification method is described below that significantly improves the development of thawed ferret embryos following embryo transfer (ET).

### Materials and Methods

Chemicals and animals. Chemicals were purchased from Sigma Chemical Co. (St. Louis, Mo., USA) and Invitrogen Co. (Grand Island, N.Y., USA) unless otherwise noted. Ferrets were purchased from Marshall Farms (North Rose, N.Y., USA). Sable and albino coat-color Jills (nullipara, 6-7 months of age, weight 610-851 grams) were in estrus when delivered. Breeder male ferrets (10-12 months of age) were used for mating with Jills for embryo production, and vasectomized males were used to induce pseudopregnancy. Vasectomized males were confirmed as sterile at Marshall Farms by the lack of spermatozoa in ejaculates and the inability to reproduce following several mating attempts. All ferrets were housed in separate cages under controlled temperature (20-22° C.) and a long day light cycle (16 hours light, 8 hours dark). The use of animals in this study was carried out according to a protocol approved by the University of Iowa Institutional Animal Care and Use Committee, and conformed to or exceeded National Institutes of Health standards.

Mating of embryo donors and recipients. Jills with maximal vulval swelling were considered ready for mating. The embryo donor Jills were placed into the breeder male cage for 24 hours and the recipient Jills were mated with a vasectomized male during a 24-hour period. The mating day is recorded as Day 0.

Embryo recovery. Embryos were retrieved from mated donor Jills after the animals were weighed and euthanized at days 4, 5.5 and 6 following the first mating, to collect morula (M, cell number approximately about 8-16 cells), compact morula (CM, cell number approximately 21-32 cells), and early blastocyst (EB, cell number approximately 60 cells) embryos. It should be noted that occasionally both CM- and EB-stage embryos were recovered at day 6. The ovaries, oviducts and uteri of donors were removed and washed with 0.9% (w/v) saline supplemented with 1% (v/v) penicillin-streptomycin (Invitrogen Co.) at 37-38.5° C. Uterine horns and oviducts were flushed with PBS [Dulbecco PBS supplemented with 0.1% (w/v) D-glucose, 36 mg/L pyruvate and 0.4% (w/v) BSA] to release embryos into a Petri dish. Recovered embryos were evaluated under a stereomicroscope (×40) for their developmental stage and photographed.

Vitrification, warming and culture of embryos. The procedure for vitrification and thawing was conducted essentially as described by Piltti et al. (2004) with one key modification; a thin-walled and fine-bored Eppendorf microloader pipette tip was used as the embryo housing vessel. A standard Eppendorf microloader pipette tip (Eppendorf, Cat# 5242-956.003) was cut at a diameter of 0.25 mm. Embryos of all stages were incubated at 37° C. in PBS medium for 1 to 2 minutes, then in cryopreservation medium I (PBS, 20% FCS, 1.34 Methylene glycol, 1.05 M DMSO) for 4 minutes, and finally in cryopreservation medium II (PBS, 20% FCS, 2.95 Methylene glycol, 2.32 M DMSO, 0.9 M sucrose). Two embryos were then aspirated together into an Eppendorf-tip in 0.05 μL cryopreservation medium II. Within 30 to 40 seconds, the tips were immersed in cryotubes filled with liquid nitrogen (LN₂) and stored in LN₂ for 2 to 40 days. Embryos were warmed by immersing the tips at a 30 to 40° angle within 3 seconds after removal from LN₂ into 37° C. PBS medium containing 0.3 M sucrose, and holding them in this position for 1 minute. The embryos were then placed into PBS medium containing 0.15 M sucrose for 5 minutes. After an additional 5 minutes in PBS without sucrose, embryos were washed and cultured in 199 medium containing 10% FCS. The duration of in vitro culture ranged from 2 hours to 2 days. The embryos were then washed, kept in PBS medium on a 38.5° C. warm stage, and transferred into a recipient Jill.

Evaluation of actin cytoskeleton. To visualize the actin cytoskeleton, embryos were fixed overnight in 4% paraformaldehyde, permeabilised by immersion in 0.1% Triton X-100 in PBS for 10 minutes, and then stained for 1 hour at room temperature with a 15 μg/mL solution of Alexa Fluor 488-phalloidin (Molecular Probes Europe BV) in PBS. Labelled embryos were mounted on glass microscope slides with an antifade medium to retard photobleaching (vectashield; Vector Laboratories, Burlingame, Calif., USA), sealed under a coverslip using nail polish, and stored in the dark at room temperature until they were analyzed with Leica confocal laser-scanning microscope. The quality of the actin cytoskeleton was scored as described in Tharasanit et al. (2005) with modifications. A Grade I cytoskeleton was characterized by the precise restriction of actin staining to the cell borders. A Grade II cytoskeleton was characterized by indistinct cell outlines and the presence of small actin clumps in the cytoplasm.

Transfer of vitrified ferret embryos into recipient Jills. A single stock solution of saline containing 10 mg/mL each of ketamine HCl (Abbott Laboratories, N. Chicago, Ill., USA) and xylazine (Phoenix Pharmaceutical Inc., St. Joseph, Mo., USA) was prepared. The recipient ferrets were routinely anaesthetized by i.p. injection of this solution to a final concentration of 20 mg/kg of ketamine and 20 mg/kg xylazine. If the depth of anaesthesia was insufficient, an additional dose of the stock solution was administered, up to a total dose of 30 mg/kg of each drug. During the surgery, a 3 to 4 cm incision was made along the midline of the abdomen to expose the oviducts and uteri. Embryos were transferred into either of the bilateral uteri using a fine glass pipette. The ferret embryo transfer usually required 30 to 60 minutes. After the incision was sutured, the ferrets were returned to their cages and closely monitored until they recovered from anesthesia.

Statistical analysis. Data were analyzed using arcsine transformation and compared by analysis of variance (ANOVA) using Statistics Package for Social Science (SPSS) software. Differences with a P<0.05 were considered significant.

### Results

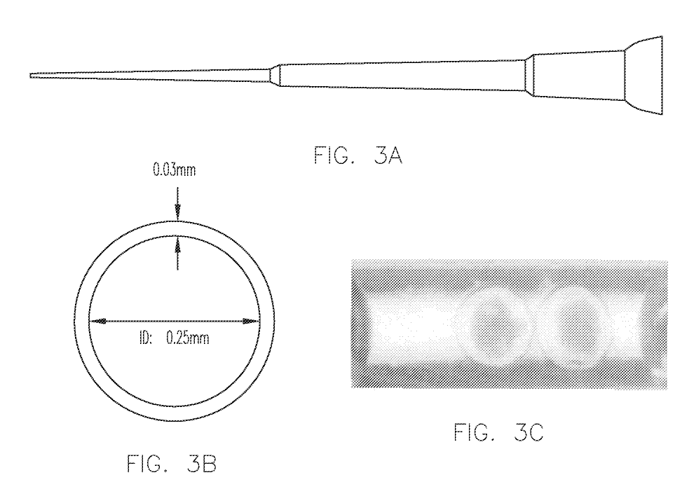

Pipette tip-based vitrification of developing ferret embryos. In an attempt to improve the efficiency of ferret embryo survival and in vivo development following cyropreservation, the vitrification “vessel” was modified. To this end, a modified standard Eppendorf microloader pipette tip was used as the embryo chamber (FIG. 3A). This pipette tip provides a relatively constant inner diameter of 0.25 mm and a uniform wall thickness of 0.03 mm (FIG. 3B). It was reasoned that these physical parameters would be highly suitable in light of both the cooling and warming rates associated with successful vitrification. Two ferret embryos were loaded into each pipette tip (FIG. 3C) and vitrified according to the procedure described above.

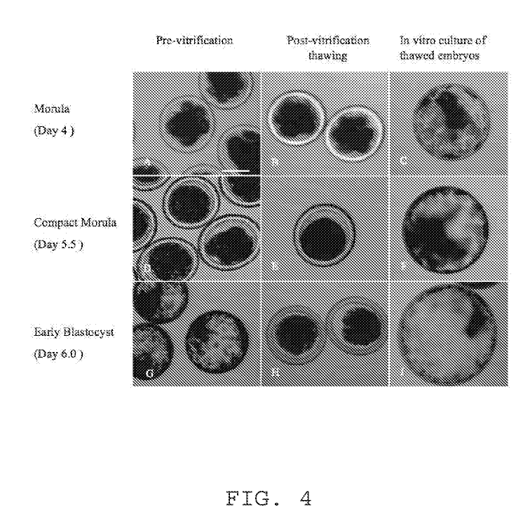

In vitro development of vitrified ferret embryos. Ferret embryos at different stages of development were collected from Jills mated at different times. Morula (M) embryos are shown before vitrification (FIG. 4A), after post-vitrification thawing (FIG. 4B), and at the blastocyst stage following an additional 72 hours of in vitro culture after thawing (FIG. 4C). Compact morula (CM) are also shown before vitrification (FIG. 4D), after post-vitrification thawing (FIG. 4E), and at the blastocyst stage following an additional 48 hours of in vitro culture (FIG. 4F). Finally, early blastocyst (EB) embryos are shown before vitrification (FIG. 4G), after post-vitrification thawing (FIG. 4H), and at the blastocyst stage following an additional 48 hours of in vitro culture (FIG. 4I).

Table 4 compares the rates of blastocyst development among both non-vitrified embryos and embryos vitrified at various stages, following their in vitro culture or thawing and in vitro culture, respectively. In the case of embryos vitrified at the earliest stages (M, 8-16 cell), 62.9% of the vitrified embryos developed to the blastocyst stage whereas significantly more (84.6%) of their non-vitrified counterpart embryos developed to blastocysts under identical conditions. However, this difference appeared only when embryos were vitrified at these early developmental stages; neither embryos that were vitrified at the CM or the EB stage exhibited significant differences in the rates of blastocyst development relative to their control counterparts following in vitro culture (Table 4).

TABLE 4
In vitro development rate of embryos after vitrification

| | | No. (%) of cultured embryos | |
|---|---|---|---|
| Embryo stage | Vitrification | Cultured | Developed to Blastocyst |
| Morula | − | 13 | 11 (84.6)^{a} |
| Morula | + | 27 | 17 (62.9)^{b} |
| Compact morula | − | 10 | 10 (100)^{a} |
| Compact morula | + | 21 | 19 (90.4)^{a} |
| Early blastocyst | − | 10 | 10 (100)^{a} |
| Early blastocyst | + | 9 | 9 (100)^{a} |

^{a,b}Values with different superscripts in the same column differ from each other at P < 0.05.

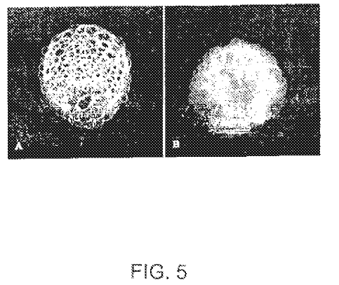

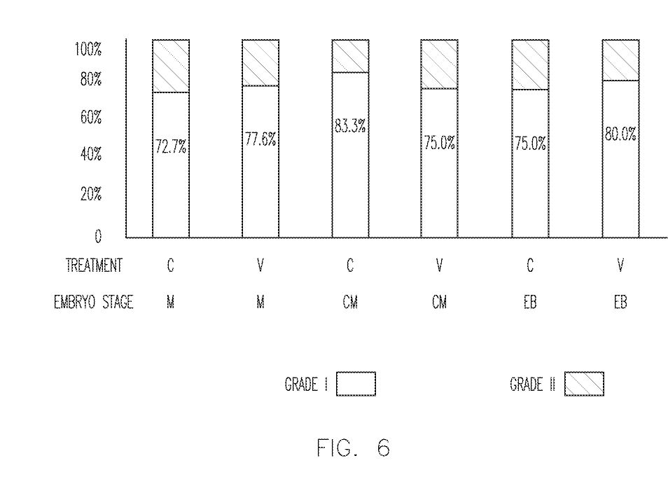

Analysis of the actin cytoskeleton in ferret embryos following vitrification and in vitro culture. An intact cytoskeleton is essential for normal embryo development, and freeze/thaw procedures including vitrification can lead to cytoskelatal damage and poor embryo development following transfer into a recipient female. Assessing the quality of the actin cytoskeleton following vitrification and in vitro embryo culture is, therefore, a good measure of the in vivo developmental potential of vitrified embryos. Embryos with a sharp and distinct network of actin fluorescence surrounding each blastomere following vitrification, thawing, and in vitro culture were thus classified as Grade I (FIG. 5A), whereas identically-treated embryos displaying an abnormal actin cytoskeletal profile (characterized by indistinct fluorescence throughout the embryo, including small clumps of cytoplasmic staining) were classified as Grade II (FIG. 5B).

The actin cytoskeleton was assessed according to these criteria in non-vitrified control (C) and vitrified (V) blastocyst embryos following their in vitro culture (FIG. 6). The percentages of Grade I control (72.7%) and vitrified (77.6%) blastocysts derived from morula embryos were very similar to one another, as were those derived from control and vitrified CM (83.3% vs 75%, respectively) and EB (75% vs 80%, respectively) embryos. Thus the quality of the actin cytoskeleton does not appear to have been adversely affected by this particular vitrification method.

Development of vitrified ferret embryos to term. To determine the rate at which pipet-tip vitrified ferret embryos develop to term following thawing and embryo transfer, vitrified CM- and EB-stage embryos were used in these experiments because of their superior rate of in vitro development relative to vitrified morula embryos (Table 4). Vitrified embryos were thawed and incubated in vitro for short (i.e., 2 hours and 16 hours) or long (i.e., 48 hours and 60 hours) periods prior to embryo transfer. The rationale for varying the incubation time prior to embryo transfer was to allow for embryo acclimation following the potential stress associated with freeze/thawing and for the transfer of embryos at different developmental stages. The interval from the first mating of recipient females to embryo transfer was also varied from 4 days to 7 days, in order to accommodate these differences in the developmental stages of the thawed embryos.

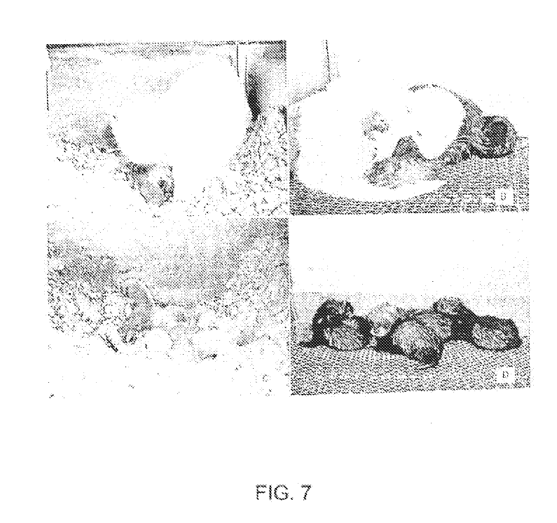

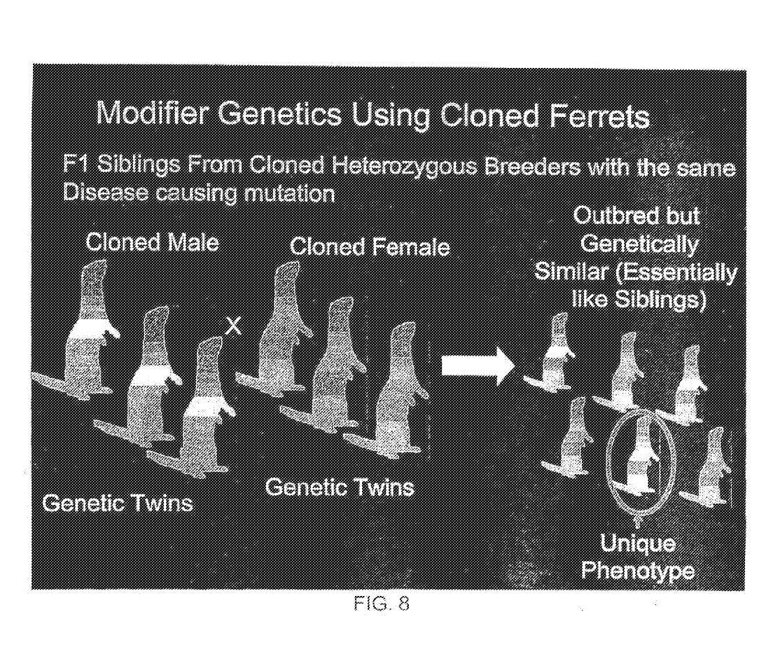

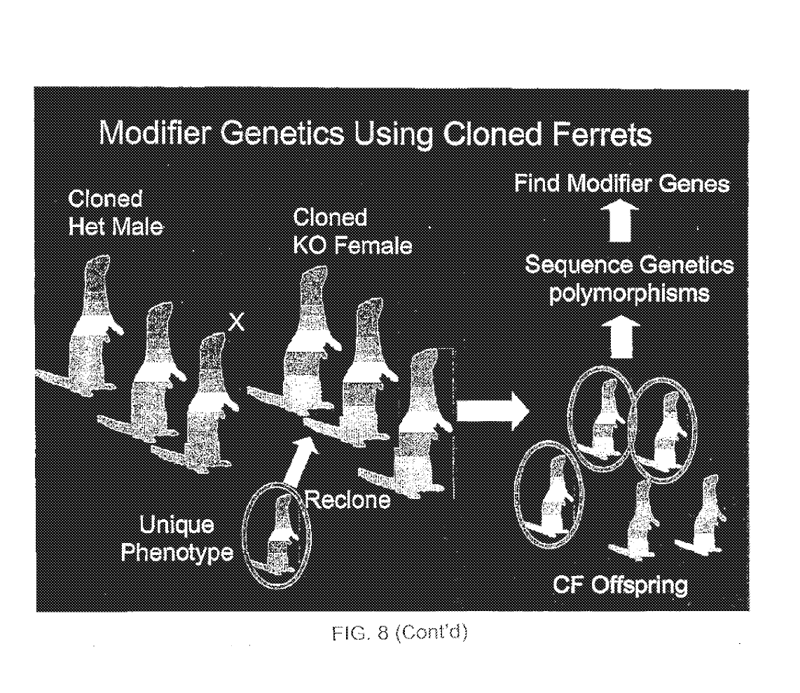

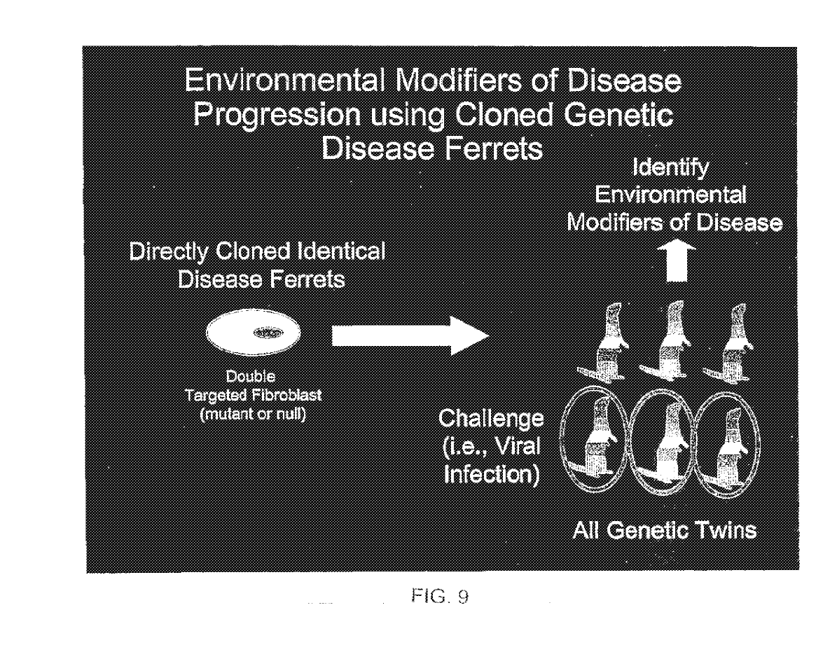

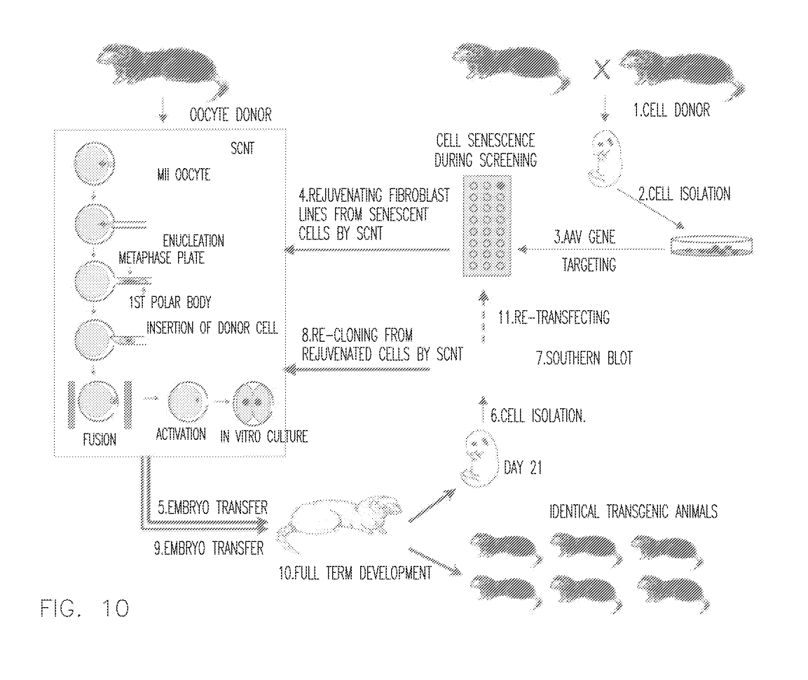

The developmental potential of the vitrified embryos under this range of conditions is summarized in Table 5. This experiment had two important outcomes. First, a short interval between the thawing and transfer of vitrified CM and EB embryos resulted in the highest percentage of live offspring born after 42 days of gestation (i.e., spanning a range of 57.1% to 85.7%). This developmental success was unaffected by the interval between first mating of the recipient and the time of embryo transfer (i.e., across the range of 4 to 7 days post mating). Live ferret pups derived from vitrified embryos are shown in FIG. 7. Sable pups are shown with their albino foster mother 5 days after birth (FIG. 7C) and again at 5 weeks of age (FIGS. 7B and 7D). Second, a long interval between thawing and embryo transfer (i.e., 48 hours and 60 hours) destroyed the viability of the embryos, even though in these cases the recipient mating to implantation interval was correctly matched (i.e., 7-day). Thus, taken together, the data indicate that ferret embryo transfer using the vitrification technique described here is optimal when either CM- or EB-stage embryos are used for vitrification, and the time interval between thawing and transfer of these vitrified embryos is relatively short.

TABLE 5
Effects of culture interval after warming on in vivo development
after transfer of vitrified-warmed CM/EB embryos

| | Embryos Culture | Recipients | No. of | No. of live |
|---|---|---|---|---|
| | interval from | Interval from first | embryos | offspring |
| Interval | warming to transfer | mating to transfer | transferred | (%) |
| Short | 2 hours | 4 days | 8 | 5 (62.5) |
| (2-24 | 2 hours | 4 days | 5 | 4 (80.0) |
| hours) | 2 hours | 5 days | 7 | 6 (85.7) |
| | 2 hours | 7 days | 7 | 4 (57.1) |
| | 16 hours | 7 days | 7 | 6 (85.7) |
| Long | 48 hours | 7 days | 7 | 0 (0.0) |
| (48-72 | 60 hours | 7 days | 8 | 0 (0.0) |
| hours) | | | | |

### Discussion

In the process of cryopreservation by vitrification, the rate of cooling is critical to embryo survival. In earlier methods, a straw containing the embryo(s) was plunged into liquid nitrogen and the cooling rate depended upon three factors: 1) the internal diameter of the straw; 2) the thickness of the straw wall; and 3) the related “holding” volume of medium surrounding the embryo(s). Subsequent development of the OPS technique resulted in a reduction of the straw's inner diameter and its holding volume, thereby, increasing the survival rate of thawed porcine embryos from 18.5% to 29.0% (Berthelot et al., 2003). To increase survival rates even further, a new approach was developed that utilizes a standard micro-pipette tip manufactured by Eppendorf as the vitrification “chamber” (FIG. 3A). This pipette tip chamber differs from the traditional OPS plastic straw in that its inner diameter is narrower (i.e., 0.25 mm versus 0.80 mm) and that the vessel wall is thinner (0.03 mm versus 0.07 mm) (FIG. 3B). These differences in dimension translate into a smaller volume of holding buffer being required (i.e., about 0.05 μL versus about 1 to 2 μL). Moreover, using this new technique, vitrified ferret embryos were efficiently obtained without removing cytoplasmic lipids, a process often required when using the older vitrification techniques for pig embryos containing lipids.

To test the performance of the pipette-tip vitrification technique, the rate of in vitro development of ferret embryos following cryopreservation and thawing was first evaluated (FIG. 4 and Table 4). The majority of vitrified CM (90.4%) and EB (100%) embryos developed to the blastocyst or re-expansion stages following in vitro culture, at rates similar to those for the development of non-vitrified embryos at the same stages. Thus, the data demonstrate that nearly all vitrified ferret embryos at the CM and EB stages are able to develop in vitro following cryopreservation by our pipette-tip technique. By contrast, previous vitrification methods achieved blastocyst re-expansion in only approximately one-half (51%) of either morula- or blastocyst-stage embryos following warming and in vitro culture (Piltti et al., 2004). The data also suggest that ferret embryos at the CM and EB stages are likely more resistant to the cooling damage associated with vitrification than those at the 8-cell and 16-cell stages. These data from ferret appear to be consistent with those obtained from pig, in which embryos at the prehatching stages are more resistant to the effects of freezing than are earlier stage embryos (Dobrinsky, 2001). This difference is presumably due to a reduction in the size of lipid droplets within the pig embryo at more advanced stages (Dobrinsky, 2001). On the other hand, and somewhat surprisingly, expanded ferret blastocysts are not efficiently vitrified when compared with earlier morula- and EB-stage embryos (Lindeberg et al., 2002). However, this difference may relate to the large size of the expanded blastocyst and the resultant need to load a larger volume of medium during vitrification (Lindeberg et al., 2002); a larger volume of medium may reduce the rate of cooling and compromise embryo viability. Taken together, all available data indicate that CM- and EB-stage embryos are best for vitrification in the case of ferret.

It is well known that cryopreservaton can severely disrupt the cellular organization of developing embryos (Dobrinsky, 1996). For example, an intact cytoskeleton is essential for successful mitosis and cell division. When either process is irreversibly compromised, the death of individual cells, or even of the entire embryo, may result. Thus, a successful cryopreservation technique must preserve the integrity of the cytoarchitecture (Dobrinsky, 2001). In the present study, no differences were observed in the appearance of the actin cytoskeleton of post-thawed vitrified ferret embryos and non-vitrified control embryos (FIGS. 5 and 6). These data indicate that the pipette tip technique maintains the integrity of the actin cytoskeleton during and after vitrification. Moreover, the data are consistent with the high rate of live births that was observed following the transfer of vitrified embryos into recipient female ferrets (Table 5 and FIG. 7). Indeed, 25 of 34 (73.5%) vitrified CM and EB embryos were born alive following embryo transfer, which represents a four-fold increase in the live birth rate relative to that achieved using the OPS vitrification method (Piltti et al., 2004). Given that the medium used for cryopreservation in the current study was the same as that used in the earlier OPS study (Piltti et al., 2004), the higher rate of development to term is primarily the result of the pipette tip vitrification technique.

Notably vitrified embryos transferred following longer periods of in vitro culture (i.e., 48 hours and 60 hours) did not develop to term in the present experiments (Table 5). These data are consistent with those in an earlier report on OPS-vitrified ferret embryos (Piltti et al., 2004). The basis for the lack of developmental potential among late-stage vitrified ferret blastocysts is unknown; however, the data herein may provide some important clues. For example, the results show that embryo survival rates are not affected by asynchrony in the developmental stage of the donor embryos and the recipients (Table 5). In addition, it was found that late-stage embryo re-expansion in vitro was compromised irrespective of the vitrification method used (the unpublished data). Thus, it may be that long-term in vitro culture itself is detrimental to embryo survival and that short post-thawing incubation times should be maintained to maximize embryo survival.

Taken together, the data indicate that the pipette tip-based method for vitrification results in a significant improvement in the birth rate of transferred ferret embryos relative to current state-of-the-art methodologies. The novel technique described here should therefore be useful for the long-term maintenance of valuable genetic specimens of Mustelidae, especially in relation to the domestic ferret as a biomedical research model for both human diseases and the conservation of related endangered species.

### Example 3

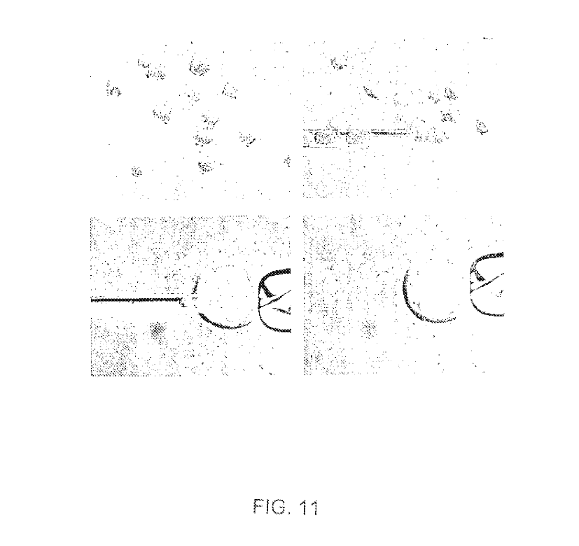

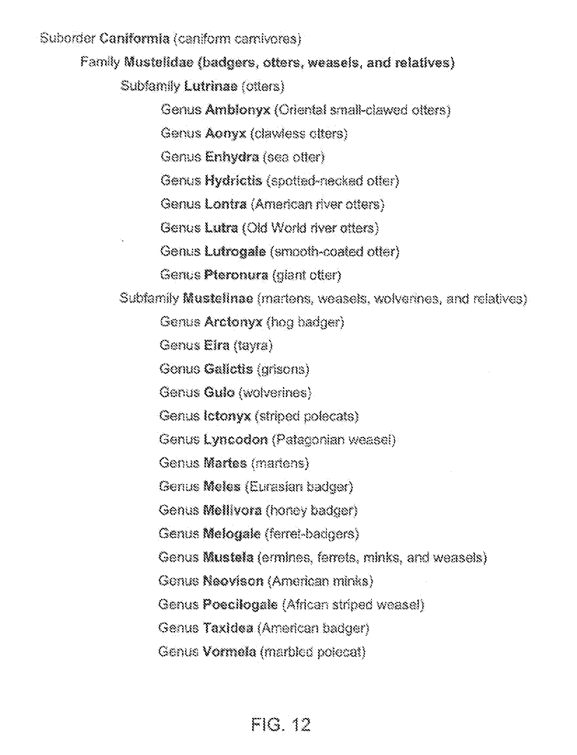

There are a limited number cells obtained when cloning from senescent gene-targeted ferret fibroblast and that limited number of cells is difficult to cryopreserve those cells that results in retention of cell viability, e.g., for later use in somatic cell nuclear transfer. For example, after selection, senescent gene-targeted ferret fibroblast colonies often contain only several thousand cells. To cryopreserve small numbers of gene-targeted cells, cells were cryopreserved in an empty oocyte. For example, empty ferret oocytes were generated from oocytes that were not of sufficient quality for nuclear transfer experiments. The empty oocyte contained an empty zona pellucidae which was generated by micromanipulation in Dulbecco's Phosphate-Buffered Saline using a 130 μm holding pipette and 30 to 40 μm injection pipette. Oocytes were evacuated of all cytoplasmic content by moving the injection pipette through the oolemma (plasma membrane of the oocyte) and injecting medium several times into the oocyte until substantially all of the cytoplasm was pushed out of the oocyte, giving rise to an empty zona pellucidae. Culture dishes with somatic gene-targeted cells were trypsinized and the cells were transferred to a manipulation chamber (FIG. 11A) for aspiration into the injection pipette (FIG. 11B). Cell were then injected into the empty zona pellucidae (FIG. 11C) to create a “pseudoembryo” that contained about 50 to 60 cells (FIG. 11D). The pseudoembryos were then vitrified as described hereinabove and stored under liquid nitrogen. To recover cells, the pseudoembryos were thawed quickly and cells used immediately for nuclear transfer cloning or plated into 96 well dishes. This method allows for small numbers of somatic cells to remain viable during freezing.

### Example 4

Cystic Fibrosis (CF) is the most common life-threatening autosomal recessive condition among Caucasians, with approximately 1 in 3500 newborns affected each year. CF is caused by mutations in an epithelial chloride channel encoded by the cystic fibrosis transmembrane conductance regulator (CFTR) gene (Riordan et al., 1989; Welsch et al., 2001; Rowe et al., 2005). Tissues affected in CF include the lung, pancreas, liver, intestine, gallbladder, sweat glands, and male reproductive tract (Welsch et al., 2001; Rowe et al., 2005). Phenotypic variability in the severity of disease in these tissues can be influenced by modifier genes, the type of CFTR mutation, and the environment, in ways that are only partially understood (Rowe et al., 2005; Drumon et al., 2005). CF mice have been an invaluable system for dissecting the biology of CFTR function, and for demonstrating that genetic background can significantly influence CF-related phenotypes in this species (Egan, 2009; Guilbault et al., 2007). Recently, the description of the neonatal CF pig phenotype has expanded the potential for modeling CF disease (Rogers et al., 2008). Interestingly, CF mice and pigs develop either less or more severe disease in certain organ systems than do CF humans, a fact that highlights species-specific differences in organ physiology and CFTR function. Additional CF models may help to understand how the pleiotropic functions of CFTR in multiple organs influence the progression of lung disease; the most life-threatening aspect of CF being chronic bacterial infections of the airways.

The domestic ferret (Mustela putorius furo) is a potentially attractive species for modeling CF for two major reasons: its lung anatomy and lung-cell biology are similar to those in humans; and it reproduces rapidly (42 days gestation and 4-6 months to sexual maturity). With regard to lung anatomy and lung-cell biology, it is important to note that ferrets and humans have submucosal glands throughout their cartilaginous airways, whereas mice possess these glands only in the proximal trachea (Choi et al., 2000; Robinson et al., 1986). Submucosal glands express abundant CFTR in the serous tubules, which facilitate fluid and mucous secretion into the airway (Engelhardt et al., 1992; Wine et al., 2004); given that these structures are thought to play an important role in protecting the airways from bacterial infection (Wine et al., 2004; Dajani et al., 2005), their distribution may be important for modeling CF disease. Additionally, the goblet cell is the predominant secretory cell type of the human and ferret proximal cartilaginous airways, whereas the Clara cell is the analogous secretory cell type in mice (Robinson et al., 1984; Pack et al., 1980). Although Clara cells are also present in humans and ferret airways, they are limited to the bronchioles rather than being distributed throughout the proximal and distal airways as in mice (Pack et al., 1980; Plopper et al., 1980).

As described below, CFTR-knockout neonatal ferrets developed many of the pathologies observed in CF humans, including meconium ileus (MI), pancreatic disease, vas deferens disease, liver disease, severely impaired nutrition, and a predisposition to lung infections during the early post-natal period. Liver disease in CF ferrets, as evident by a rise in liver function tests during the early post-natal period, was treatable by bile-acid replacement. Additionally, improved nutritional status in the CF ferret model could be achieved by administering an oral proton-pump inhibitor. To complement the gut-defect, a transgenic CFTR-knockout ferret that expresses a HA-tagged fCFTR cDNA under control of the intestinal-specific fatty acid binding protein promoter (FABPi) was generated. The generation gut-corrected CFTR-knockout model lacked MI at birth, and HA-tagged fCFTR was observed in the intestine but not in the lung or liver.

### Methods

Animals and Genotypinq. All animal experimentation was performed according to protocols approved by the Institutional Animal Care and Use Committees of the University of Iowa. Eight CFTR^{+/-} (F0) male ferret clones of sable coat-color background were used to expand the colony by breeding to wild-type sable coat-color jills. F1 CFTR^{+/-} male (hob) and female (jill) offspring from different litters were then crossed to generate F2 litters comprising CFTR^{+/+}, CFTR^{+/-}, and CFTR^{-/-} offspring. Genotyping of F1 ferrets was performed by Southern blotting, using blood drawn at 5 weeks of age and methods as previously described in Sun et al. (2005). In brief, 10 μg genomic DNA was digested with AflII and Southern blotted with an intron-9 α-³²P-labeled probe (Sun et al., 2008). A 2.8 kb fragment represents the wild-type CFTR allele, and a 4.5 kb fragment represents the mutant CFTR allele. A rapid PCR method for genotyping tail clips was developed for F2 offspring as follows. A small piece tail tissue (0.3 to 0.5 cm in length) was lysed in 500 μL lysis buffer (10 mM Tris, pH 8.0, 100 mM NaCl, 1 mM EDTA, 0.5% SDS and 100 μg/mL Proteinase K) at 55° C. for 4 hours. After phenol-chloroform extraction, DNA was precipitated with isopropanol and then rehydrated in 10 mM Tris pH 8.0 buffer. The DNA concentration was adjusted to 0.1 μg/A, and 1 (100 ng DNA) was used in each PCR reaction with 1 μL primer mix and 23 μl AccuPrime Pfx Supermix (Invitrogen). PCR conditions were 95° C. for 5 minutes, followed by 30 cycles of 95° C. for 15 seconds; 55° C. for 15 seconds and 68° C. for 15 seconds. Primer p1 (TGATGATTATGGGAGAGTTGGAGCC; SEQ ID NO:15) and primer p2 (TTGATGGTGCCAGGCATGATCC; SEQ ID NO:16) are complementary to sites upstream and downstream of the target insertion site in exon-10 of the ferret CFTR gene. Amplification using the above-described PCR conditions generated a 100 by DNA fragment from only the wild-type CFTR allele; the 1.9 kb product from the targeted allele was too long to be amplified. The targeted allele was amplified using a third primer p3 (TGGCGGCCGTTACTAGTGGAT; SEQ ID NO:17), which is complementary to sequence within the PGK-Neomycin cassette. The p1/p3 primer set amplified a 180 by product. Primers p1, p2, and p3 were mixed together at a final concentration of 25 pmoles/μl for each primer, and used in the above-described PCR reaction. PCR products (100 by and 180 bp) were resolved on a 1.5% agarose gel.

CFTR immunoprecipitation and phosphorylation assays. Intestine, liver, and lung from newborn ferret kit were harvested, flushed with saline in the case of the intestine, and snap frozen in liquid nitrogen. This tissue was pulverized and lysed on ice in RIPA buffer (150 mM NaCl, 20 mM Tris-HCl, 1% Triton X-100, 0.1% SDS, 0.5% deoxycholate, pH 8.0) containing a protease inhibitor cocktail (Roche) for 30 minutes. The samples were vortexed every 10 minutes during the lysis procedure. The insoluble fraction was then removed by centrifugation (16,100×g, 4° C., 10 minutes). Equal amounts (1 mg) of soluble protein were diluted in 1 mL of RIPA buffer containing 0.1% tween 20, and then pre-cleared (3×) using an irrelevant mouse IgG1 antibody linked to protein G DynaBeads (Invitrogen). This pre-clearing was necessary to prevent bead aggregation during the immunoprecipitation. The soluble protein was immunoprecipitated using a combined cocktail of mouse anti-CFTR antibodies M3A7 and MM13-4 (Chemicon) and anti-HA antibody (Roche), and in vitro phosphorylated as previously described in Ostedgaard et al. (2007). The samples were then resolved on 7.5% SDS-PAGE gels, fixed, dried, exposed to a phosphoscreen, and scanned with a Typhoon 8600 Variable Mode Imager (Molecular Dynamics).

Bioelectric measurement in tracheal xenografts. Functional assessment of transepithelial potential difference (TEPD) was evaluated in CFTR^{+/+}, CFTR^{+/-}, and CFTR^{-/-} tracheal xenografts. The xenograft approach involves cannulating tracheas from newborn kits with flexible tubing, followed by subcutaneously transplantation into nu/nu mice (Zhang et al., 1996). Xenografts were irrigated twice weekly with F12 medium and remained air filled. In this model, the transplanted airways vascularize by 2-3 weeks and are fully differentiated by 5-6 weeks following transplantation. TEPDs were measured using two 1M KCl/5° A, agar electrodes linked to a voltmeter through a set of calomel electrodes within a 1M KCl solution. The positive agar electrode was placed in the luminal tubing of one end of the xenograft, and the negative electrode was placed subcutaneously in the back of the mouse. Xenografts were first irrigated with N-2-hydroxyethylpiperazine-M-2-ethanesulfonic acid (HEPES) phosphate-buffered Ringers solution (HPBR-D) (10 mM HEPES, pH. 7.4, 145 mM NaCl, 5 mM KCl, 1.2 mM MgSO₄, 1.2 mM Ca-gluconate, 2.4 mM K₂HPO₄, 0.4 mM KH₂PO₄, and 100 μM 4,4′-diisothiocyanostilbene-2,2′-disulfonic acid) followed by sequential changes to the following buffers: 1) HPBR-D+100 μM amiloride, 2) Chloride-free HPBR-D (with gluconate replacing Cl⁻)+100 μM amiloride, 3) Buffer 2+200 μM 8-ctp-cAMP, 10 μM forskolin, and 4) Buffer 3+50 μM CFTRᵢₙₕ-GlyH101. Millivolt (mV) recordings were taken by means of a computer-assisted recorder.

Functional measurement of gland secretions in tracheal xenografts. Rates of mucous secretion from single submucosal glands were determined on excised CFTR^{+/+}, CFTR^{+/-}, and CFTR^{-/-} tracheal xenografts, as previously described in Joo et al. (2001), with modification to optimize xenograft viability. Xenografts were harvested and placed in minimal essential medium (MEM) with antibiotics, and shipped overnight at 4° C. from the University of Iowa to Stanford University. Tracheal xenografts were open along the posterior trachealis muscle, and dissected tracheal segments were mounted mucosal-side-up in a chamber without removing the cartilage at 4° C. The tissue surface was blotted dry and then further dried with a gentle stream of 95% O₂/5% CO₂ gas, followed by application of water-saturated mineral oil to the surface. The bath temperature was then gradually increased to minimize potential gland secretions caused by a sudden temperature change. During functional measurement, the tracheal segments were maintained in 37° C. Krebs-Ringer bicarbonate buffer (KRB) bubbled with 95% O₂-5% CO₂ in a humidified atmosphere. The composition of the KRB was 115 mM NaCl, 2.4 mM K₂HPO₄, 0.4 mM KH₂PO₄, 25 mM NaHCO₃, 1.2 mM MgCl₂, 1.2 mM CaCl₂, 10 mM glucose, and 1.0 μM indomethacin (pH 7.4). Following measurements of baseline secretory rates for 20 minutes, three stimulatory conditions were tested sequentially for 30 minutes with the following solutions: 1) serosal (bath) application of 3 μM forskolin alone, 2) serosal application of 3 μM forskolin and 100 nM carbachol, and 3) serosal application of 1 μM carbachol alone. Only the gland secretory rates for stimulatory conditions 1 and 3 are presented in the results. The genotype-dependent trends for stimulatory conditions 2 were intermediate to condition 1 and 3. Condition 2 was tested to evaluate the synergy of cAMP- and Ca²⁺-dependent pathways for glandular secretion that have previously been described in Choi et al. (2007). Digital images of the spherical mucous bubbles under mineral oil were obtained at 5 minute intervals, based on which the secretory rates for individual glands was calculated using ImageJ software (NIH, MD). Typically about 10-15 glands were imaged per xenograft tissue sample. The average number of gland bubbles observed per mm² was 1.8+/−0.7 for non-CF samples, and 1.9+/−0.3 for CF samples. To confirm that differences in glandular secretory rates between CF and non-CF xenografts were not due to differences in gland size, we performed morphometric analysis on the samples analyzed. After functional assays were completed, the xenograft tissue was fixed in formalin and paraffin embedded, and 5-6 sections at approximately 500 μm intervals through the depth of the trachea were obtained. The unit length of the surface airway basal lamina and the glandular area were measured for each section using Metamorph software. The total length of the surface airway epithelium (basal lamina) was then divided by the glandular area for each sample, to produce a glandular size index for each sample. On average about 75 photomicrographs were evaluated for each xenograft sample (N=5 for non-CF and N=6 for CF). Statistical comparison of the glandular size indexes using the student's t-test demonstrated that the glandular size did not differ significantly between the two genotypes.

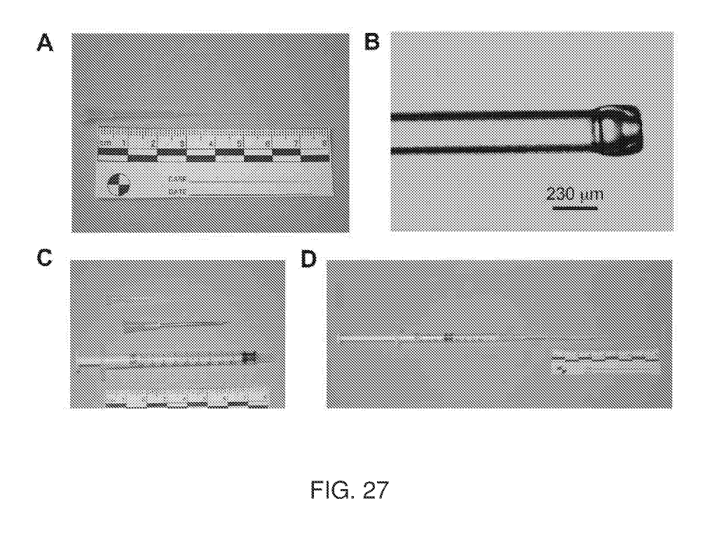

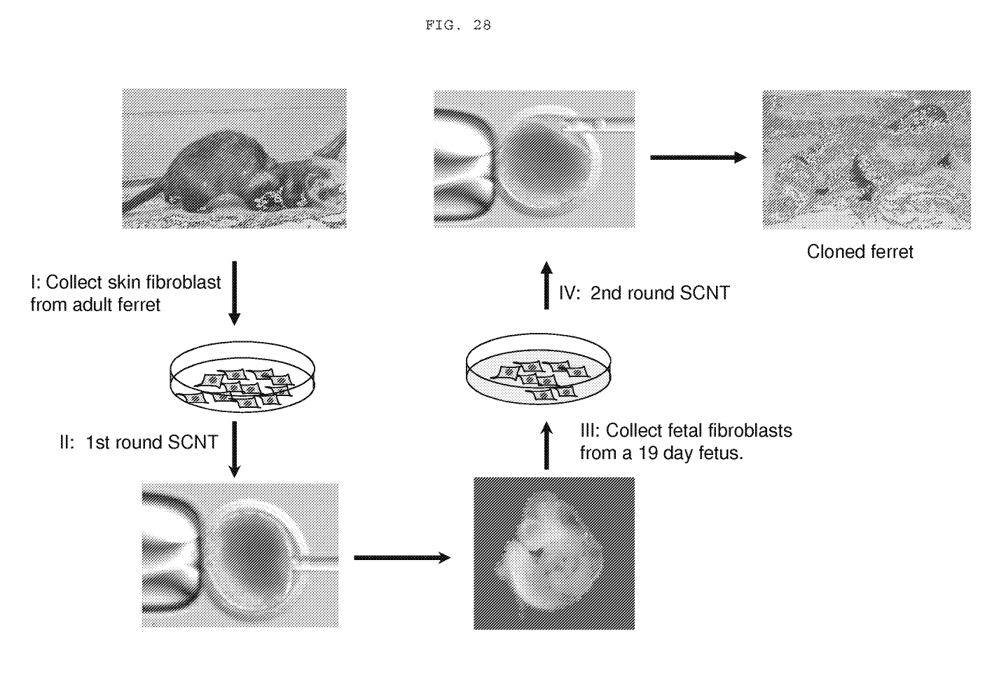

Neonatal clinical care of CFTR^{-/-} kits. Two treatment protocols were developed to enhance post-natal survival and reduce the risk of intestinal obstruction caused by MI. Both protocols included administering metronidazole (5 mg/kg in 100 uL of dextrose saline, s.c.) 4× daily beginning at the time of birth, and starving the animals for the first 3 hours after birth and then administering a 150-200 μL Golytely® gavage (Braintree Laboratories Inc.) using a fire polished microloader pipette tip (Eppendorf Inc.) (FIG. 27 for example of gavage tube components). The gavage was repeated every 6 hours up to three times, until meconium was passed. After the CF kits passed meconium they were fed, by gavage, 150-200 μL Elecare (Abbott Laboratories) supplemented with UDCA (5 mg/kg) and/or omeprazole (5 mg/kg) 4× daily. When the diet was supplemented with pancreatic enzymes, Viokase-V was used and dosed according to lipase units as specified. Once genotypes were known, typically at 12-18 hours after birth, the litter size was reduced such that only the CFTR^{-/-} kits and an equal number of controls (CFTR^{+/+} and/or CFTR^{+/-}) were kept. When CFTR^{-/-} kits were weaned at 5 weeks of age, they were fed a slurry of Elecare hydrated solid chow (Marshall Farms) 2× daily, mixed with 1 lipase Unit per 10 gram body weight of Viokase-V (the amount of food eaten with each feeding was recorded for these calculations).

Blood chemistries. Blood chemistries were performed using a VetScan VS2 (Abaxis) using blood drawn at the time of clinical death and necropsy. Mammalian Liver Profile rotors from Abaxis were used, and assessed plasma levels of: alkaline phosphatase (AP), alanine aminotransferase (ALT), bile acids (BA), total bilirubin (TBiI), total cholesterol (CHOL), gamma glutamyl transferase (GGT), and blood urea nitrogen (BUN). Indirect and direct bilirubin levels in the plasma were measured using two kits from Diazyme Inc for total bilirubin (DZ150A-K) and direct bilirubin (DZ151A-K), where the indirect bilirubin=total bilirubin−direct bilirubin.

Histopathology. Standard histopathology analysis was performed on paraffin sections from formalin-fixed tissues. Tissues were collected at the time of euthanasia (clinical death) and immediately placed in 10% neutral buffered formalin for at least 72 hours. Tissues were then paraffin-embedded, sectioned (4-5 μm) and stained with hematoxylin and eosin (H&E) or periodic acid-Schiff (PAS). A modified Gram-Twort procedure was used to identify Gram-positive and Gram-negative bacteria in tissue sections (Ollett, 1947). Briefly, this technique uses a Gram stain protocol followed by application of Twort solution (containing nuclear fast red). This procedure results in Gram-positive bacteria staining blue/black and Gram-negative bacteria staining red/pink. Histopathological examination was performed by a veterinary pathologist, and age matched CFTR^{-/-} and CFTR^{+/+;+/-} controls were used.

Bacteriology of bronchioalveolar lavage (BAL) fluid and fecal samples. BAL was collected when animals were euthanized and performed in a laminar flow hood under sterile surgical conditions. A small incision was made to expose trachea and a 23 gauge angiocatheter was inserted into the proximal end of the trachea and stabilized using surgical sutures. 1-2 mL of sterile saline was used to lavage the lung and this was repeated three times and BAL fluid was pooled. Fecal samples were collected from the oldest CFTR^{-/-} and control animal and 0.2 grams was resuspended in 3 mls of sterile saline by vortexing with glass beads. 3% of each BAL fluid or fecal sample was plated onto various types of bacterial growth media for bacteriologies. Fecal and BAL samples were streaked directly and from enrichment broth onto selective and non-selective agar media (Blood agar, Anaerobic Blood agar, MacConkey agar, CNA agar [Colistin and Naladixic Acid], Anaerobic CNA Reducible agar, Chocolate Agar (for BAL only), Hectoen enteric agar (for fecal only), Brilliant Green agar (for fecal only), XLT4 agar (for fecal only), Tetrathionate both, Chopped Meat Glucose broth, Brain Heart Infusion broth). Cultures were incubated aerobically and anaerobically for 24-48 hours at 35° C. All plates were examined for bacterial growth and visually assessed for bacterial types and quantity. Growth was measured semi-quantitatively based on colony distribution on the plates within the four-quadrant streak pattern. Growth on non-selective media was ranked for growth based on the occurrence of colonies quadrant 1-4 (++++), 1-3 (+++), 1-2 (++), or 1 only (+). Representative colonies from mixed cultures were subcultured to purity onto appropriate media. Bacterial identification was based on colony morphology, gram stain, and biochemical reactions (Quinn, 1994). Extended bacterial identification was performed if needed using an API 20E test kit.

Somatic cell nuclear transfer cloning of a CFTR^{-/-} transgenic ferret with intestine-specific expression of wild-type ferret CFTR. A transgene cassette containing the rat Intestinal Fatty Acid Binding Protein (FABPi) promoter (Zhou et al., 1994) (kind gift of Dr. Jeffrey Whitsett, University of Cincinnati, Cincinnati, Ohio) driving the expression of a HA-tagged fCFTR cDNA with a bovine growth hormone poly-A was generated in plasmid pcDNA3.1. This cassette also contained a floxed PGK promoter-driven Zeocin/SV40-poly-A resistance gene cassette for selection of transfected cells. The wild-type fCFTR cDNA was engineered to contain a 3×HA tag in extracellular loop 4 by PCR-mediated cloning, and its sequence was based on that previously described for generating 3×HA-tagged human CFTR (Glozman et al., 2009). The cDNA sequence of the 3×HA-tag was 5′-agtactcagagcCTCGAGTACCCTTACGACGTTCCT GATTACGCTGCTAGCTACCCTTACGACGTTCCTGATTACGCTTACCCTTACGACGTTCCTGATTACGCTG CTTCTataaatagcagt-3′ (SEQ ID NO:18; sequences in lower case show homology to the junctional fCFTR sequence, and those capitalized represent the 3×HA-tag sequence. The amino acid sequence of this tag is as follows: STQSLEYPYDVPDYAASYPYDVPDYAYPYDVPDY AASINSS (SEQ ID NO:19; italicized amino acids are junctional fCFTR amino acids, and non-italicized amino acids are the HA-tag sequence; the underlined letters are the three HA amino acid epitopes). The linear fragment encompassing the FABPi-HA-fCFTR/PGK-Zeocin transgenic cassette was isolated by Pmel digestion of the pFABPi-HA-f CFTR-Zeo plasmid, followed by agarose-gel purification. This gel-purified fragment was then transfected into primary fibroblasts generated from a female 28-day old CFTR^{-/-} kit. The transfected CFTR^{-/-} fibroblasts were selected in 50 μg/mL of zeocin (Invitrogen) for 3 weeks, and the zeocin-resistant cells from this pool were used as donors for SCNT according to previously described protocols (Sun et al., 2008; Li et al., 2006). Cloned kits were evaluated for the presence of the transgene in their genomic DNA using PCR and phenotyped for MI and tissue expression of the HA-f CFTR transgene.

Statistical analyses. Statistical significance for all comparisons (with the exception of inheritance studies) was assessed using an unpaired, two-tailed Student t-test. Inheritance of the MI phenotype was performed using the Pearson's chi-square test, Fisher's exact test, and logistic regression. For logistic regression, the response variable was the MI status of the offspring and the explanatory variable was the hob and/or Jill identification. In all statistical analyses, p-values<0.05 were deemed significant.

### Results

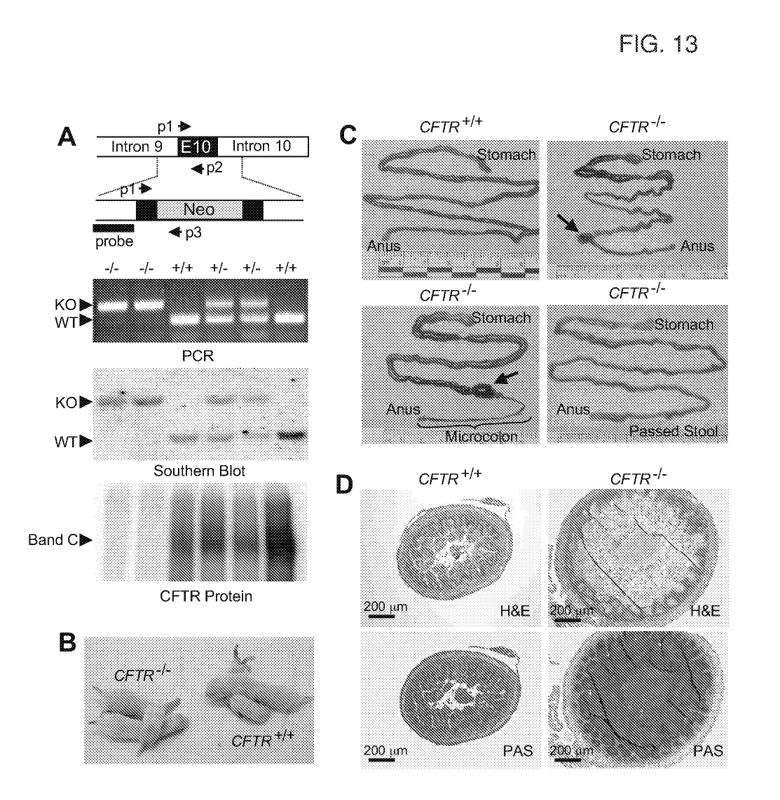

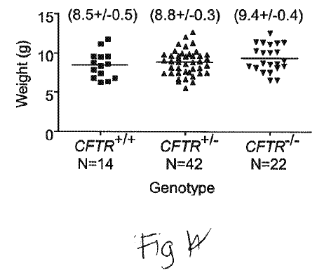

CFTR-knockout kits exhibit meconium ileus of variable penetrance. Eight heterozygous founder clones (CFTR^{+/-}) were expanded through a single generation of breeding, and their F1 CFTR^{+/-} offspring gave rise to 313 kits containing 65 CFTR^{+/+}, 171 CFTR^{+/-}, and 77 CFTR^{-/-} animals (FIG. 13A). This ratio was not significantly different from the expected 1:2:1 inheritance of a recessive trait, and indicates that prenatal lethality is not a consequence of deleting the CFTR gene in ferrets. Intestinal tissue from CFTR^{-/-} kits expressed no CFTR protein as compared to CFTR^{+/+} and CFTR^{+/-} kits (FIG. 13A). Within the first 24 hours of life, CFTR^{-/-} kits were indistinguishable from CFTR^{+/+} or CFTR^{+/-} littermates in terms of activity and weight (FIG. 13B and FIG. 14). However, by 36 hours after birth all CFTR^{-/-} kits failed to thrive, and by 48 hours they were typically euthanized due to severe morbidity. Gross and histological pathologies indicated that the majority of kits (about 75%) suffered from meconium ileus (MI) and failed to pass stool during the neonatal period (FIG. 13C). Approximately half of these kits with MI died from intestinal perforation at the level of the ileum or colon (FIG. 13C arrows). Additionally, microcolon was observed in about 30% of the animals born with MI (FIG. 13C). Interestingly, about 25% of newborn CFTR^{-/-} kits passed meconium but nevertheless failed to thrive, and died within 2-4 days after birth (FIG. 13C). Gastrointestinal obstruction in CFTR^{-/-} kits suffering from MI was associated with intestinal luminal mucus and mucous cell hyperplasia (FIG. 13D). The occurrence of MI in ferret CFTR^{-/-} kits (about 75%) was greater than that for CF infants (about 15%) (Welsh et al., 2001; Oppenheimer et al., 1973), and less than that seen in CFTR^{-/-} pigs (100%) (Rogers et al., 2005). Although CF mice do not present with classical MI at birth, they do suffer from gut obstruction at weaning, and this can significantly impair survival. Interestingly, the penetrance of gut obstruction in CF mice can vary significantly (0-95% survival at weaning) depending on the background strain (Egan, 2009; Guilbault et al., 2007), suggesting that genetic modifier genes significantly influence this phenotype in the mouse.

Analysis of the frequency of MI within the CF ferret colony suggested genetic influences for the development of severe intestinal complications at birth in CFTR^{-/-} kits. Nine F2-generation male CFTR^{+/-} hobs used for breeding gave rise to litters with MI frequencies ranging from 50-100% (Table 6). Pearson's chi-square and Fisher's exact tests for association demonstrated a statistically significant dependence of variable MI frequency in CF offspring on the CFTR^{+/-} hobs used for breeding (p<0.047 for both tests). Additionally, a logistic regression was run where the response variable was the MI status of the offspring and the explanatory variable was the hob identification. This analysis also suggested that inheritance of the MI phenotype was influenced by the hob (p<0.042). Inclusion of the female parents (i.e., jills) as an explanatory variable did not reach significance in this logistic regression, since the number of CF kits born to each of the 40 jills used in this analysis was too small, and only 8 of the jills were bred twice. These findings suggest that hereditary factors influence the occurrence of MI in CFTR^{-/-} kits as it does in human CF patients.

TABLE 6
Frequency of MI in CFTR^{-/-} kits born to nine CFTR^{+/-} hobs.

| Hob ID | 910078S | 910288S | 910448S | 910468S | 910518S | 910538S | 910598S | 910608S | 910618S |
|---|---|---|---|---|---|---|---|---|---|
| CFTR^{-/-} kits | 3 | 11 | 3 | 10 | 12 | 14 | 11 | 6 | 12 |
| with MI | | | | | | | | | |
| CFTR^{-/-} kits | 3 | 1 | 3 | 0 | 2 | 1 | 4 | 0 | 5 |
| without MI | | | | | | | | | |
| Total Kits | 6 | 12 | 6 | 10 | 14 | 15 | 15 | 6 | 17 |
| born to hob | | | | | | | | | |
| % MI | 50% | 92% | 50% | 100% | 86% | 93% | 73% | 100% | 71% |

The above data were derived from 56 litters born from 40 jills bred randomly with 9 hobs.

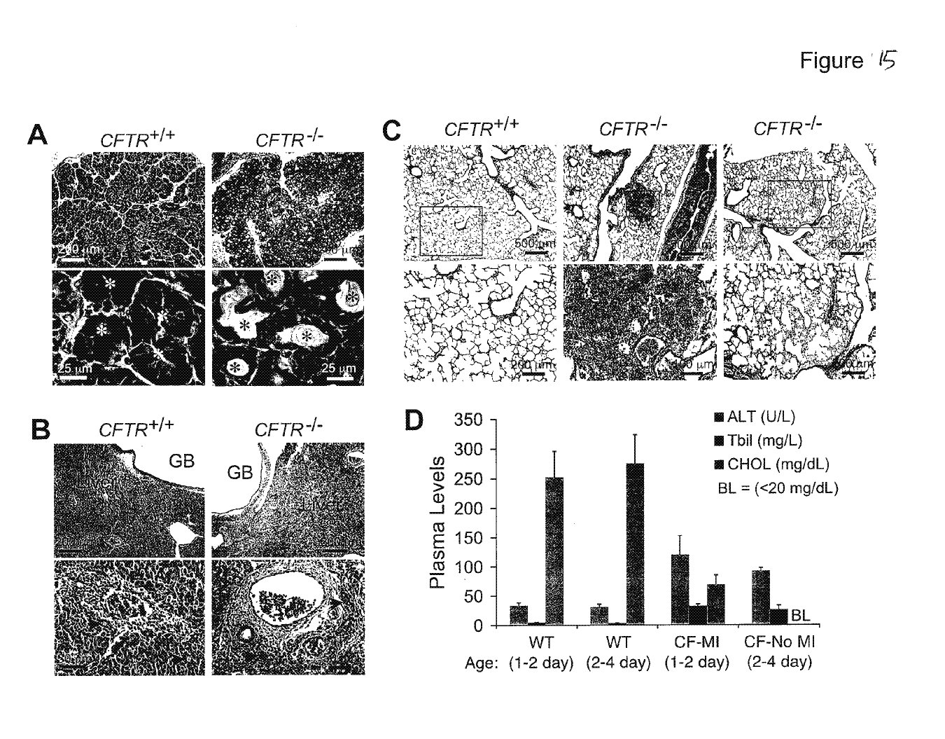

CFTR-knockout kits exhibit pancreatic lesions similar to those seen in CF infants, but no overt histopathology of the liver or gallbladder. The newborn CFTR^{-/-} pancreas was indistinguishable from CFTR^{+/++/-} kits at the gross level, but histologic lesions were evident in all animals. Most, but not all acini and ductules were dilated with inspissated, eosinophilic zymogen secretions (FIG. 15A). Microscopic changes in the pancreas are seen in approximately 75% of CF infants, with the majority of these cases (about 72%) demonstrating histopathology similar to that seen in newborn CFTR^{-/-} ferrets, and a small minority of cases (about 3%) demonstrating more severe lesions, with evidence of loss of exocrine tissue and the development of fibrosis (Oppenheimer et al., 1973; Oppenheimer et al., 1975). Thus, the level of histopathology in the newborn CFTR^{-/-} ferret pancreas appears quite similar to that seen in CF infants, and significantly less severe than the extensive destruction observed in the exocrine pancreas of newborn CF pigs (Rogers et al., 2008). The liver and gallbladder of newborn CFTR^{-/-} kits were indistinguishable from those of CFTR^{+/+} or CFTR^{+/-} littermates at the gross and histological levels (FIG. 15B).

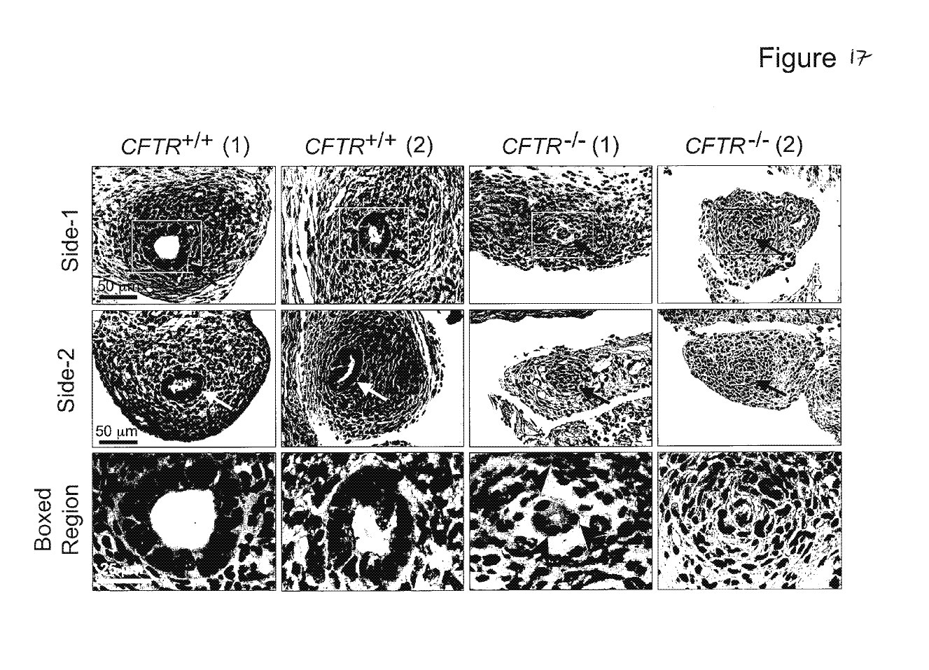

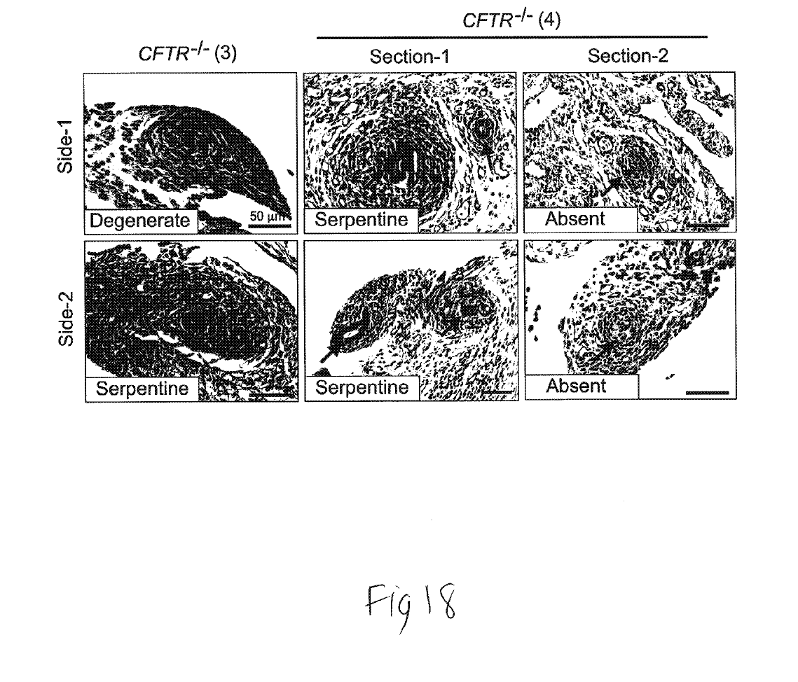

CFTR-knockout kits have a degenerate or absent vas deferens at birth. Nearly all CF male adults suffer from infertility caused by bilateral absence of the vas deferens (Jarzabek et al., 2004). Most CF males have an intact vas deferens at birth, however, mucoid obstruction of the vas deferens have been noted in CF newborns and unilateral absence of the vas deferens as been detected as early as 2 years of age (Oppenheimer et al., 1975). These findings have led to the prominent hypothesis that obstruction of the vas deferens and/or altered secretions lead to progressive degeneration in CF patients (Gaillad et al., 1997). Histopathologic examination of the vas deferens in newborn CFTR^{+/+;+/-} and CFTR^{-/-} littermates demonstrated considerable pathology in the newborn CF kits. Of the five CFTR^{-/-} kits evaluated, the vas deferens was completely absent in about 50% of the spermatic cords (FIG. 17). In the remainder, the vas deferens was segmentally absent with remnant epithelium characterized as small and degenerate to tortuous and serpentine (FIG. 18). In one of the five CFTR^{-/-} kits, there was complete bilateral absence of the vas deferens, while a normal vas deferens was consistently detected in all of the five control CFTR^{+/++/-} kits.

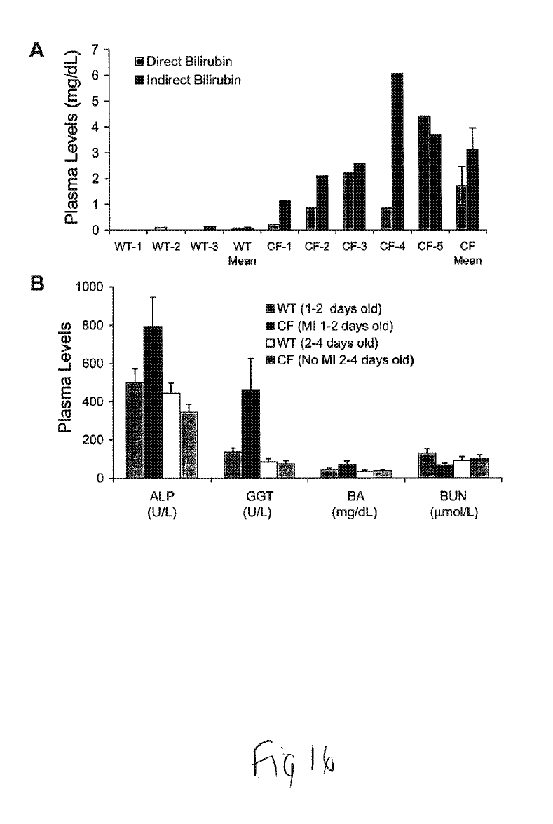

Newborn CFTR-knockout kits that escape MI fail to thrive, and exhibit early lung infections and elevated liver function tests (LFTs). Histopathologic analysis failed to ascertain the reason for early neonatal death of the 25% of CFTR^{-/-} kits that passed meconium. Although the intestine appeared histologically normal in these animals, we noted evidence of bronchopulmonary pneumonia and aspiration (FIG. 15C), which suggested that perhaps CFTR^{-/-} kits were predisposed to aspiration-induced secondary infections. Blood chemistries in this subset of animals revealed that both plasma alanine aminotransferase (ALT) and bilirubin were consistently elevated while cholesterol levels were reduced (FIG. 15D and FIG. 16); these findings suggested the potential for liver disease. Although clinically apparent liver disease in CF patients 1-5 years of age is low (0.3%) (Scott-Jupp et al., 1991), 53% of CF infants demonstrate abnormally elevated liver function tests (LFTs), a feature that typically resolves itself by 2-3 years of age (Lindblad et al., 1999). Interestingly, these neonatal elevations in the levels of serum liver enzymes such as ALT were not associated with neonatal cholestasis when liver biopsies were evaluated (Lindblad et al., 1999). Such findings are similar to those seen in newborn CFTR^{-/-} kits, which demonstrated no histopathologic lesions in the liver despite elevated LFTs (FIG. 15B).

The reason for elevated LFTs in the majority of CF children without histological signs of cholestasis remains unclear (Lindblad et al., 1999). However, CF children frequently exhibit bile acid malabsorption by the intestine, which leads to altered enterohepatic circulation and hepatobiliary composition of bile acids (Harries et al., 1959; Weber et al., 1973; Weber et al., 1985). Excessive fecal bile acid loss has been proposed to influence fat and cholesterol absorption by the gut, bacterial flora in the gut, and the progression of liver disease, through alterations in bile acid pool composition (Weber et al., 1985). Bile acids are synthesized from cholesterol by hepatocytes and transported into bile ducts in a conjugated form (Hofmann, 2009). The present findings of reduced serum cholesterol levels in 2-4 day old CFTR^{-/-} kits that escaped MI are consistent with impaired bile acid absorption by the gut and the depletion of cholesterol pools through defective fat absorption by the gut. Oral administration of the hydrophilic dihydroxylated bile acid ursodeoxycholic acid (UDCA) has been shown to normalize LFTs (including serum ALT and bilirubin) and to improve the nutritional status in CF patients (Cotting et al., 1990; Scher et al., 1995).

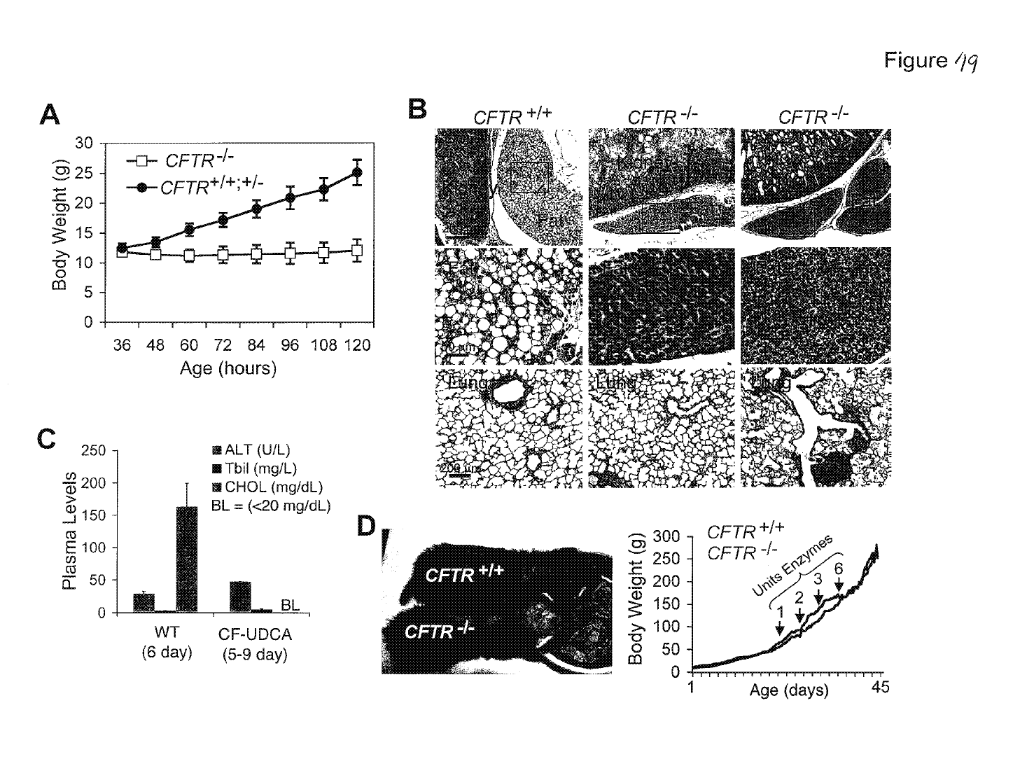

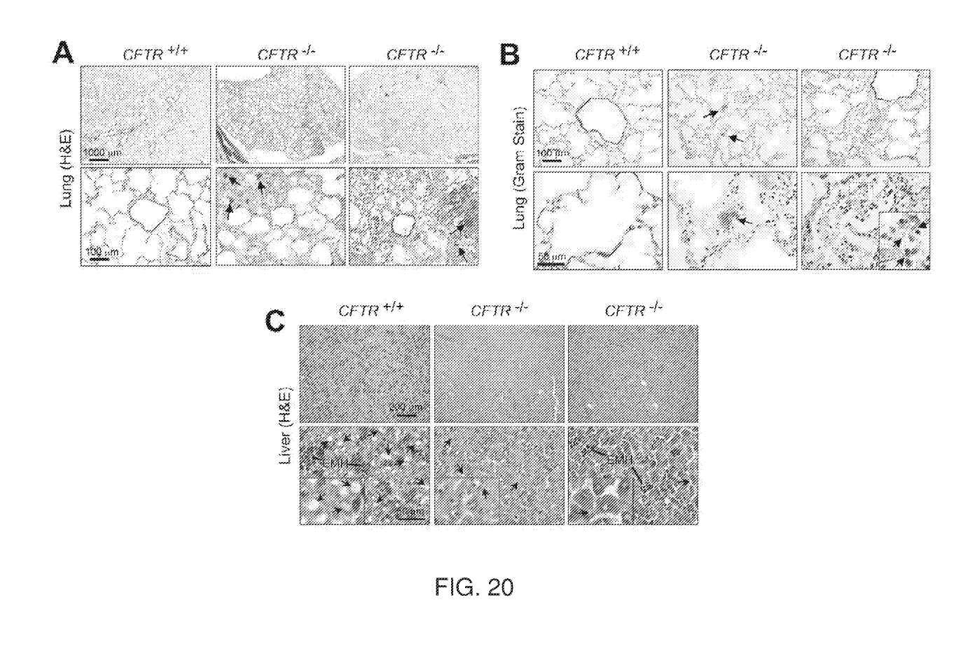

For these reasons, it was determined whether oral UDCA therapy of CFTR^{-/-} kits might normalize liver function and improve nutrition. Additionally, CFTR^{-/-} kits were gavaged with Golytely® within the first 12 hours to enhance rapid meconium clearance, and treated with antibiotics to prevent secondary lung infection during the early neonatal period. Among 20 CFTR^{-/-} kits treated in this manner, 16 died of MI and 4 survived for 5-9 days but failed to gain weight (FIG. 19A). Gross necropsy demonstrated that although there was no intestinal obstruction in the CFTR^{-/-} kits, fat stores throughout the animals were depleted, consistent with sustained malnutrition (FIG. 19B and FIG. 20C). UDCA treatment normalized serum ALT and bilirubin levels, as is the case in CF infants (Scher et al., 1997), but failed to normalize serum cholesterol levels, which remained low in comparison to treated controls (FIG. 19C). Three of the four CFTR^{-/-} kits developed respiratory distress and showed signs of multifocal bronchopneumonia with the lungs demonstrating intralesional neutrophils, macrophages, hemorrhage, fibrin and bacterial colonies (FIG. 14B and FIG. 20). Airways and alveoli were patent and showed no evidence of inflammation, hemorrhage, or bacterial colonization in control-treated CFTR^{+/+} and CFTR^{+/-} kits. Although control lungs were free of pathology, it is currently unclear if lung lesions in the CFTR^{-/-} kit were due to the inability of the weakened animals (i.e., nutritionally compromised) to eradicate the bacteria associated with aspirated material. In a small subset of CFTR^{-/-} kits (N=3) that passed meconium, we also attempted pancreatic enzyme replacement, but this failed to improve either the nutritional status or the survival of these animals.

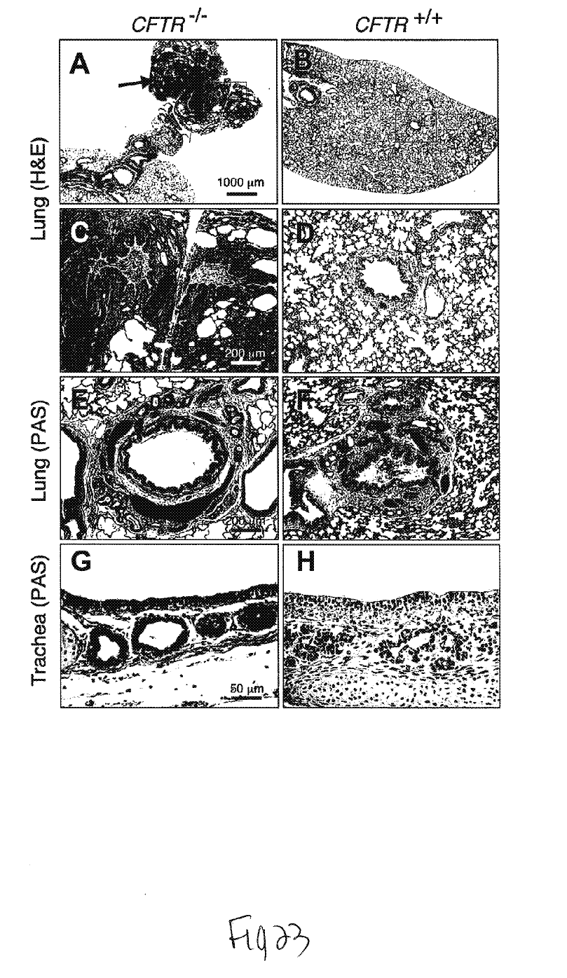

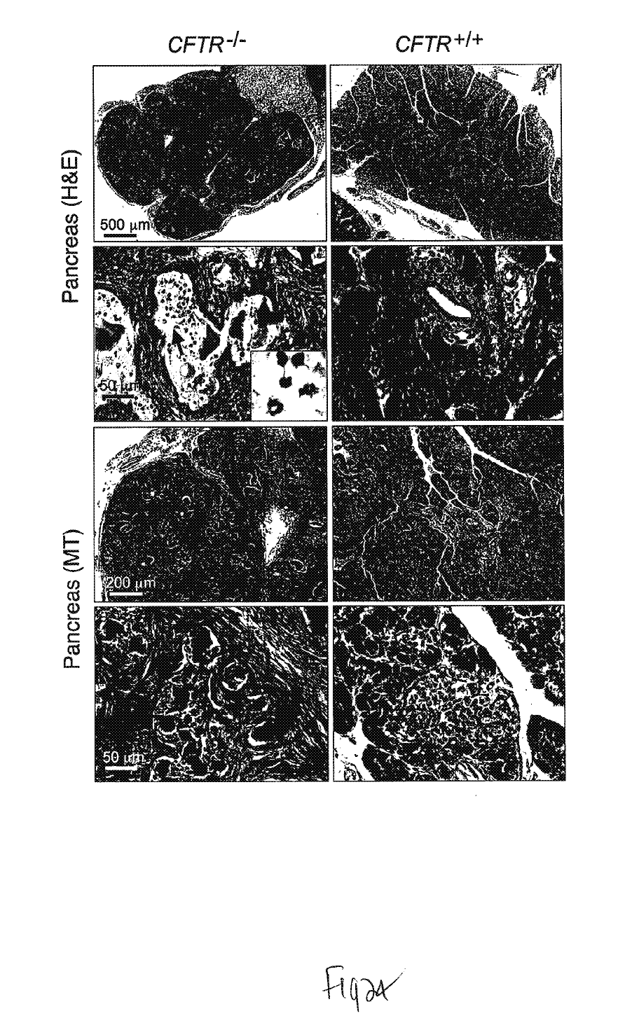

Oral administration of a proton-pump inhibitor improves nutrition in the CFTR-knockout model. Bile acids play an important role in intestinal lipid digestion and absorption, cholesterol homeostasis, and excretion of lipid-soluble xenobiotics (Hofmann; 2004). The solubilization and reabsorption of bile acids by the gut can also be influenced by fecal pH (Hofmann et al., 1992). Given that gut pH would likely be reduced in the context of CF, due to impaired pancreatic secretion of bicarbonate, we reasoned that raising the gut pH by oral administration of the proton pump blocker omeprazole might allow for better bile acid-mediated absorption of fat in the gut, and thereby improve the nutritional status of CF kits. Omeprazole has been used to enhance the function of pancreatic enzyme supplementation in CF patients with residual steatorrhea, to improve fat absorption (Proesmans et al., 2003). Indeed, oral administration UDCA- and an omeprazole-containing liquid elemental diet led to significantly improved weight gain in a CFTR^{-/-} kit during the first 16 days of treatment; weight gain was comparable to that of a CFTR^{+/+} littermate control that was treated identically. However, at 16 days the weight gain of the CFTR^{-/-} kit began to slow in comparison to that of the control, and thus the animals were supplemented with oral pancreatic enzymes. After several weeks of increasing enzyme intake, the CFTR^{-/-} kit recovered weight to nearly match that of the control kit (FIG. 19D). Four additional CFTR^{-/-} kits have been reared using this same approach and also had improved weight gain during the neonatal period, although in these four cases weight gain was only about 25-50% of controls (Table 7). One of these CFTR^{-/-} kits died acutely at 20 days due to a massive aspiration while nursing. The second CFTR^{-/-} kit died at 32 days due to a rectal prolapse (which also occurs in human CF patients). The third and fourth CFTR^{-/-} kits died at 7 and 13 day of age. The cause of death was undetermined in the 7 day kit, but it did have a small focus of pneumonia; and the 13 day kit had intestinal obstruction noted during pathology examination. Histopathologic examination of these animals demonstrated increased pancreatic inflammation and loss of exocrine tissue consistent with progression of CF disease. In addition, localized atelectasis was not uncommon and complete obstruction of airways was at times detected (FIGS. 23 and 24).

TABLE 7
Weight gain of CFTR^{-/-} kits reared on UDCA and omeprazole.

| Genotype | Birth Weight | Peak Weight | Weight at Death | Apparent |
|---|---|---|---|---|
| (Experiment) | In grams | (Age) | (Age) | Cause of Death |
| +/−(1) | 9.7 | 901 (180) | 901 (180) | Alive |
| −/−(1) | 11.3 | 1194 (165) | 1033 (180) | Alive |
| +/+(2) | 8.6 | 88.7 (19) | 89.2 (20) | — |
| −/−(2) | 8.9 | 31.9 (19) | 29.8 (20) | Acute Aspiration |
| | | | | (rare neutrophils) |
| +/+(3) | 8.6 | 168.2 (29) | 157.1 (32) | — |
| −/−(3) | 8.4 | 76.9 (27) | 71.7 (32) | Rectal Prolapse |
| +/+(4) | 9.1 | 41.7 (10) | 62.1 (13) | — |
| −/−(4) | 9.0 | 14.9 (10) | 14.2 (13) | Unknown |
| | | | | (focus of pneumonia noted) |
| +/−(5) | 10.4 | 28.9 (7) | 28.9 (7) | — |
| −/−(5) | 10.1 | 11.8 (6) | 10.2 (7) | Unknown |
| | | | | (intestinal obstruction noted) |

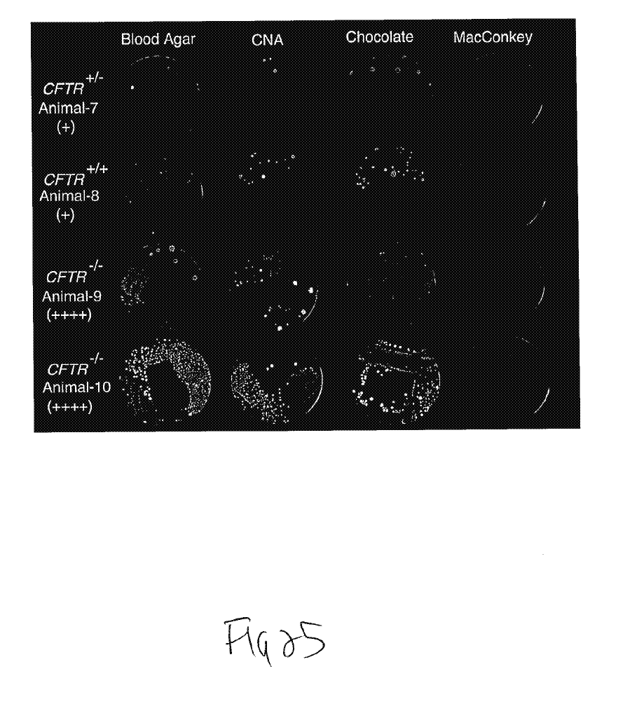

Bacteriology of bronchioalveolar lavages (BAL) fluid and fecal samples. Despite improved weight gain in treated CFTR^{-/-} kits that lack MI, approximately half still died within the first week of life. To evaluate the types of bacteria found in the lung in these and other CFTR^{-/-} kits that died within the first month of life, we performed bacteriologies on BAL fluid. To control for the type of bacteria deposited in the lung due to gut obstruction, CFTR^{-/-} kits that died from MI were evaluated (Table 8). As anticipated, those animals that succumb to MI had a much higher abundance of enteric bacterial flora in their lungs. By contrast, two additional species (Staphylococcus and Streptococcus) were found in the BAL of 2 day old CFTR^{-/-} kits without MI at higher abundance in comparison to wild type and heterozygous controls (Table 8 and FIG. 25). In CFTR^{-/-} kits that died after the first week of life, the density of bacteria in the lung was similar or only slightly greater than controls animals. In the oldest CFTR^{-/-} animal that died at 31 days, Streptococcus spp, alpha haemolytic was the only species found in the BAL and this differed from those found in the control. The types of bacteria found in feces from the oldest surviving CFTR^{-/-} and littermate control ferrets were also evaluated, however, no obvious differences were found (Table 8).

TABLE 8
Bacteriology of BAL and fecal samples

| Animal | CFTR | Age | | Density of | |
|---|---|---|---|---|---|
| (Experiment) | Genotype | (Days) | Sample | Growth ^{a} | Organism |
| 1 (1) | +/+ | 2 | BAL | + | Streptococcus spp, alpha haemolytic |
| 2 (1) | +/+ | 2 | BAL | − | No growth |
| 3 (1) | +/+ | 2 | BAL | + | Actinomyces spp |
| 4 (1) | −/− | 2 | BAL | ++++ | Staphylococcus spp; Enterococcus spp |
| 5 (1) | −/− (MI) ^{c} | 2 | BAL | ++ | Enterococcus spp |
| 6 (1) | −/− (MI) | 2 | BAL | ++++ | Bacillus spp |
| 7 (2) | +/− | 2 | BAL | + | Corynebacterium spp.; Enterococcus spp. |
| | | | | | Actinomyces spp |
| 8 (2) | +/+ | 2 | BAL | + | Streptococcus spp, alpha haemolytic; |
| | | | | | Enterococcus faecalis; Proteus mirabilis |
| 9 (2) | −/− (MI) | 2 | BAL | ++++ | Enterococcus faecalis; Coagulase |
| | | | | | negative Staph group; Corynebacterium |
| | | | | | spp; Chryseobacterium indologenes; |
| | | | | | Clostridium perfringens |
| 10 (2) | −/− | 2 | BAL | ++++ | Streptococcus spp, alpha haemolytic; |
| | | | | | Enterococcus species; Enterococcus faecalis; |
| | | | | | Staphylococcus epidermidis; |
| | | | | | Staph pseudintermedius; Proteus mirabilis |
| 11 (3) | +/− | 7 | BAL | + | Staph pseudintermedius |
| | | | | | Streptococcus spp, alpha haemolytic |
| 12 (3) | −/− | 7 | BAL | ++ | Streptococcus spp, alpha haemolytic |
| | | | | | Coag negative Staph group |
| | | | | | Clostridium perfringens |
| | | | | | Proteus mirabilis |
| 13 (4) | +/+ | 20 | BAL | ++ | Staph pseudintermedius; Streptococcus |
| | | | | | spp, alpha haemolytic |
| 14 (4) | −/− | 20 | BAL | ++ | Enterococcus faecalis; Staph pseudintermedius |
| 15 (5) | +/+ | 32 | BAL ^{b} | + | Coagulase negative Staph group; Non- |
| | | | | | fermenter species; Haemophilus spp |
| 16 (5) | −/− | 32 | BAL ^{b} | + | Streptococcus spp, alpha haemolytic |
| 17 (6-8) | +/− | 174 ¹ | Feces ^{d} | ++++ | Proteus vulgaris ¹; Proteus mirabilis ^{2, 3}; |
| | | 176 ² | | | Streptococcus spp, alpha haemolytic ^{2, 3}; |
| | | 1783 | | | E. coli ^{1, 2, 3}; Enterococcus faecalis ^{1, 2, 3}; |
| | | | | | Enterococcus faecium ³; Clostridium perfringens ^{1, 2}; |
| | | | | | Corynebacterium spp ³ |
| 18 (6-8) | −/− | 174 ¹ | Feces ^{d} | ++++ | Proteus mirabilis ^{1, 2, 3}; Streptococcus spp, |
| | | 176 ² | | | alpha haemolytic ^{1, 2, 3}; E. coli ^{1, 2, 3}; |
| | | 178 ³ | | | Enterococcus faecalis ^{1, 2} Enterococcus |
| | | | | | spp. ³; Enterococcus faecium ³; |
| | | | | | Clostridium perfringens ^{1, 2, 3} |

^{a} Evaluation of growth was based on bacterial colonies appearing in quadrant ¹ (+), ² (++), ³ (+++), ⁴ (++++) of the non-selective bacterial cultures plates.
^{b} Single lobe lavages was used to retain integrity of histopathology.
^{c} Animals that died from meconium ileus are denoted by (MI). All other CFTR^{-/-} animals passed stool.
^{d} Feces was collected every other day for three collections. The superscript number by the age at the time of collection was used to collate the presence of bacteria found on that day (as indicated in the last column).

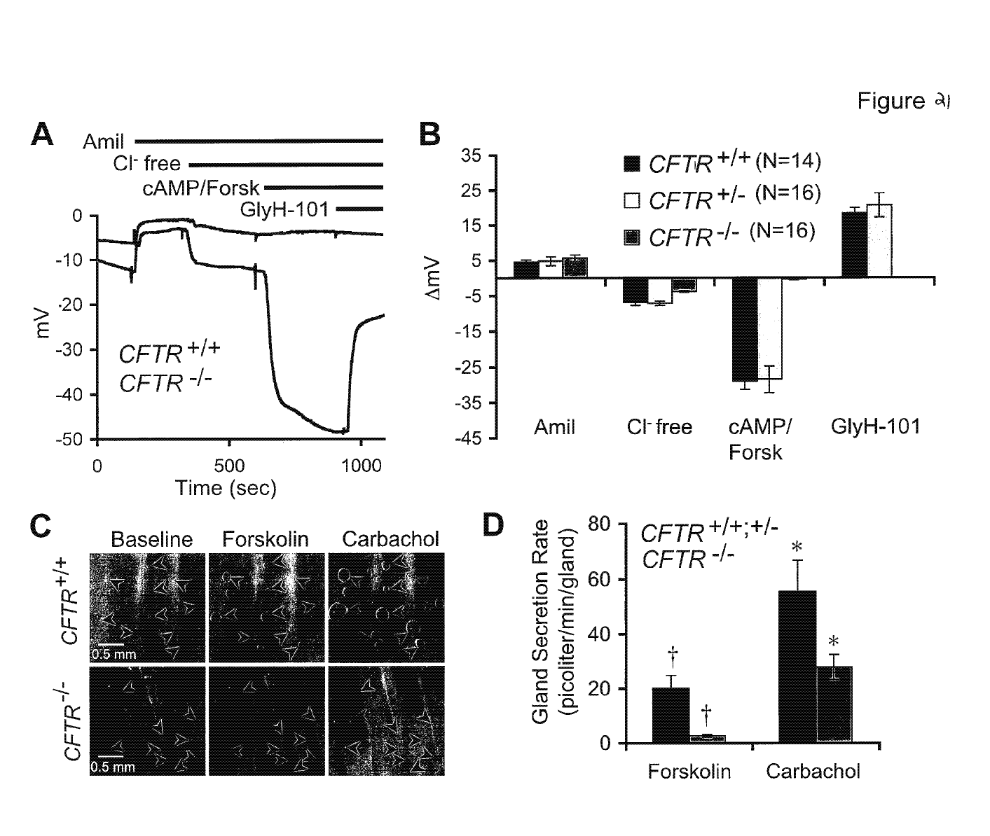

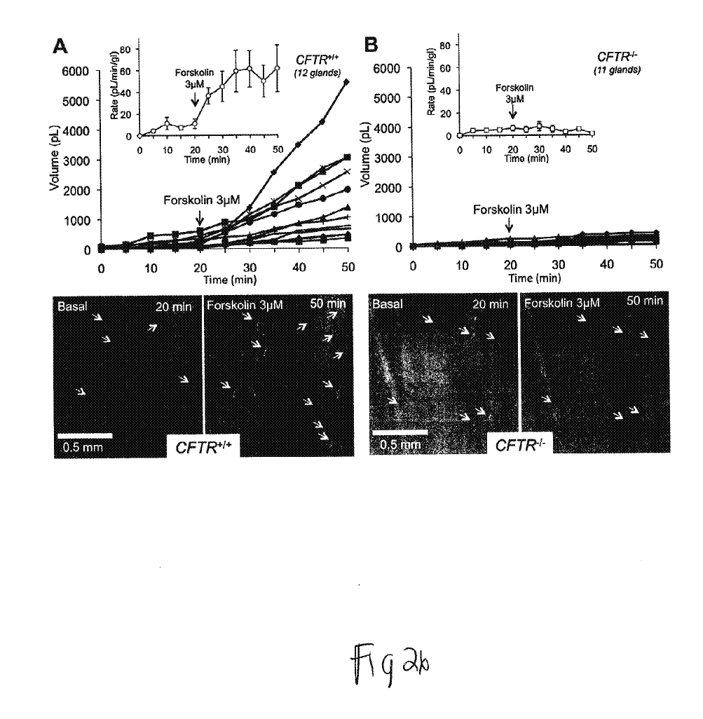

Tracheas from CFTR-knockout ferrets demonstrate defects in cAMP-induced chloride permeability and submucosal gland secretions. Lung disease is the most life-threatening aspect of CF (Welsh et al., 2001; Rowe et al., 2005). Defective CFTR-mediated anion transport and enhanced fluid absorption by the surface airway epithelium is thought to hinder bacterial clearance (Rowe et al., 2005). Additionally, submucosal glands of the cartilaginous airways express abundant CFTR, and have been proposed to play an important role in the pathogenesis of CF lung disease as a consequence of defective secretion of antibacterial factor-containing fluid into the airway lumen (Rowe et al., 2005; Wine et al., 2004; Salinas et al., 2005). Although the natural progression of lung disease in adult CFTR^{-/-} ferrets remains to be delineated, functional studies on newborn CFTR^{-/-} ferret tracheas demonstrate that this model features abnormalities characteristic of the proximal airways of CF patients, including defective cAMP-induced chloride permeability and submucosal-gland fluid secretion (FIG. 21). Using transepithelial potential difference (TEPD) measurements in an ex vivo tracheal xenograft model, we found that CFTR^{-/-} tracheas lack cAMP-dependent changes in Cl⁻ permeability (FIG. 21A-B), a feature that is characteristic of TEPD defects seen the nasal epithelium of human CF patients (Rowe et al., 2005). No significant difference was observed in amiloride-sensitive TEPD between the various genotypes; although this finding differs from studies of the human CF nasal epithelium that demonstrate elevated amiloride-sensitive changes in TEPD in comparison to non-CF (Rowe et al., 2005), it is consistent with findings from human CF and non-CF tracheal xenografts (Zhang et al., 1996) and thus may be a consequence of functional differences in the level of airway evaluated or a specific feature of the xenograft model. Fluid secretion from submucosal glands of CFTR^{-/-} tracheas was also significantly reduced-secretion in response to 3 μM forskolin was reduced 7.4-fold and secretion to 1 μM carbachol was reduced by half (FIGS. 21C-D and FIG. 26). Glandular secretory responses in human CF proximal airways show this same pattern—a large reduction in response to cAMP agonists and a smaller reduction in response to Ca²⁺ agonists (Wine et al., 2004; Salinas et al., 2005). There was no significant difference between CF and non-CF ferret tracheal xenografts in terms of the glandular area relative to the unit length of the surface airway epithelium (p=0.679), demonstrating that the observed differences in secretion were not due to altered gland size between genotypes.

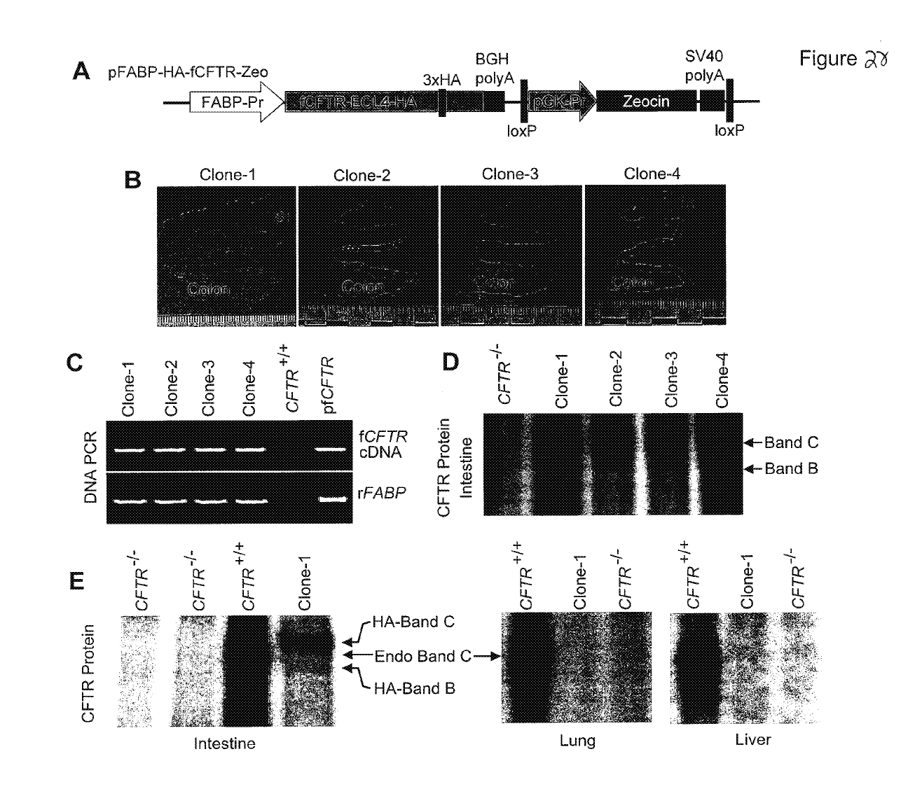

A gut-corrected transgenic CFTR-knockout ferret model corrects MI at birth. Similar to the situation for the CF pig and mouse models, the severity of intestinal complications in the CFTR^{-/-} ferret model significantly hinders its application as a research model. In CF mice, correction of intestinal complications by expressing the human CFTR cDNA under the direction of the intestinal fatty acid binding protein (FABPi) promoter has proven invaluable (Zhou et al., 1994). Taking advantage of this approach, a transgene cassette that expresses the fCFTR cDNA under the direction of the FABPi promoter was generated (FIG. 22A). This CFTR cDNA contains a HA-tag inserted into the 4^{th} extracellular loop, and demonstrated normal chloride channel function in vitro (data not shown). The linear fragment of the FABPi-HA-f CFTR-PGK-Zeo cassette was transfected into primary fibroblasts derived from a female CFTR^{-/-} 28-day embryo and selected pools of the transgenic cells were used for SCNT. Four transgenic FABPi-HA-f CFTR/CFTR^{-/-} clones were born harboring the transgene cassette in their genomic DNA (FIG. 22C), but only one survived the early post-natal period and passed stool normally (clone-1). The three additional clones born had to be euthanized within 36 hours after birth due to MI (FIG. 22B, clone-2, -3, and -4). The surviving clone-1 was also euthanized to determine the expression patterns of recombinant fCFTR and this clone had a grossly normal intestine lacking any signs of MI (FIG. 22B, clone-1). Analysis of intestinal fCFTR expression in the four clones demonstrated that clone-1 expressed the highest levels of CFTR protein (FIG. 22D), correlating with the lack of a neonatal MI phenotype in this clone. Additionally, primary fibroblasts, liver and lung were harvested from clone-1 to confirm tissue-specific expression of CFTR prior to nuclear transfer recloning and expansion of the line. Results from this analysis demonstrated that FABPi-HA-fCFTR/CFTR^{-/-} clone-1 expressed fCFTR in the intestine but not in the lung and liver (FIG. 22E). Endogenous fCFTR protein was expressed in all of these organs from CFTR^{+/+}, but not CFTR^{-/-}, kits (FIG. 22E). Currently, SCNT is being performed to expand the clone-1 line.

### Discussion

Although the gene responsible for cystic fibrosis was discovered more than two decades ago, it still remains unclear how CFTR defects lead to the airway disease that is responsible for most CF deaths. Such slow progress can be attributed to the lack of an adequate animal model of CF airway disease. Although CF mice have illuminated so many aspects of CF pathophysiology, they do not display a human-like CF airway disease. The need for different animal models of CF prompted the creation of CF pig and ferret models. This first report of early phenotypic features in a genetically engineered CF ferret model demonstrates that it shares many of the abnormalities seen in newborn CF humans. CF is likely the first human disease for which directed engineering has generated two non-rodent knockout models and comparative studies on organ-specific CF disease phenotypes in four species (human, mouse, ferret, and pig) will greatly expand our understanding of CF pathogenesis.

The greatest cause of CF neonatal mortality in all species is intestinal obstruction, but with important differences in penetrance, age at onset, and phenotype. CF humans, pigs and ferrets display a true meconium ileus at birth, whereas CF mice develop intestinal complications at a different stage in development (i.e., weaning to solid chow). The penetrance of meconium ileus varies greatly between CF humans (about 15%), ferrets (about 75%) and pigs (100%), and a wide variance in intestinal obstruction is also observed in CF mice (0-100%) depending on the strain background (Egan, 2009; Guilbault et al., 2007). Modifier genes appear to influence the occurrence of meconium ileus in CF infants based on monozygous and dizygous CF twin/triplet studies (Blackman et al., 2006), and our studies also suggest that the hob exerts a genetic influence for development of meconium ileus in CFTR^{-/-} ferrets. Complete penetrance of meconium ileus in the CFTR^{-/-} piglets studied to date may be a consequence of their intestinal anatomy and/or their inbred status.

The severity of CF pancreatic pathology at birth differs widely across the species, with pigs being most severe (Rogers et al., 2008), ferrets and humans being similar (Oppenheimer et al., 1973; Oppenheimer et al., 1975), and mice least severe (Guibault et al., 2007). The sparing of the pancreas in newborn mice has been attributed to alternative Catactivated chloride channels in this organ (Gray et al., 1994), and it will be interesting to determine if this single factor can account for the variation across all four species. The vas deferens is another tissue that demonstrates variable CF disease pathology between the species. Congenital bilateral absence of the vas deferens is diagnosed in nearly all (about 99%) of adult males with CF (Jarzabek et al., 2004). Even though “congenital absence” implies that this structure is not present at birth, most CF infant males have an intact vas deferens at birth (Oppenheimer et al., 1975) and increased detection rate in younger patients suggests that disease in this tissue is a degenerative process that may begin prior to birth in some individuals and culminate with destruction by adulthood (Gaillard et al., 1967). Interestingly, with the exception of the CF ferret that demonstrates an absent or degenerate vas deferens at birth, other CF animal models have not replicated this phenomenon CF mice have minor histologic changes to the vas deferens, but are fertile (Reyhaert et al., 2000), and CF pigs appear to have intact vas deferens at birth (Rogers et al., 2008). Whether CF pigs will develop disease of the vas deferens in adulthood remains to be determined.

Of all species, CF newborn ferrets have the most severely impaired nutritional status at birth. The ferret intestinal tract is unique in comparison to human, pig and mice. As an obligate carnivore, ferrets lack a cecum, which is found in humans, pigs, and mice; this structure is known to assist in the digestion of plant material. The ferret also has a shorter intestinal transit time (75+/−54 minutes) in comparison to other species (Schwarz et al., 2003; Bell; 1999). These features likely impose special nutrient requirements, which to date remain to be defined at the molecular level. The complete lack of weight gain in newborn CF ferrets suggests that intestinal CFTR plays a critical role in nutritional absorption in this species. Additionally, the pancreatic pathology and bile acid-dependent liver abnormalities observed at birth in CF ferrets may also play a role in their poor nutrition.

Oral administration of a proton pump inhibitor significantly improved nutrition in CF ferrets, suggesting that CFTR-dependent perturbations in gastrointestinal (GI) pH may have a significant impact on the absorption of fat and/or other key nutrients in this species. Proton pump inhibitors have been extensively used in CF patients suffering sustained steatorrhoea while on pancreatic enzymes, and these drugs appear to improve nutritional status according to some reports (Proesmans et al., 2003; Tran et al., 1998). Furthermore, reduced GI pH in a CFTR^{-/-} mouse model has been suggested to impair lipolysis and fat absorption by the intestine (WT: 94+/−0.3% vs KO: 89.7+/−1.2%), despite normal lipase and bicarbonate secretion by the pancreas (Bijvelds et al., 2005), and this defect was corrected by oral administration of a proton pump inhibitor. Interestingly, elevated bile salt secretion into the feces was also observed in CFTR^{-/-} mice, despite normal levels of bile salt secretion by the biliary system (Bijvelds et al., 2005). Based on the present study, it is concluded that CFTR^{-/-} mice suffer from fat malabsorption due to impairment of the duodenal bicarbonate production that is required for efficient lipolysis and uptake of fatty acids (Bijvelds et al., 2005). It is interesting, however, that 4F508-CFTR mice do not suffer from the same lipolysis and fatty acid uptake defects seen in CFTR^{-/-} mice (Bijvelds et al., 2005). Although further studies on intestinal biology in the ferret are needed to understand how CFTR in this organ might directly influence lipid absorption in this species, it seems plausible that CFTR^{-/-} ferrets suffer from a relatively pronounced intestinal pH imbalance that influences fat absorption in a similar manner as in CFTR^{-/-} mice.

Understanding how CFTR malfunction contributes to the extreme GI phenotypes in these CF animal models should help to improve management of the more subtle pathologies seen in CF humans. However, a major impetus to controlling or genetically eliminating the GI pathology is to allow the models to grow to maturity so that the natural progression of lung disease can be determined. Thus far, the basic defects in airway bioelectric and submucosal gland secretory properties reproduce that seen in human CF. Furthermore, rearing CFTR^{-/-} kits under protocols that improve nutrition did not completely prevent early fatal lung infection that were also seen in nutritional compromised animals. Despite improved nutrition, neonatal CFTR^{-/-} kits had increased bacterial counts and isolates from BAL fluid that subsided after the first week. After the first week, lung infections did not appear to be the primary cause of death in CFTR^{-/-} kits. It is interesting to speculate that neonatal aspiration events may serve as an inoculation mechanism and defective bacterial eradication compromises the ability of the CF lung to clear normal early pathogens like control animals. Although the number of animals analyzed remains low, the observation of early fatal lung infections and rectal prolapse in CFTR^{-/-} kits is also similar to clinical observations in CF infants during the early 1950's (Bodian, 1953). In this case study of 68 CF infants, over 60% died before the age of 1 year, while only 15% survived 2-13 years. Greater than half of the CF infants who contracted lung infection at birth died (excluding those with MI) and failure to thrive during the neonatal period despite a ravenous appetite was common. These observations are quite similar to the findings herein with CFTR^{-/-} kits. As with humans, the improved use of broad-spectrum antibiotics during the neonatal period may help to improve survival in this CF ferret model. These initial findings in the lung and other organs of CFTR^{-/-} ferrets, suggest that the ferret may be a useful model for dissecting CF pathophysiology and developing therapies. These studies also describe the production of the first transgenic ferret using SCNT and such methods will expand opportunities generally for genetically dissecting pathogenesis in the ferret model.

### REFERENCES

Altschu et al., J. Mol. Biol., 215:403 (1990).
Bell, Vet. Clin. North Am. Exot. Anim. Pract., 2:169 (1999).
Berthelot et al., Livestock Production Science, 83:73 (2003).
Bijvelds et al., Am. J. Physiol. Gastrointest. Liver Physiol., 288:G646.
Blackman et al. Gastroenterology, 131:1030 (2006).
Bodian, 1953. Fibrocystic disease of the pancreas: a congenital disorder of mucus production-mucosis, New York: Grune & Stratton, ix, 244 p. pp. (1953).
Bolton-Maggs and Pasi, Lancet, 24:361(9371):1801-9 (2003).
Cho et al., Anim. Reprod. Sci., 2002; 73:151 (2002).
Choi et al., J. Anat., 197 Pt 3:361 (2000).
Choi et al., J. Anat., 3:361 (2000).
Choi et al., J. Clin. Invest., 117:3118 (2007).
Concannon and Gatti, Hum. Mutat., 10:100 (1997).
Cotting et al., Gut, 31:918 (1990).
Dai et al., Nat. Biotechnol., 20:251 (2002).
Dajani et al., Am. J. Respir. Cell Mol. Biol., 32:548 (2005).
Deconinck and Dan, Pediatr. Neurol., 1:1 (2007).
Dobrinsky et al., Theriogenology, 57:285 (2002).
Dobrinsky, Theriogenology, 45:17 (1996).
Dobrinsky, Theriogenology, 56:1333 (2001).
Drumm et al., N. Engl. J. Med., 353:1443 (2005).
Egan, Drug Discovery Today: Disease Models, 6:35 (2009).
Engelhardt et al., Nat. Genet., 2:240 (1992).
Fox et al., J. G. Fox, editor. Baltimore: Williams & Wilkins. 211 (1998).
Gaillard et al., J. Urol., 158:1549 (1997).
Gattone V., Current Opinion in Pharmacology, 5:535 (2005).
Glozman et al., J. Cell Biol., 184:847 (2009).
Gray et al., Am. J. Physiol., 266:C213 (1994).
Grosse-Hovest et al., Proc. Natl. Acad. Sci. U.S.A., 101:6863 (2004).
Grubb et al., Am J Physiol., 267:C293 (1994).
Gudmundsdottir and Ashworth, Oncogene, 25:5864 (2006).
Heeckeren et al., J. Clin. Invest., 100:2810 (1997).
Guilbault et al., Am. J. Respir. Cell Mol. Biol., 36:1 (2007).
Harries et al., Arch. Dis. Child., 54:19 (1979).
Hendrie and Russell, Molecular Therapy, 12:9 (2005).
Hofmann et al., J. Lipid. Res., 33:617 (1992).
Hofmann, Hepatology, 49:1403 (2009).
Jarzabek et al., Reprod. Biol., 4:119 (2004).
Joo et al., Am. J. Physiol. Lung Cell Mol. Physiol., 281:L458 (2001).
Kizilova et al., Ontogenez, 29:429 (1998).
Kong et al., Theriogenology, 2000; 53:1817 (2000).
Kuroiwa et al., Nat. Genet., 36:775 (2004).
Lai et al., Science, 295:1092 (2002).
Leibo et al., Theriogenology, 57:303 (2002).
Leigh et al., 1986 Exp. Lung Res., 10:153 (1986).
Levine-A J, Cell, 88:323 (1997). Lohmann, Hum. Mutat., 14:283 (1999).
Li et al., Dev. Biol., 293:439 (2006).
Li et al., J. Exp. Zoolog. A Comp. Exp. Biol., 303:1126 (2005).
Li et al., Reprod. Biol. Endocrinol., 1:83 (2003).
Li et al., Theriogenology, 66:183 (2006).
Lindblad et al., Hepatology, 30:1151 (1999).
Lindeberg et al., Theriogenology, 57:2167 (2002).
Lindeberg et al., Theriogenology, 60:1515 (2003).
Liu et al., Am. J. Respir. Cell Mol. Biol., 34:56 (2006).
Liu et al., Am. J. Respir. Cell. Mol. Biol., 36:313 (2007).
Liu et al., Gene. Ther., 14:1543 (2007).
Lusis et al., Annu. Rev. Genomics Hum. Genet., 5:189 (2004).
Martina et al., Nature, 425:915 (2003).
McCreath et al., Nature, 405:1066 (2000).
Mercer et al., Am. J. Respir. Cell Mol. Biol., 10:613 (1994).
Ollett, J. Path. Bact., 59:357 (1947).
Oppenheimer et al., Arch. Pathol., 96:149 (1973).
Oppenheimer et al., Perspect. Pediatr. Pathol., 2:241 (1975).
Ostedgaard et al., Proc. Natl. Acad. Sci. U.S.A., 104:15370 (2007).
Pack et al., Cell Tissue Res., 208:65 (1980).
Piltti et al., Theriogenology, 61:811 (2004).
Plopper et al., Exp. Lung Res., 1:171 (1980).
Proesmans et al., Eur. J. Pediatr., 162:760 (2003).
Quinn, Clinical veterinary microbiology, [London]: Wolfe. 648 p. pp. (1994).
Reynaert et al., Mol. Reprod. Dev., 55:125 (2000).
Riordan et al., Science, 245:1066 (1989).
Robinson et al., J. Anat., 145:173 (1986).
Rogers et al., Science, 321:1837 (2008).
Rowe et al., N. Engl. J. Med., 352:1992 (2005).
Russell et al., Nat. Genet., 18:325 (1998).
Salinas et al., FASEB J., 19:431 (2005).
Scher et al., Ann. Pharmacother., 31:1003 (1997).
Schnieke et al., Science, 278:2130 (1997).
Schwarz et al., Vet. Radiol. Ultrasound, 44:165-172.
Scott-Jupp et al., Arch. Dis. Child., 66:698 (1991).
Sehgal et al., Am. J. Respir. Cell Mol. Biol., 15:122 (1996).
Steinberg M H, Trends Pharmacol. Sci., 27:204 (2006).
Subbarao et al., PLoS Pathog., 3:e40 (2007).
Sun et al., J. Clin. Invest., 118:1578 (2008).
Tatiana et al., FEMS Microbiol. Lett., 174:247 (1999).
Tesson et al., Transgenic Res., 14:531 (2005).
Tharasanit et al., Reproduction, 129:789 (2005).
Tran et al., Acta Paediatr., 87:553 (1998).
Vajta et al., Acta. Vet. Scand., 1997; 38:349 (1997).
Verkman et al., Am. J. Physiol. Cell Physiol., 284:C2 (2003).
Walker et al., Cloning Stem Cells, 4:105 (2002).
Walker, Lancet, 369:218 (2007).
Wang et al., Am. J. Respir. Cell Mol. Biol., 24:195 (2001).
Weber et al., Acta Paediatr. Scand. Suppl., 317:9 (1985).
Weber et al., N. Engl. J. Med., 289:1001 (1973).
Wells et al., Theriogenology, 59:45 (2003).
Welsh et al., The Metabolic and Molecular Basis of Inherited Disease. New York: McGraw-Hill (2001).
Whittingham et al., Science, 178:411 (1972).
Wilmut et al., Nature, 385:810 (1997).
Wine et al., Proc. Am. Thorac. Soc., 1:47 (2004).
Ya et al., Mustela erminea. Scientifur., 17(2):127 (1993).
Yan et al., J. Biol. Chem., 281:29684 (2006).
Yang et al., Nat. Genet., 39:295 (2007).
Zhang et al., Am. J. Physiol., 270:C1326 (1996).
Zhou et al., Science, 266:1705 (1994).

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A transgenic Mustelidae model of a human disease or condition, in which a gene associated with the disease or condition comprises a targeted genetic modification, wherein the transgenic Mustelidae optionally comprises a transgene encoding a gene product of interest.

2. The transgenic Mustelidae of claim 1 which is a ferret.

3. The transgenic Mustelidae of claim 1 which is an ermine, badger, otter, skunk or wolverine.

4. The transgenic Mustelidae of claim 1 wherein the disease or condition is cystic fibrosis.

5. The transgenic Mustelidae of claim 1 wherein the gene comprising the modification is a cystic fibrosis transmembrane conductance regulator gene.

6. The transgenic Mustelidae of claim 1 which comprises the transgene.

7. The transgenic Mustelidae of claim 6 wherein the transgene comprises a tissue-specific promoter operably linked to an open reading frame encoding the gene product of interest.

8. The transgenic Mustelidae of claim 6 wherein the gene product is a heterologous gene product.

9. The transgenic Mustelidae of claim 7 or 8 wherein the gene product is a cystic fibrosis transmembrane conductance regulator.

10. The transgenic Mustelidae of claim 1 which is heterozygous for the modification.

11. The transgenic Mustelidae of claim 1 which is homozygous for the modification.

12. The transgenic Mustelidae of claim 1 wherein the modification inhibits or prevents expression of the gene.

13. The transgenic Mustelidae of claim 1 wherein the modification inhibits or prevents expression of the gene product encoded by a corresponding gene that does not include the targeted modification.

14. The transgenic Mustelidae of claim 1 wherein the modification comprises an insertion of a heterologous nucleic acid molecule.

15. The transgenic Mustelidae of claim 14 wherein the heterologous nucleic acid molecule encodes a gene product and the insertion is targeted 3′ to a promoter in the endogenous sequences.

16. The transgenic Mustelidae of claim 1 wherein the modification is a deletion.

17. The transgenic Mustelidae of claim 1 wherein the modification is a replacement of endogenous sequences.

18. An isolated transgenic cell of the transgenic Mustelidae of claim 1.

19. The cell of claim 18 wherein the cell is a fetal cell.

20. The cell of claim 18 wherein the cell is an adult cell.

21. The cell of claim 18 wherein the cell is a fibroblast.

22. A method of making a transgenic Mustelidae model of a disease or a condition, comprising:
a) providing a Mustelidae donor cell comprising an expression vector comprising a gene associated with a disease or condition or comprising a targeted disruption vector in an endogenous gene, which disruption results in a disease or condition;
b) introducing the nucleus of the donor cell or the donor cell into an enucleated Mustelidae oocyte to generate an embryo; and
c) transferring the embryo into a surrogate female.

23. A method to cryopreserve Mustelidae embryos, comprising:
a) providing one or more Mustelidae morula embryos, Mustelidae compact morula embyros or Mustelidae early blastocyst embryos in cryopreservation medium;
b) introducing one or more of the embryos to a vessel with an inner diameter of about 0.2 mm to about 0.3 mm and a wall thickness of about 0.01 mm to about 0.05 mm; and
c) subjecting the vessel with the one or more embryos to vitrification.

24. The method of claim 23 wherein the embryo is from a nontransgenic Mustelidae.

25. The method of claim 23 wherein the embryo is from a transgenic Mustelidae.

26. The method of claim 23 in which cytoplasmic lipids are not removed from the one or more embryos by centrifugation.

27. A method to enhance the number of live offspring from cryopreserved Mustelidae embryos, comprising:
a) thawing one or more of the embryos of claim 23; and
b) transferring thawed embryos to pseudopregnant females within less than 48 hours.

28. The method of claim 27 wherein the cryopreservation medium comprises an effective amount of one or more cryoprotectants.

29. The method of claim 27 wherein the one or more cryoprotectants include dimethylsulfoxide, ethylene glycol, glycerol, propylene glycol, sucrose or trehalose.

30. A method to cryopreserve transgenic Mustelidae cells, comprising:
a) introducing one or more Mustelidae donor cells to a Mustelidae zona pellicidae that substantially lacks cytoplasm to form a pseudoembryo;
b) introducing one or more of the pseudoembryos to a vessel with an inner diameter of about 0.2 mm to about 0.3 mm and a wall thickness of about 0.01 mm to about 0.05 mm; and
c) subjecting the vessel with the one or more pseudoembryos to vitrification.

31. The method of claim 30 wherein the donor cells are transgenic Mustelidae donor cells comprising an expression vector comprising an open reading frame encoding a gene product or comprising a targeted disruption vector.

32. A method of propagating a selected phenotype in a Mustelidae, comprising:
a) providing a Mustelidae donor cell from a Mustelidae with a selected phenotype;
b) introducing the nucleus of the donor cell or the donor cell into an enucleated Mustelidae oocyte to generate an embryo;
c) transferring the embryo into a surrogate female; and
d) identifying progeny derived from the transferred embryo with the selected phenotype.

33. The method of claim 32 further comprising breeding progeny with the selected phenotype to obtain further progeny with the selected phenotype.

34. The method of claim 22 or 32 wherein the Mustelidae donor cell is a fibroblast cell.
